(19) United States
(12) Patent Application Publication
(10) Pub. No.: US 2023/0295642 A1
(43) Pub. Date: Sep. 21, 2023

(54) COMPOSITIONS AND METHODS FOR ENZYME CATALYZED TOEHOLD MEDIATED STRAND DISPLACEMENT (TMSD)

(71) Applicant: RUTGERS, THE STATE UNIVERSITY OF NEW JERSEY, New Brunswick, NJ (US)

(72) Inventors: Smita S. Patel, Piscataway, NJ (US); Laura C. Johnson, Piscataway, NJ (US); Gayatri Patel, Basking Ridge, NJ (US); Anupam Singh, Piscataway, NJ (US)

(73) Assignee: RUTGERS, THE STATE UNIVERSITY OF NEW JERSEY, New Brunswick, NJ (US)

(21) Appl. No.: 18/160,774

(22) Filed: Jan. 27, 2023

Related U.S. Application Data
(60) Provisional application No. 63/303,880, filed on Jan. 27, 2022.

(51) Int. Cl. C12N 15/67

(52) U.S. Cl. CPC C12N 15/67

(57) ABSTRACT

Compositions and methods for rapid and efficient Toehold mediated strand displacement (TMSD) reactions are disclosed.

### CROSS REFERENCE TO RELATED APPLICATIONS

This patent application claims the benefit of U.S. Provisional Patent Application No. 63/303,880, filed Jan. 27, 2022. The entire contents of the foregoing application are incorporated herein by reference, including all text, tables, drawings, and sequences.

### STATEMENT REGARDING GOVERNMENT FUNDING

This invention was made with government support under grant number GM118086 awarded by The National Institutes of Health. The government has certain rights in the invention.

### INCORPORATION-BY-REFERENCE OF MATERIAL SUBMITTED IN ELECTRONIC FORM

The Contents of the electronic sequence listing (RUT-108-US.xml; Size: 51,215 bytes; and Date of Creation: Jan. 27, 2023) is herein incorporated by reference in its entirety.

### FIELD

The present invention relates to the fields of molecular engineering and improved helicase variants which increase the rate of TMSD.

### BACKGROUND

Several publications and patent documents are cited throughout the specification in order to describe the state of the art to which this invention pertains. Each of these citations is incorporated by reference herein as though set forth in full.

Toehold mediated strand displacement (TMSD) typically starts with a double-stranded DNA complex composed of the original strand and the protector strand. The original strand has an overhang region, known as a “toehold” which is complementary to a third strand of DNA referred to as the “invading strand”. The invading strand is a sequence of single-stranded DNA (ssDNA) which is complementary to the original strand. The toehold regions initiate the process of TMSD by allowing the complementary invading strand to hybridize with the original strand, creating a DNA complex composed of three strands of DNA. This initial endothermic step is rate limiting and can be tuned by varying the strength (length and sequence composition e.g. G-C or A-T rich strands) of the toehold region. The ability to tune the rate of strand displacement over a range of 6 orders of magnitude generates the backbone of this technique and allows the kinetic control of DNA or RNA devices.

After the binding of the invading strand and the original strand occurred, branch migration of the invading domain then allows the displacement of the initial hybridized strand (protector strand). The protector strand can possess its own unique toehold and can, therefore, turn into an invading strand itself, starting a strand-displacement cascade. The whole process is energetically favored and although a reverse reaction can occur its rate, is up to 6 orders of magnitude slower. Additional control over the system of toehold mediated strand displacement can be introduced by toehold sequestering.

### SUMMARY

In accordance with the present invention, a soluble, stable, isolated or purified truncated twinkle enzyme comprising a deletion of amino terminal domain of the mature form of the twinkle enzyme is provided, wherein said truncated twinkle enzyme exhibits increased solubility and catalyzes toehold mediated strand displacement reactions. In certain embodiments, the isolated or purified truncated twinkle enzyme comprises the carboxy terminal domain (CTD) of SEQ ID NO: 18. In other embodiments, the CTD twinkle enzyme comprises a sequence tag which enhances solubility of the truncated protein. The tag may optionally be SUMO and comprises SEQ ID NO: 19 or SEQ ID NO: 20. In certain approaches, the CTD twinkle enzyme is affixed to a nanoparticle.

Also disclosed are nucleic acids encoding each of the CTD twinkle truncation variants described above. Host cells comprising vectors encoding such nucleic acids also form an aspect of the invention.

In yet another embodiment, a method for rapid and efficient toe hold mediated strand displacement (TMSD) is disclosed. An exemplary embodiment entails contacting a double-stranded DNA complex comprising a target strand and an incumbent strand, said target strand comprising overhanging toe-hold sequence which is complementary to a third invading DNA strand, said invading strand being single stranded and complementary to the target strand; with an effective amount of a C terminal variant twinkle enzyme of SEQ ID NO:19 or 20; initiating TMSD under hybridizing conditions such that the complementary invading strand hybridizes with the target strand, creating a DNA complex composed of three strands of DNA, wherein branch migration of the invading strand causes displacement of the incumbent strand. The method can be used to advantage in a variety of assays, including without limitation, detection of single nucleotide polymorphisms and genetic copy number variations, detection of biomarkers indicative of increased cancer risk and assays for genotyping viral or bacterial strains.

Also provided is a kit for practicing any of the foregoing methods. An exemplary kit can comprise a purified, stable, soluble CTD operably linked to a SUMO tag of SEQ ID NO: 19 and a buffer suitable for TMSD reactions for example. The kit may also further comprise positive and negative control sequence constructs for assessing accuracy of TMSD reactions,

### BRIEF DESCRIPTION OF THE DRAWINGS

FIGS. 1A-1L. Twinkle catalyzes TMSD reactions. FIG. 1A. Schematic depicting basic steps of toehold mediated DNA strand displacement (TMSD) reaction. Target substrate DNA is composed of annealed incumbent strand (IncS) and target strand (TS). The four dG residues at the 5′end of TS quench the fluorescence signal from fluorescein (FAM) moiety linked to the 3′ end of the IncS. The reaction initiates with the docking of invader strand (IS) on the toehold. The IncS is displaced by IS following the branch migration step. Increase in the fluorescence signal due to complete displacement of IncS with time is used to measure the rate of TMSD reaction. FIG. 1B. Experimental setup used for the stopped-flow experiments. FIG. 1C. Real-time TMSD traces from stopped-flow assay. The rate of TMSD reaction in presence of Twinkle was 1.2 min⁻¹. No change in fluorescence signal was observed in absence of Twinkle or in absence of Twinkle and IS. FIG. 1D. Native PAGE showing kinetics of TMSD reactions performed in presence and in absence of Twinkle. FIG. 1E. For each time-point, fraction of IncS displaced was quantified, plotted against time and fitted to exponential equation. FIG. 1F. Bar chart showing the initial rate of IncS displacement (in fraction·min⁻¹) determined for the reactions conducted with or without Twinkle. FIG. 1G. Scheme of stopped-flow experiments to study the effect of Twinkle concentration or IS concentration on rate of Twinkle catalyzed TMSD. FIGS. 1H and 1I. Rates of TMSD reactions conducted at different concentrations of Twinkle (FIG. 1H) and at different concentrations of IS (FIG. 1I). FIG. 1J-1L. Schematic of TMSD reaction with shorter IncS and TS (FIG. 1J). Representative traces from stopped-flow assay to measure real-time rates of TMSD reactions performed with and without Twinkle (FIG. 1K). Rates of TMSD reactions carried out in presence and in absence of Twinkle (FIG. 1L).

FIGS. 2A-2D. Twinkle catalyzed strand-displacement reactions require toehold formation. FIG. 2A. Schematic depicting strand-displacement reactions in presence of IS with ‘toe’ (TMSD, left panel) and in presence of IS without ‘toe’ (recombination, right panel). FIG. 2B. Real-time traces of strand-displacement reactions with different ISs. FIG. 2C. Native PAGE showing strand displacement reactions with IS with toe (left) and with IS lacking the toe (Right). FIG. 2D. Fraction of IncS displaced plotted as a function of time and fitted to exponential equation.

FIGS. 3A-3E. Controlling the rate of Twinkle catalyzed TMSD. FIG. 3A. Schematic representation of different substrate designs used for conducting Twinkle catalyzed TMSD reactions. FIG. 3B. Fractions of IncS displacement obtained from Native PAGE experiments with different substrates. FIG. 3C. Initial rates of TMSD reactions conducted with stated substrates. FIG. 3D. Scheme of a TMSD reaction performed on target DNA substrate with a TS without a 3′ overhang beyond the toehold. FIG. 3E. Fluorescence traces from real-time stopped-flow assay to measure the rates of TMSD reactions with the substrates shown in FIG. 3D.

FIGS. 4A-4E. Twinkle catalyzed TMSD does not require Twinkle's translocation on the DNA substrates. FIG. 4A. Representative kinetics of Twinkle catalyzed TMSD reactions performed with Mg⁺² and ATP. FIG. 4B. Representative kinetic traces of Twinkle catalyzed TMSD reactions performed with IS lacking the toe. FIGS. 4C and 4D. Fractions of IncS displaced obtained from gel-based assays plotted as a function of time and fitted to exponential equation. Twinkle catalyzed strand-displacement reactions with IS with and without a toe FIG. 4C and FIG. 4D, respectively). FIG. 4E. Bar chart showing initial rates of Twinkle catalyzed IncS displacement.

FIGS. 5A-5E. Twinkle catalyzed TMSD is independent of the direction of branch migration. FIG. 5A. Schematic representation of the TMSD substrates with direction of IS branch migration in 5′ to 3′ direction (5′ toehold, Left panel) and 3′ to 5′ direction (3′ toehold, Right panel). FIG. 5B. Experimental conditions for the stopped-flow assay used to measure rates of TMSD reactions. FIG. 5C. Representative fluorescence traces showing real-time displacement of IncS for the TMSD reactions conducted with substrates shown in FIG. 5A. FIG. 5D. Fractions of IncS displaced from gel-based assay plotted as a function of time and fitted to exponential trend. FIG. 5E. Rate of IncS displacement (in fraction·min⁻¹) as obtained from the fitting of the data in FIG. 5D.

FIGS. 6A-6C. Twinkle catalyzed TMSD reactions are sensitive to nucleotide mismatches. FIG. 6A. Substrate design to study effect of nucleotide mismatches on Twinkle catalyzed TMSD. FIG. 6B. Fraction of IncS displaced for each time point obtained from gel-based assays, plotted against time and fitted to exponential equation. FIG. 6C. Bar chart showing the initial rates of InCS displacement (in fraction·min⁻¹) for the reactions conducted with stated substrates.

FIGS. 7A-7C. FIG. 7A. Diagram of full-length Twinkle domain structure. FIG. 7B. Schematic of Twinkle constructs model of the Twinkle monomer. Full-length Twinkle (FL TWN) contains an N-terminal domain (NTD), linker (pink), and C-terminal domain (CTD). Various truncations of NTD are shown along with the completely N-truncated Twinkle CTD (TWN CTD). FIG. 7C. Model of Twinkle shows the NTD, linker, CTD and the residue 360 (green sphere).

FIGS. 8A-8D. Twinkle CTD catalyzes TMSD. FIG. 8A. Schematic showing the substrate design used for the experiment. FIG. 8B. Native PAGE show kinetics of TMSD reactions performed with full-length Twinkle (Left) and Twinkle CTD (Right). FIG. 8C. Fractions of IncS displaced obtained from the gels shown in FIG. 8D. Rates of Twinkle catalyzed displacement of IncS.

FIGS. 9A-9G: Twinkle catalyzes TMSD reactions. (FIG. 9A) Schematic depicting basic steps of TMSD reaction and fluorescence-based strategy to measure reaction rates. Target substrate dsDNA is composed of annealed protector strand (PS, γ′₁₅) and target strand (TS, β₇γ₁₅). The four dG residues at the 3′-end of TS quench the fluorescence from fluorescein (FAM) moiety linked to the 5′-end of the PS. The reaction initiates with the docking of the invader strand (IS, β′₇γ′₁₅) on the toehold. PS is displaced by IS following the branch migration step. Increase in the fluorescence due to complete displacement of PS is used to measure the rate of TMSD reaction in real-time on a stopped flow device. The Greek alphabets with and without apostrophe sign represent complementary DNA domains while the numerical values in the subscripts depict the number of nucleotides constituting these domains. The toehold and branch migration domains present in TS, IS and PS in black and red colors, respectively. (FIG. 9B) Reaction conditions used to conduct the stopped-flow experiments for the determination of rates of spontaneous and catalyzed TMSD reactions. (FIG. 9C) Representative fluorescence traces showing uncatalyzed TMSD reactions in absence of an enzyme (blue) or in presence of bacteriophage T7 helicase gp4A′ (red) and a catalyzed TMSD reaction in presence of Twinkle (orange). No increase in fluorescence was observed in absence of IS and in absence or presence of Twinkle (green and black, respectively). (FIG. 9D) Bar chart showing the observed rates of TMSD (k_{obs}, in s⁻¹) determined for the reactions conducted with T7gp4A′ or Twinkle, or in absence of any enzyme (−Twinkle). Rates were determined by fitting the stopped flow traces to single exponential equation. (FIG. 9E) Bar charts showing the observed rates of TMSD (k_{obs}) reactions with 10 nM target dsDNA, 40 nM or 100 nM IS and different concentrations of Twinkle hexamer (0 nM to 80 nM). Bars show the mean k_{obs} from multiple repeats (shown as circles). Error bars represent standard deviation of the means. (FIG. 9F) k_{obs} from FIG. 9E plotted as function of Twinkle concentration and fitted to hyperbola. (FIG. 9G) Twinkle K_{M} determined from the hyperbolic fitting of data in FIG. 9F. Error bars are the standard from the fitting.

FIG. 10A-10G: Twinkle requires binding to the DNA substrates, but not its ATPase dependent helicase activity to catalyze TMSD reactions. FIG. 10A. Schematic showing DNA substrates for Twinkle binding assay; FAM labeled target dsDNA (β₇γ₁₅:γ′₁₅) or PS (γ′₁₅). FIGS. 10B and 10C mitochondrial Polarization (mPolarization)c values of Twinkle-DNA complexes plotted as a function of Twinkle concentration and fitted to hyperbola. The DNA used was either a target dsDNA (FIG. 10B) or the PS (FIG. 10C). The hyperbolic fits provided the dissociation constants (K_{D}) for the equilibrium binding reactions conducted at 50 mM sodium acetate (black circles and lines). No apparent binding was observed for the reactions conducted in presence of 300 mM sodium acetate (red circles and lines). FIG. 10D. Scheme of TMSD reaction with 10 nM target dsDNA (TS, β₇γ₁₅ annealed to PS, γ′₁₅ and depicted as β₇γ₁₅:γ′₁₅) and 40 nM IS (β′₇γ′₁₅). FIGS. 10E and 10F Representative fluorescence traces from TMSD reactions conducted in presence of 50 mM sodium acetate or 300 mM sodium acetate and measured in real-time using a stopped-flow device (FIG. 10E). Reactions were either conducted in absence of Twinkle or in presence of 40 nM Twinkle hexamer. Kinetic traces were fitted to a single exponential equation to determine observed rates of the respective TMSD reactions (FIG. 10F) (Mean, N=2) FIGS. 10G and 10H. Representative fluorescence traces from spontaneous and Twinkle catalyzed TMSD reactions conducted in presence of 4 mM ATP and 10 mM magnesium acetate and measured in real-time using a stopped-flow device (FIG. 10G). A control reaction with Twinkle and ATP and magnesium acetate but with no IS added did not display unwinding of target dsDNA by Twinkle. Observed rates of these TMSD reactions obtained from fitting of the kinetic traces to an exponential equation (FIG. 10H) (Mean±SD, N=3).

FIG. 11A-11B: Catalysis of TMSD by Twinkle depends on salt concentration. FIG. 11A. Scheme of TMSD reaction with 10 nM target dsDNA (depicted as β₇γ₁₅:γ′₁₅) and 40 nM IS (β′₇γ′₁₅). 40 nM (final hexameric concentration) Twinkle was added with target dsDNA in ‘+Twinkle’ reactions. FIG. 11B. Fold change in observed rates (k_{obs}) of Twinkle catalyzed TMSD reactions over spontaneous reactions performed with 50 mM and 300 mM sodium acetate.

FIG. 12A-12J Twinkle binds to both the target dsDNA and the IS bringing them in close proximity. FIG. 12A. Schematic showing DNA substrates to measure equilibrium binding of FAM labeled target dsDNA (β₇γ₁₅:γ′₁₅) and a BHQ1 labeled 22 nucleotide dT ssDNA (BHQ1-dT₂₂) Fluorescence intensities (FIG. 12B) and fluorescence polarization (FIG. 12C) from equilibrium binding of target dsDNA and BHQ1-dT₂₂ in absence (orange) and presence (black) of Twinkle plotted as a function of BHQ1-dT₂₂ concentration. Fluorescence intensity data from ‘+Twinkle’ reactions were fitted to an inverse hyperbolic function to determine the K_{D}. The data points are the means of two independent repeats. Error associated with the K_{D} value is the standard error from the fit. FIG. 12D. Scheme of toehold hybridization reaction with FAM labeled target dsDNA (TS, β₇γ₁₅ annealed to PS, γ′₁₅ and depicted as β₇γ₁₅:γ′₁₅) and BHQ1 labeled IS lacking the branch migration domain (β′₇dT₁₅). FIGS. 12E and 12F. Representative fluorescence traces from toehold docking reactions conducted in presence and absence of 40 nM Twinkle hexamer (FIG. 12E). Kinetic traces were fitted to a single exponential equation to determine observed rates of the toehold docking reactions performed at different concentrations of IS. The observed rates thus determined were plotted as the function of IS concentration and fitted to linear function (FIG. 12F). The dotted lines represent intercepts made by the fitted straight lines on the Y-axis. (Mean±SD, N≥3). FIG. 12G-121. The slopes of linear fits in F provided ‘on rates’ (kₒₙ) for the toehold formation in presence and absence of Twinkle (FIG. 12G). The ‘off rates’ (k_{off}) for toehold dissociation were estimated from the Y-axis intercepts made by the linear fits (FIG. 12H). The ratio of k_{off}/kₒₙ provided the dissociation constants (K_{D}) for the toehold docking in absence and presence of Twinkle (FIG. 12I). The error bars are the standard errors from fitting. FIG. 12J. Free energy change (AG) accompanying toehold formation as predicted using an online tool (available on the world wide web at: arep.med.harvard.edu/cgi-bin/adnan/tm.pl) and determined using the K_{D} values depicted in FIG. 12I. Free energy difference between spontaneous and Twinkle catalyzed toehold docking is shown as ΔΔG. The errors are standard errors from the estimates.

FIG. 13A-13F: Twinkle binds to multiple DNA molecules to bring them in close proximity.

FIGS. 13A and 13B. Schematic showing DNA substrates to measure equilibrium binding of FAM labeled target dsDNA (β₇γ₁₅:γ′₁₅) (FIG. 13A) or FAM labeled single stranded TS (β₇γ₁₅) (FIG. 13B) to a BHQ1 labeled 22 nucleotide dT ssDNA (BHQ1-dT₂₂). FIGS. 13C and 13D. Fluorescence intensities from equilibrium binding of target dsDNA (FIG. 13C) or the single stranded TS (FIG. 13D) to BHQ1-dT₂₂ in absence (orange) and presence (black) of Twinkle at different BHQ1-dT₂₂ concentrations. Bars represent mean from two independent reactions. FIGS. 13E and 13F. Fluorescence polarization from equilibrium binding of target dsDNA (FIG. 13E) or the single stranded TS (FIG. 13F) to BHQ1-dT₂₂ in absence (orange) and presence (black). Bars represent mean from two independent reactions.

FIG. 14A-14G: Twinkle accelerates TMSD by catalyzing toehold formation. FIG. 14A. Scheme of TMSD reaction with target dsDNA (TS, β₇γ₁₅ annealed to PS, γ′₁₅ and depicted as β₇γ₁₅:γ′₁₅) and IS (β′₇γ′₁₅). 10 nM target dsDNA was used. 40 nM Twinkle hexamer was added to the target DNA in Twinkle catalyzed reactions. FIGS. 14B and 14C. Observed rates of spontaneous and Twinkle catalyzed TMSD reactions plotted as a function IS concentration (FIG. 14B). The spontaneous rates and the Twinkle catalyzed rates (up to 180 nM of IS) were fitted to linear trends (gray and black solid lines, respectively) to obtain bimolecular rates of TMSD (k^{TMSD}) (FIG. 14C). The complete range of observed rates of Twinkle catalyzed TMSD reactions was fitted to a hyperbola (fit shown in red, dotted line) to determine K_{M} and k_{cat}. FIG. 14D. Ratio of the bimolecular rate constants for toehold formation (kₒₙ,) and TMSD (from FIG. 14C) in absence and presence of Twinkle (kₒₙ/k^{TMSD}). FIG. 14E. Scheme of TMSD reaction with shorter toehold. The target dsDNA was kept same as in A (β₇γ₁₅:γ′₁₅). The length of the toehold domain of IS was reduced from 7 nucleotides to 3 nucleotides (β′₃γ′₁₅) to effectively shorten the toehold length from 7 to 3 base-pairs, while keeping the branch migration domain same as in FIG. 14A. FIG. 14F. Scheme of TMSD reaction with longer branch migration domain. The length of the branch migration domain was increases from 15 nucleotides to 25 nucleotides (target dsDNA, β₇γ₂₅:γ′₂₅ and IS, β′₇γ′₂₅), while keeping the toehold domain same as in FIG. 14A. FIG. 14G. Observed rates of spontaneous and catalyzed TMSD reactions conducted according to the schemes depicted in FIG. 14A (β₇γ₁₅:γ′₁₅/β′₇γ′₁₅), FIG. 14E (β₃γ₁₅:γ′₁₅/β′₇γ′₁₅) and FIG. 14F (β₇γ₂₅:γ′₂₅/β′₇γ′₂₅) (Mean±SD, N=3). Reactions were conducted with 10 nM target dsDNA, 40 nM IS and 40 nM Twinkle hexamer with target dsDNA for catalyzed reactions. All the reactions were performed using a stopped-flow device except for the spontaneous 3-nucleotide toehold reactions which were slow and was thus measured on a plate-based fluorimeter.

FIG. 15A-15G: Twinkle catalyzes TMSD reaction by accelerating the toehold formation. FIGS. 15A and 15B. Observed rates of spontaneous (FIG. 15A) and Twinkle catalyzed (FIG. 15B) TMSD reactions conducted at different invader strand concentrations. FIGS. 15C and 15D. Bimolecular rates (k, in s⁻¹·nM⁻¹) of toehold formation (association) and TMSD for reactions conducted in absence (FIG. 15C) and presence of Twinkle (FIG. 15D). FIG. 15E. Bar charts showing the observed rates of TMSD (k_{obs}) reactions with 10 nM target dsDNA (β₇γ₁₅:γ′₁₅) and 40 nM IS with 3-nucleotide toehold domain (β′₃γ′₁₅) in absence or presence of 40 nM Twinkle. Bars show the mean k_{obs} from multiple repeats (shown as black circles). Error bars represent standard deviation of the means. FIG. 15F. Bar charts showing the observed rates of spontaneous or Twinkle catalyzed TMSD (k_{obs}) reactions conducted with 10 nM target dsDNA and 40 nM IS with having either 15 nucleotides (β₇γ₁₅:γ′₁₅/β′₇γ′₁₅) or 25 nucleotides long (β₇γ₂₅:γ′₂₅/β′₇γ′₂₅) branch migration domains. Data for 15 nucleotides long branch migration domain (β₇γ₁₅:γ′₁₅/β′₇γ′₁₅) are repeated here from FIG. 10D for comparison. Bars show the mean k_{obs} from multiple repeats (shown as black circles). Error bars represent standard deviation of the means. FIG. 15G. Fold increase in observed rates of Twinkle catalyzed TMSD reactions conducted according to the schemes depicted in above for (β₇γ₁₅:γ′₁₅/β′₇γ′₁₅), (β₇γ₁₅:γ′₁₅/β′₃γ′₁₅) and (β₇γ₂₅:γ′₂₅/β′₇γ′₂₅) (Mean±SD, N=3). Reactions were conducted with 10 nM target dsDNA, 40 nM IS and 40 nM Twinkle hexamer added with target dsDNA for catalyzed reactions.

FIG. 16 The mechanistic kinetic model for Twinkle catalyzed TMSD derived from the data presented in FIGS. 9-15.

FIG. 17A-D: DNA secondary structures formed by invader strand, IS β′₆γ′₃₄ (FIG. 17A) (SEQ ID NO: 6), target strand, TS α₂₅β₆γ₃₄ (FIG. 17B) (SEQ ID NO: 4), single stranded portion of the target dsDNA, α₂₅β₆ (FIG. 17C)(SEQ ID NO: 32) and invader strand joined to the single stranded portion of target dsDNA, β′₆γ′₃₄−α₂₅β₆ (FIG. 17D)(SEQ ID NO: 33). All secondary structures were predicted on RNAfold webserver (available on the world wide web at rna.tbi.univie.ac.at/cgi-bin/RNAWebSuite/RNAfold.cgi). Predicted structures are based on minimum free energy. The horizontal color bar represents base-pair probabilities between 0 to 1. The color in the unpaired regions show the probabilities nucleotides being unpaired.

FIG. 18A-18H: Twinkle can accelerate TMSD slowed down by DNA secondary structures. FIG. 18A. TS (α₂₅β₆γ₃₄) (SEQ ID NO: 1) is shown annealed to the IS, β′₆γ′₃₄ (SEQ ID NO: 6). The nucleotide residues shown in red and blue form secondary structures within the TS and IS, respectively, and reduce the length of the toehold domain. FIG. 18B. Scheme of TMSD reaction with target dsDNA (TS, α₂₅β₆γ₃₄ annealed to PS, β′₆γ′₃₄ and depicted as β₆γ₃₄:γ′₃₄) and IS (β′₆γ′₃₄). FIG. 18C. Observed rates of spontaneous and Twinkle catalyzed TMSD reactions plotted as a function IS concentration. 10 nM target dsDNA was used in all the reactions with 40 nM Twinkle hexamer added to the target DNA in Twinkle catalyzed reactions. The spontaneous reaction rates were fitted to a linear (orange circles and line) to obtain bimolecular rates of TMSD (k^{TMSD}). Observed rates of Twinkle catalyzed reactions were fitted to a hyperbola (blue circles and line) to obtain K_{M} and k_{cat}. FIGS. 18D and 18E. Native PAGE showing kinetics of TMSD reactions performed in presence and in absence of Twinkle (FIG. 18D). For each time-point, fraction of PS displaced was quantified, plotted against time, and fitted to exponential equation to determine observed rate of TMSD (FIG. 18E). 5 nM target dsDNA and 20 nM IS was used in all the reactions. 20 nM Twinkle hexamer added with the target DNA in Twinkle catalyzed reactions. FIG. 18F. Schematic representation of the TMSD substrates with IS branch migration taking place in 5′ to 3′ direction (5′ toehold, Left panel) and 3′ to 5′ direction (3′ toehold, Right panel). The overall composition of the substrates DNA strands in terms of number and sequence of the nucleotides remained same as shown in FIG. 18B. FIG. 18G. Representative fluorescence traces showing real-time displacement of PS for the spontaneous and Twinkle catalyzed TMSD reactions conducted with substrates shown in FIG. 18F. All the reactions were performed with 40 nM IS. Kinetic traces were fitted to single exponential equation to obtained observed rate, k_{obs}. FIG. 18H. Rate of PS displacement (in fraction·min⁻¹) obtained from the TMSD reactions conducted with the substrates shown in FIG. 18F and resolved on a native PAGE. The final concentration of target dsDNA, IS and Twinkle in the reactions were 5 nM, 20 nM and 20 nM, respectively. Error bars represent the standard errors from fitting.

FIG. 19A-19H: Features of Twinkle catalyzed TMSD reactions. FIGS. 19A and 19B. Twinkle catalyzes TMSD reaction with substrates containing DNA secondary structures. Scheme of TMSD reaction with target dsDNA (TS, α₂₅β₆γ₃₄ annealed to PS, β′₆γ′₃₄ and depicted as β₆γ₃₄:γ′₃₄) and IS (β′₆γ′₃₄) (FIG. 19A). Representative fluorescence trace showing Twinkle catalyzed TMSD reaction performed with 5 nM target dsDNA, 20 nM Twinkle hexamer and 20 nM IS. Twinkle was added with target dsDNA. Reactions performed under identical conditions were also loaded on native PAGE. Fractions of PS displaced were quantified and overlaid with the stopped-flow trace (black circles) (FIG. 19B). FIGS. 19C-19F. Twinkle catalyzed strand-displacement reactions require toehold formation. Schematics depicting strand-displacement reactions in presence of IS with ‘toe’ (left panel) and in presence of IS without ‘toe’ (right panel) (FIG. 19C). Real-time traces of strand-displacement reactions with different ISs (FIG. 19D). Native PAGE showing strand displacement reactions with IS with toe (left) and with IS lacking the toe (Right) (FIG. 19E). Fraction of PS displaced plotted as a function of time and fitted to exponential equation (FIG. 19F). FIGS. 19G and 19H. Twinkle catalyzed TMSD is independent of the direction of branch migration. Schematic representation of the TMSD substrates with direction of branch migration in 5′ to 3′ direction (5′ toehold, Left panel) and 3′ to 5′ direction (3′ toehold, Right panel) (FIG. 19G). Fractions of PS displaced from gel-based assay plotted as a function of time and fitted to exponential trend.

FIG. 20A-20F: fine tuning the rate of Twinkle catalyzed TMSD. FIGS. 20A-20E. Schematic representation of different substrate designs used for conducting Twinkle catalyzed TMSD reactions. FIG. 20F. Observed rates of catalyzed TMSD reactions determined using real-time stopped flow-assay (Mean±SD, N≥3). Final concentrations of the target dsDNA, IS and Twinkle in the reactions were 10 nM, 40 nM and 40 nM, respectively.

FIG. 21A-21F: Twinkle catalyzed TMSD reactions are sensitive to nucleotide mismatches. FIG. 21A. Substrate design to study effect of nucleotide mismatches on Twinkle catalyzed TMSD rates fluorescence-based assay. FIG. 21B. Observed rates of Twinkle catalyzed TMSD reactions with ISs consisting of 0, 1 or 2 nucleotide mismatch(es). The rate of the reaction with IS having 3 nucleotide mismatches was very slow and could not be fitted to exponential trend. The error bars represent standard errors from fitting. FIG. 21C. Design of the TMSD substrates to test the effect of nucleotide mismatches on Twinkle catalyzed TMSD reactions using a gel-based assay. The 5′-end of the PS was radiolabeled with γ³²P. FIGS. 21D-21F. Native PAGE analysis of the displacement of radiolabeled PS. Reactions were performed according to the scheme depicted in C with the IS containing 0, 1 or 3 nucleotide mismatch(es) (FIG. 21D). Final concentrations of the target dsDNA, IS and Twinkle in the reactions were 2 nM, 20 nM and 10 nM, respectively. Fraction of PS displaced for each time point obtained from gel-based assays, plotted against time and fitted to exponential equation (FIG. 21E). Bar chart showing the initial rates of PS displacement (in fraction·min⁻¹) for the reactions conducted with stated substrates (FIG. 21F). No apparent PS displacement was observed in the reaction with IS containing 3 nucleotide mismatches.

### DETAILED DESCRIPTION

DNA nanotechnology often utilizes ‘toeholds,’ which are small single-stranded DNA overhangs, in kinetically controlled complex biochemical circuits for designing nano-devices. We have previously reported that human mitochondrial DNA helicase Twinkle possesses an unexpected DNA annealing activity and can catalyze helicase-coupled homologous DNA strand exchange reactions. Here, we demonstrate that Twinkle can catalyze TMSD on various DNA substrates. In contrast to the homologous DNA strand exchange, which is driven by Twinkle's active helicase activity, the observed increase in the rate of Twinkle-catalyzed TMSD is independent of the helicase activity. The Twinkle catalyzed TMSD can be kinetically modulated by changing the length of external or internal toehold domains, therefore providing additional tunability and control over reaction outcomes. Furthermore, we show that the Twinkle catalyzed strand displacement discriminates single base changes, and thus, can be utilized for developing diagnostic probes for the detection of single nucleotide polymorphisms.

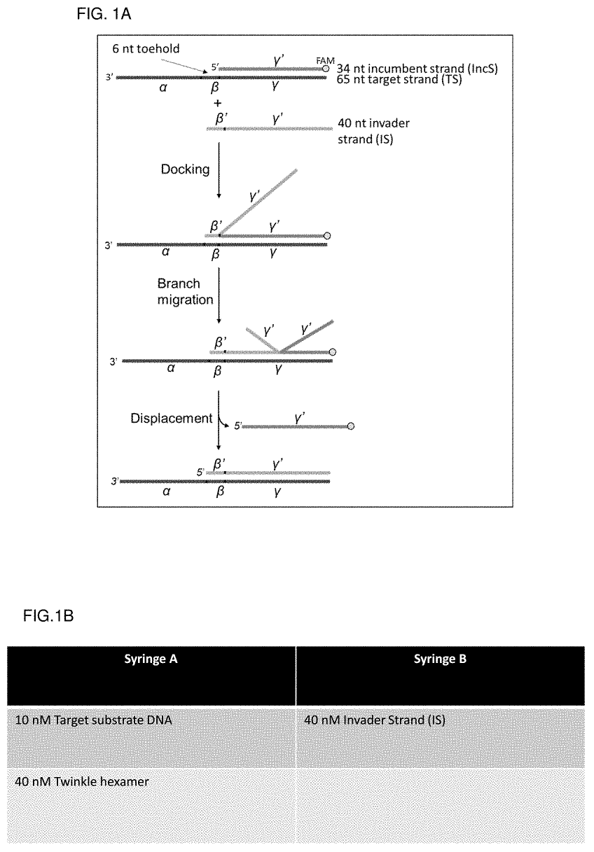

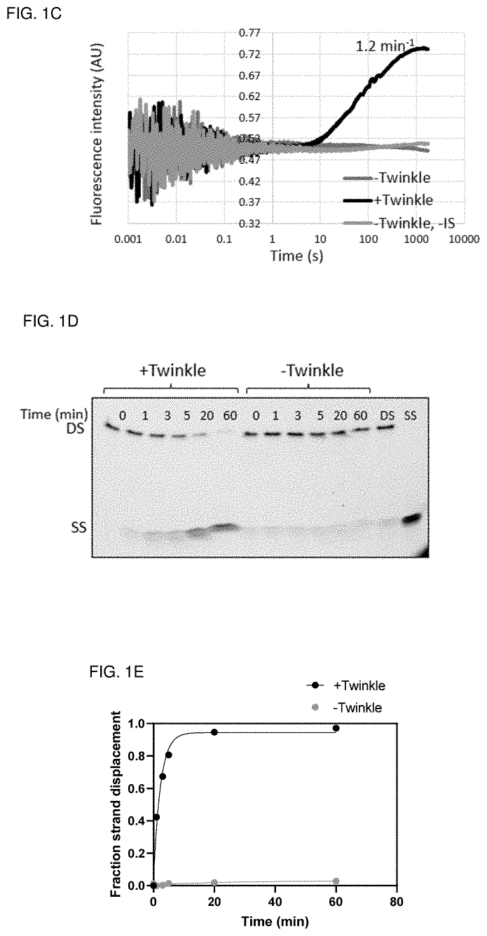

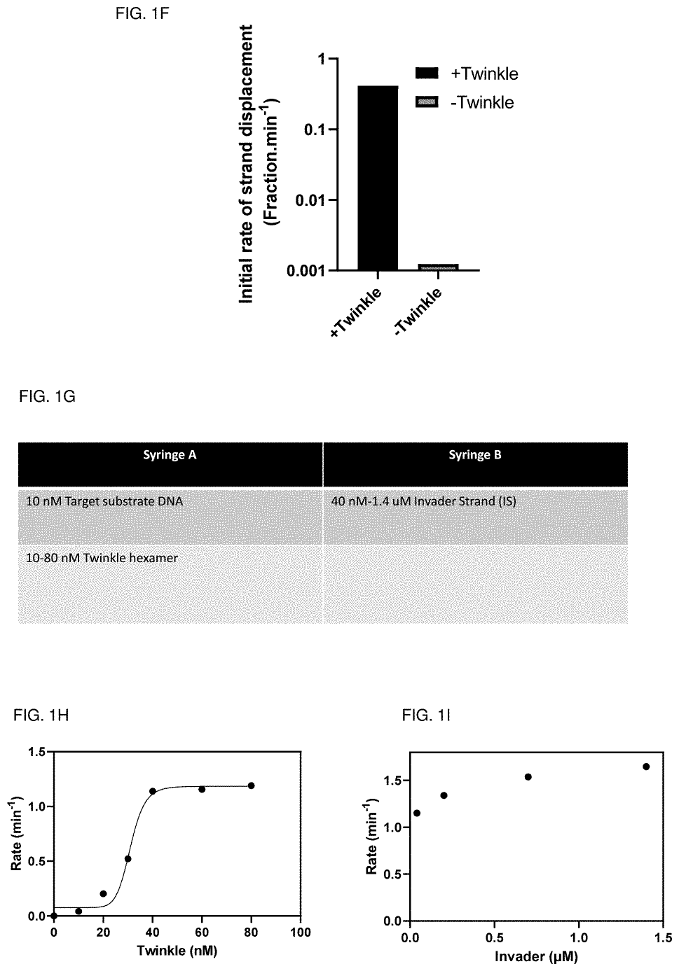

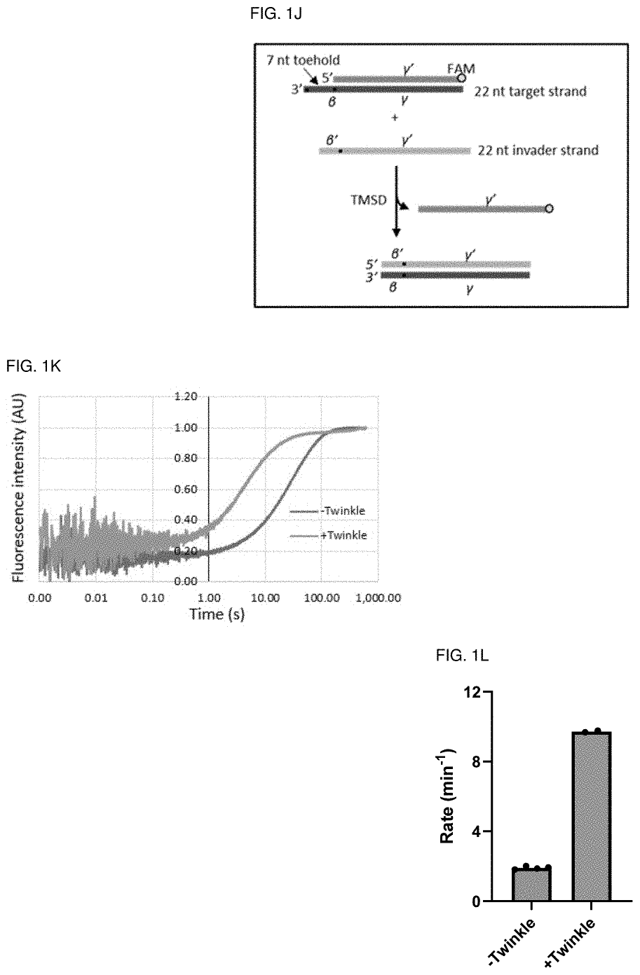

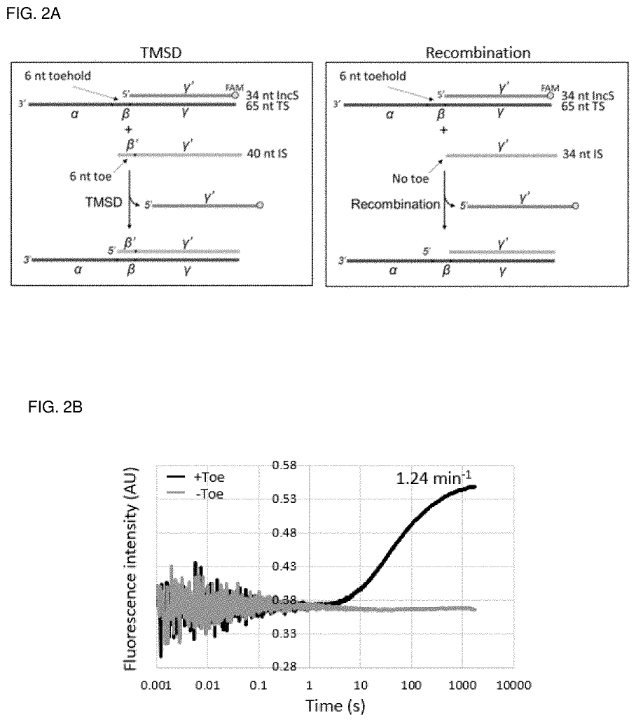

Human mitochondrial helicase Twinkle is an essential component of mitochondrial DNA replication. In addition to its unwinding activity, we have previously demonstrated that it has annealing activity and can catalyze annealing of two single strands of complementary DNA (1). Moreover, Twinkle can couple its annealing activity with the unwinding activity to perform strand exchange between the unwound DNA strand and a homologous DNA strand. Twinkle can catalyze branch migration and resolution of a four-way DNA junction (2). Interestingly, unlike the annealing activity, Twinkle requires nucleotide hydrolysis for strand exchange reactions. Single-stranded DNA ‘toeholds’ are used to kinetically control DNA strand displacement reactions (3). TMSD is an important tool in DNA nanotechnology and is widely used to construct DNA nano-circuits and devices (4). Typical steps in a TMSD reaction are the binding of the toehold to complementary region of the invader strand (docking), branch migration, and subsequent displacement of the incumbent strand by the invader strand (see the scheme in FIG. 1A). There has been a great interest in devising novel approaches to control the kinetics of TMSD.

Twinkle accelerates the TMSD reactions up to 1000-fold by positioning the single stranded toehold domains of TMSD substrates in a ‘Jencksian circe’ to catalyze toehold docking. In addition to the primary site in the central channel for tight DNA binding, accessary binding sites confer Twinkle with the ability to bind multiple DNA molecules. Binding energy from these events compensates for the thermodynamic barrier encountered by enzyme-free DNA substrates. The data provided herein indicates that Twinkle drives the catalysis solely by accelerating toehold formation without affecting the rates of branch migration step. Furthermore, the catalysis follows typical Briggs-Haldane enzyme kinetics, which is used to determine quantitative parameters crucial to design catalyzed TMSD reactions.

The rates of Twinkle-catalyzed TMSD reactions can be modulated by changes in toehold domain or branch migration domain of target strand (TS), invader strand (IS), and protector strand (PS). The Twinkle-catalyzed rates are also sensitive to mismatches in the branch migration domain of TS and IS. While rapid response times and regulation are advantageous features for leveraging the catalyzed TMSD reaction for broader applications in the field of DNA nanotechnology, our results also imply Twinkle as a potential player in human mitochondrial DNA recombination, repair and deletion.

As the TMSD reactions involve DNA annealing and branch migration, we hypothesized that Twinkle, owing to its annealing and strand exchange activities, could catalyze these reactions. We employed fluorescence based stopped-flow kinetics and electrophoretic methods and demonstrated that variants of Twinkle C-terminal domain increase the rate of displacement between 10-1000 fold.

### Definitions

The following are provided to facilitate the practice of the invention. They are not intended to limit the invention in any way.

Twinkle is a hexameric DNA helicase which unwinds short stretches of double-stranded DNA in the 5′ to 3′ direction and, along with mitochondrial single-stranded DNA binding protein and mtDNA polymerase gamma which plays a key role in mtDNA replication. The protein localizes to the mitochondrial matrix and mitochondrial nucleoids. Mutations in this gene cause infantile onset spinocerebellar ataxia (IOSCA) and progressive external ophthalmoplegia (PEO) and are also associated with several mitochondrial depletion syndromes. Alternative splicing results in multiple transcript variants encoding distinct isoforms. The nucleic acid and protein sequences are in the public domain and can be found at HGNC: 1160 NCBI Entrez Gene: 56652 Ensembl: ENSG00000107815, OMIM®: 606075, and UniProtKB/Swiss-Prot: Q96RR1.

The phrase “truncated variant” when used in reference to the Twinkle CTD described herein refers to a molecule wherein between 10-100, 40-200, 10-300, 43-359, 1-359 amino acids are deleted from the N terminal end of Twinkle. In certain aspects, the entirety of the N-terminal domain (amino acids 43-359) may be deleted. Nucleic acids encoding the truncated CTD variants described above are also included and are further described below.

The term “nucleic acid” refers to natural nucleic acids, artificial nucleic acids, analogs thereof, or combinations thereof. Nucleic acids may also include analogs of DNA or RNA having modifications to either the bases or the backbone. For example, nucleic acid, as used herein, can include the use of peptide nucleic acids (PNA). The term “nucleic acids” also includes chimeric molecules.

By “nucleic acid sequence” is meant a nucleic acid which comprises an individual sequence. When a first, second, or third nucleic acid sequence is referred to, this is meant that the individual nucleotides of each of the first, second, third, etc., nucleic acid sequence are unique and differ from each other. In other words, the first nucleic acid sequence will differ in nucleotide sequences from the second and third, etc. There can be multiple nucleic acid sequences with the same sequence. For instance, when a “first nucleic acid sequence” is referred to, this can include multiple copies of the same sequence, all of which are referred to as a “first nucleic acid sequence.”

Typically, at least two different nucleic acid sequences are used in self-assembly pathways, although three, four, five, six or more may be used. Typically, each nucleic acid sequence comprises at least one domain that is complementary to at least a portion of one other sequence being used for the self-assembly pathway. Individual nucleic acid sequences are discussed in more detail below.

The term “toehold” refers to an overhang nucleic acid sequence designed to initiate hybridization of the domain with a complementary nucleic acid sequence. The secondary structure of a nucleic acid sequence may be such that the toehold is exposed or sequestered. For example, in some embodiments, the secondary structure of the toehold is such that the toehold is available to hybridize to a complementary nucleic acid (the toehold is “exposed,” or “accessible”), and in other embodiments, the secondary structure of the toehold is such that the toehold is not available to hybridize to a complementary nucleic acid (the toehold is “sequestered,” or “inaccessible”). If the toehold is sequestered or otherwise unavailable, the toehold can be made available by some event such as, for example, the opening of the hairpin of which it is a part of. When exposed, a toehold is configured such that a complementary nucleic acid sequence can hybridize at the toehold.

The term “oligonucleotides,” or “oligos” as used herein refers to oligomers of natural (RNA or DNA) or modified nucleic acid sequences or linkages, including natural and unnatural deoxyribonucleotides, ribonucleotides, anomeric forms thereof, PNAs, locked nucleotide acids monomers (LNA), and the like and/or combinations thereof, capable of specifically binding to a single-stranded polynucleotide by way of a regular pattern of sequence-to-sequence interactions, such as Watson-Crick type of base pairing, base stacking, Hoogsteen or reverse Hoogsteen types of base pairing, or the like. Usually, nucleic acid sequences are linked by phosphodiester bonds or analogs thereof to form oligonucleotides ranging in size from a few base units, e.g., 8-12, to several tens of base units, e.g., 100-200. Suitable oligonucleotides may be prepared by the phosphoramidite method described by Beaucage and Carruthers (Tetrahedron Lett., 22, 1859-1862, 1981), or by the triester method according to Matteucci, et al. (J. Am. Chem. Soc., 103, 3185, 1981), both incorporated herein by reference, or by other chemical methods such as using a commercial automated oligonucleotide synthesizer. Oligonucleotides (both DNA and RNA) may also be synthesized enzymatically for instance by transcription or strand displacement amplification. Typically, oligonucleotides are single-stranded, but double-stranded or partially double-stranded oligos may also be used in certain embodiments of the invention. An “oligo pair” is a pair of oligos that specifically bind to one another (i.e., are complementary (e.g., perfectly complementary) to one another).

The terms “complementary” and “complementarity” refer to oligonucleotides related by base-pairing rules. Complementary nucleotides are, generally, A and T (or A and U), or C and G. For example, for the sequence “5′-AGT-3′,” the perfectly complementary sequence is “3′-TCA-5′.” Methods for calculating the level of complementarity between two nucleic acids are widely known to those of ordinary skill in the art. For example, complementarity may be computed using online resources, such as, e.g., the NCBI BLAST website (ncbi.nlm.nih.gov/blast/producttable.shtml) and the Oligonucleotides Properties Calculator on the Northwestern University website (basic.northwestem.edu/biotools/oligocalc.html). Two single-stranded RNA or DNA molecules may be considered substantially complementary when the nucleotides of one strand, optimally aligned and with appropriate nucleotide insertions or deletions, pair with at least about 80% of the nucleotides of the other strand, usually at least about 90% to 95%, and more preferably from about 98 to 100%. Two single-stranded oligonucleotides are considered perfectly complementary when the nucleotides of one strand, optimally aligned and with appropriate nucleotide insertions or deletions, pair with 100% of the nucleotides of the other strand. Alternatively, substantial complementarity exists when a first oligonucleotide will hybridize under selective hybridization conditions to a second oligonucleotide. Selective hybridization conditions include, but are not limited to, stringent hybridization conditions. Selective hybridization, or substantially complementary hybridization, occurs when at least about 65% of the nucleic acid sequences within a first oligonucleotide over a stretch of at least 14 to 25 sequences pair with a perfectly complementary sequences within a second oligonucleotide, preferably at least about 75%, more preferably at least about 90%. Preferably, the two nucleic acid sequences have at least 95%, 96%, 97%, 98%, 99% or 100% of sequence identity. See, M. Kanehisa, Nucleic Acids Res. 12, 203 (1984), incorporated herein by reference. For shorter nucleotide sequences selective hybridization occurs when at least about 65% of the nucleic acid sequences within a first oligonucleotide over a stretch of at least 8 to 12 nucleotides pair with a perfectly complementary nucleic acid sequence within a second oligonucleotide, preferably at least about 75%, more preferably at least about 90%. Stringent hybridization conditions will typically include salt concentrations of less than about 1 M, more usually less than about 500 mM and preferably less than about 200 mM. Hybridization temperatures can be as low as 5° C., and are preferably lower than about 30° C. However, longer fragments may require higher hybridization temperatures for specific hybridization. Hybridization temperatures are generally at least about 2° C. to 6° C. lower than melting temperatures (Tm), which are defined below.

As used herein, “two perfectly matched nucleotide sequences” refers to a nucleic acid duplex wherein the two nucleotide strands match according to the Watson-Crick basepair principle, i.e., A-T and C-G pairs in DNA:DNA duplex and A-U and C-G pairs in DNA:RNA or RNA:RNA duplex, and there is no deletion or addition in each of the two strands.

The term, “mismatch” refers to a nucleic acid duplex wherein at least one of the nucleotide base pairs do not form a match according to the Watson-Crick basepair principle. For example, A-C or U-G “pairs” are lined up, which are not capable of forming a basepair. The mismatch can be in a single set of bases, or in two, three, four, five, or more basepairs of the nucleic acid duplex.

As used herein, “complementary to each other over at least a portion of their sequence” means that at least two or more consecutive nucleotide base pairs are complementary to each other. For example, 3, 4, 5, 6, 7, 8, 9, 10, 11, 12, 13, 14, 15, 16, 17, 18, 19, 20, 21, 22, 23, 24, 25, 26, 27, 28, 29, 30, or more consecutive nucleotide base pairs can be complementary to each other over the length of the nucleic acid sequence.

As used herein, “substantially hybridized” refers to the conditions under which a stable duplex is formed between two nucleic acid sequences, and can be detected. This is discussed in more detail below.

The term “enzyme-assisted” as used herein is defined to mean any chemical process where a protein or other chemical entity is used to catalyze or increase the rate of a chemical reaction. The protein used for this purpose can include, but is not limited to, chains of amino acids (natural or unnatural), that may or may not contain other chemical variations and can have a defined secondary structure. The chemical reaction can include, but is not limited to, reactions of RNA or portions of RNA, DNA or portions of DNA, and/or any nucleotide or derivative thereof. Typically, enzymes catalyze reactions through binding to specific or non-specific target molecular portions usually indicated as binding sites.

As used herein, “melting temperature” (“Tm”) refers to the midpoint of the temperature range over which nucleic acid duplex, i.e., DNA:DNA, DNA:RNA and RNA:RNA, is denatured.

As used herein: “stringency of hybridization” in determining percentage mismatch is as follows:
1) high stringency: 0.1×SSPE, 0.1% SDS, 65° C.;
2) medium stringency: 0.2×SSPE, 0.1% SDS, 50° C. (also referred to as moderate stringency); and
3) low stringency: 1.0×SSPE, 0.1% SDS, 50° C.

It is understood that equivalent stringencies may be achieved using alternative buffers, salts and temperatures (See generally, Ausubel (Ed.) Current Protocols in Molecular Biology, 2.9A. Southern Blotting, 2.9B. Dot and Slot Blotting of DNA and 2.10. Hybridization Analysis of DNA Blots, John Wiley & Sons, Inc. (2000)).

As used herein, a “significant reduction in background hybridization” means that non-specific hybridization, or hybridization between unintended nucleic acid sequences, is reduced by at least 80%, more preferably by at least 90%, even more preferably by at least 95%, still more preferably by at least 99%.

By “preferentially binds” it is meant that a specific binding event between a first and second molecule occurs at least 20 times or more, preferably 50 times or more, more preferably 100 times or more, and even 1000 times or more often than a nonspecific binding event between the first molecule and a molecule that is not the second molecule. For example, a capture moiety can be designed to preferentially bind to a given target agent at least 20 times or more, preferably 50 times or more, more preferably 100 times or more, and even 1000 times or more often than to other molecules in a biological solution. Also, an immobilized binding partner, in certain embodiments, will preferentially bind to a target agent, capture moiety, or capture moiety/target agent complex.

The term “sample” in the present specification and claims is used in its broadest sense and can be, by non-limiting example, any sample that is suspected of containing a target agent(s) to be detected. It is meant to include specimens or cultures (e.g., microbiological cultures), and biological and environmental specimens as well as non-biological specimens. Biological samples may comprise animal-derived materials, including fluid (e.g., blood, saliva, urine, lymph, etc.), solid (e.g., stool) or tissue (e.g., buccal, organ-specific, skin, etc.), as well as liquid and solid food and feed products and ingredients such as dairy items, vegetables, meat and meat by-products, and waste. Biological samples may be obtained from, e.g., humans, any domestic or wild animals, plants, bacteria or other microorganisms, etc. Environmental samples can include environmental material such as surface matter, soil, water (e.g., contaminated water), air and industrial samples, as well as samples obtained from food and dairy processing instruments, apparatus, equipment, utensils, disposable and non-disposable items. These examples are not to be construed as limiting the sample types applicable to the present invention. Those of skill in the art would appreciate and understand the particular type of sample required for the detection of particular target agents (Pawliszyn, J., Sampling and Sample Preparation for Field and Laboratory, (2002); Venkatesh Iyengar, G., et al., Element Analysis of Biological Samples: Principles and Practices (1998); Drielak, S., Hot Zone Forensics: Chemical, Biological, and Radiological Evidence Collection (2004); and Nielsen, D. M., Practical Handbook of Environmental Site Characterization and Ground-Water Monitoring (2005)).

As used herein, “a promoter, a promoter region or promoter element” refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated.

As used herein, “operatively linked or operationally associated” refers to the functional relationship of nucleic acids with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it may be necessary to remove, add or alter 5′ untranslated portions of the clones to eliminate extra, potential inappropriate alternative translation initiation (i.e., start) codons or other sequences that may interfere with or reduce expression, either at the level of transcription or translation. Alternatively, consensus ribosome binding sites (see, e.g., Kozak, J. Biol. Chem., 266:19867-19870 (1991)) can be inserted immediately 5′ of the start codon and may enhance expression. The desirability of (or need for) such modification may be empirically determined.

As used herein, “RNA polymerase” refers to an enzyme that synthesizes RNA using a DNA or RNA as the template. It is intended to encompass any RNA polymerase with conservative amino acid substitutions that do not substantially alter its activity.

As used herein, “reverse transcriptase” refers to an enzyme that synthesizes DNA using a RNA as the template. It is intended to encompass any reverse transcriptase with conservative amino acid substitutions that do not substantially alter its activity.

### Uses

The methods and devices disclosed herein can be used for multiple applications. Detection and identification of virtually any nucleic acid sequence, or non-nucleic acid sequence, can be accomplished. For example, the presence of specific viruses, microorganisms and parasites can be detected. Genetic diseases can also be detected and diagnosed, either by detection of sequence variations (mutations) which cause or are associated with a disease or are linked (Restriction Fragment Length Polymorphisms or RFLP's) to the disease locus. Sequence variations which are associated with, or cause, cancer, can also be detected. This can allow for both the diagnosis and prognosis of disease. For example, if a breast cancer marker is detected in an individual, the individual can be made aware of their increased likelihood of developing breast cancer, and can be treated accordingly. The methods and devices disclosed herein can also be used in the detection and identification of nucleic acid sequences for forensic fingerprinting, tissue typing and for taxonomic purposes, namely the identification and speciation of microorganisms, flora and fauna.

Accordingly, the methods and devices disclosed herein have applications in clinical medicine, veterinary science, aquaculture, horticulture and agriculture.

### Kits

The compositions described herein can be used to advantage in a kit for conducting TMSD reactions. An exemplary kit comprises a CTD truncated variant as described herein. In a preferred embodiment, the CTD variant comprises a sequence tag to facilitate purification and/or solubility. In a preferred embodiment, the tag is a SUMO tag. The kit may also comprise buffers suitable for conducting TMSD reactions. Positive and negative control nucleic acid constructs may optionally be included in the kit.

The materials and methods set forth below are provided to facilitate practice of the invention. They are not intended to limit the invention in any way.

### Oligonucleotides:

All oligonucleotides used in the study were ordered HPLC purified from IDT. The sequences of the oligos used are provided in Table 1.

TABLE 1
Nucleotide sequences of oligonucleotides used in the study.

| | | | |
|---|---|---|---|
| | | | SEQ |
| Oligo | Alternate | | ID |
| ID | Oligo ID | Sequence | NO |

Long DNA substrates: 5′ Toehold

| | | | |
|---|---|---|---|
| TS 65 | TS α₂₅ β₆ γ₃₄ | 5′ GGGGAGATGAGTCACGAGAGAGTCTTGTGATG | 1 |
| | | CTCCTACGTAGTTGAATCTCTTCCACTAACCAGCGC 3′ | |
| IncS 34 | PS γ′₃₄ | 5′ AGGAGCATCACAAGACTCTCTCGTGACTCATCTC 3′ | 2 |
| IS 40 | IS β′₆ γ′₃₄ | 5′ C TAC GTA GGA GCA TCA CAA GAC TCT CTC GTG ACT | 3 |
| | | CAT CTC 3′ | |
| | IS β′₆ γ′₃₄ | 5′ CTACGTAGGAGCATCACTAGACTCTC | 23 |
| | 1 nt | TCGTGACTCATCTC 3′ | |
| | mismatch | | |
| | IS β′₆ γ′₃₄ | 5′ CTACGTAGGAGCATCACTTCACTCTC | 24 |
| | 3 nt | TCGTGACTCATCTC 3′ | |
| | mismatch | | |
| TS40 | TS β₆γ₃₄ | 5′ GGGGAGATGAGTCACGAGAGAGTCTTGTGA | 10 |
| | | TGCTCCTACGTAG 3′ | |
| No | | 5′ AGGAGCATCACAAGACTCTCTCGTGACTCATCTC 3′ | 11 |
| toehold | | | |
| IS34 | | | |
| P25 | α′₂₅ | 5′ GCGCTGGTTAGTGGAAGAGATTCAA 3′ | 12 |
| | α′₂₄ | 5′ GCGCTGGTTAGTGGAAGAGATTCA 3 | 25 |
| IS40- | IS | 5′ TTTTTTTTTTTTTTTTTTTTTTTTTTTTTTTTTTTCTACGT | 13 |
| dT35 | dT₃₅β′₆γ′₃₄ | AGGAGCATCACAAGACTCTCTCGTGACTCATCTC 3′ | |
| IS40- | IS | 5′ TTTTTTTTTTTTTTTTTTTTTTTTTTTTTTTTTTTCTACGT | 14 |
| dT35 | dT₃₅β′₆γ′₃₄ | AGGAGCATCACTAGACTCTCTCGTGACTCATCTC 3′ | |
| 1 nt | 1 nt | | |
| mismatch | mismatch | | |
| IS40- | IS | 5′ TTTTTTTTTTTTTTTTTTTTTTTTTTTTTTTTTTTCTACGT | 15 |
| dT35 | d′T₃₅β′₆γ′₃₄ | AGGAGCATCACTTGACTCTCTCGTGACTCATCTC 3′ | |
| 2 nt | 2 nt | | |
| mismatch | mismatch | | |
| IS40- | IS | 5′ TTTTTTTTTTTTTTTTTTTTTTTTTTTTTTTTTTTCTACGT | 16 |
| dT35 | dT₃₅β′₆γ′₃₄ | AGGAGCATCACTTCACTCTCTCGTGACTCATCTC 3′ | |
| 3 nt | 3 nt | | |
| mismatch | mismatch | | |

Long DNA substrates: 3′ Toehold

| | | | |
|---|---|---|---|
| TS65 | TS α₂₅ | 5′ CGCGACCAATCACCTTCTCTAAGTTGATGCATCC | 4 |
| | β₆ γ₃₄ | TCGTAGTGTTCTGAGAGAGCACTGAGTAGAGGGG 3′ | |
| IncS 34 | PS γ′₃₄ | 5′ FAM-CTCTACTCAGTGCTCTCTCAGAACACTACGAGGA | 5 |
| | | 3′ | |
| TS 40 | IS β′₆ γ′₃₄ | 5′ | 6 |
| | | CTCTACTCAGTGCTCTCTCAGAACACTACGAGGATGCATC | |
| | | 3′ | |

Short DNA substrates

| | | | |
|---|---|---|---|
| TS 25 | TS β₇ γ₁₅ | 5′ GTGGAGACGTAGGGTATTGAAGGGG 3′ | 7 |
| InC 15 | PS γ₁₅ | 5′ CTTCAATACCCTACG 3′ | 8 |
| IS 22 | IS β′₇γ′₁₅ | 5′ CTTCAATACCCTACGTCTCCAC 3′ | 9 |
| | dT₂₂ | 5′ TTTTTTTTTTTTTTTTTTTTTT 3′ | 26 |
| | β′₇dT₁₅ | 5′ CTTCAATTTTTTTT 3′ | 27 |
| | IS β′₃ γ′₁₅ | 5′ AATACCCTACGTCTCCAC 3′ | 28 |
| | TS β₇ γ₂₅ | 5′ GTGGAGACGTAGGGTATTGAGATGAACGAGAGGGG 3′ | 29 |
| | PS γ′₂₅ | 5′ CTCTCGTTCATCTCAATACCCTACG 3′ | 30 |
| | IS β′₇ γ′₂₅ | 5′ CTCTCGTTCATCTCAATACCCTACGTCTCCAC 3′ | 31 |

Oligos were dissolved in 1×TE buffer and their concentrations were determined. Target substrate DNA was made by annealing target strand (TS) and incumbent strand (IncS) in annealing buffer (1×TE, 100 mM NaCl). Briefly, the oligo mixture was heated at 95° C. for 2 minutes on a heat block and was allowed to gradually cool down to room temperature. Annealing of TS and IncS was checked on 20% native TBE gel.

### Real-Time Stopped-Flow Assay:

Strand displacement was monitored in real-time by means of a stopped-flow assay. Changes in fluorescence signal was measured in millisecond to minutes time range. Syringe A of the stopped flow apparatus was filled with either 20 nM target dsDNA (annealed TS:PS) alone or with 20 nM target dsDNA and 80 nM Twinkle (hexameric concentration) and Syringe B was filled with 80 nM IS. Both the solutions were made in 1×reaction buffer (50 mM Tris-acetate, pH 7.5, 50 mM sodium acetate, 0.01% Tween 20, 1 mM EDTA and 5 mM DTT). Rapid mixing was performed, and fluorescence signal was monitored in the flow cell. The displacement of IncS (PS) by IS and its subsequent release resulted in increase in fluorescence signal with time. Fluorescence traces were fitted to either 1-exponential, Fl=A(1−e^{-kt}), or 2-exponential trends to achieve fits with minimized standard errors. The rate of the faster (and the major) phase was reported as the rate of strand displacement. All the experiments were performed at stopped-flow chamber set at 25° C. Target substrate DNA was incubated with Twinkle for 20 minutes at 25° C. before triggering the reactions. The final concentration of the components after mixing was 10 nM Target substrate DNA, 40 nM IS and 40 nM Twinkle hexamer.

Slight variations in the above strategy were made to conduct different reactions. For determining Twinkle concentration dependence, 10 nM to 80 nM final concentration of Twinkle hexamer was used. For testing the effect of IS concentration, 40 nM to 1.4 uM IS was used while keeping target substrate DNA and Twinkle concentrations 10 nM and 40 nM, respectively. For reactions conducted with magnesium acetate and ATP, 11 mM magnesium acetate was added in both the syringes while 4 mM ATP was added to Syringe 2.

### Gel-Based Assay to Study Strand Displacement Reactions:

To directly observe the displaced InS (PS), a discontinuous gel-based assay was used. Two mixtures were prepared with Mixture A consisting of 10 nM target substrate DNA (annealed TS-IncS), 40 nM Twinkle hexamer (where mentioned) and Mixture B containing 40 nM IS. Both the mixtures were prepared in 1×reaction buffer (50 mM Tris-acetate, pH 7.5, 50 mM sodium acetate, 0.01% Tween 20, 1 mM EDTA and 5 mM DTT). Mixtures were pre-incubated for 20 minutes before initiating of reactions.

In experiments with magnesium acetate and ATP, 11 mM magnesium acetate was added to both Mixtures A and B and 4 mM ATP was added to Mixture B. Reactions were initiated by mixing equal volumes of Mixtures A and B. Equal volumes of reaction mixture were drawn at stated time-points, mixed with 0.5% SDS and immediately loaded on 20% TBE native gel continuously running at low voltage of 30 V with 1×TBE buffer. Once all the time-points were loaded, the gel was run at 120 V for around 2 hours to resolve the substrate and product DNAs. The gels were run with cold buffer while keeping the gel apparatus on ice. Gels were scanned using GE Typhoon 9500 imaging system and fluorescence intensity of target substrate DNA (doubled stranded, DS) and displaced IncS (single stranded, SS) were quantified. Fractions of IncS (SS DNA) were determined for each reaction time (t) and were plotted as a function of time. The data were fitted in exponential equation (equation 1) which provided first order rate constant of strand displacement reaction (k), maximum amplitude (maximum fraction of IncS displaced, A) and residual standard errors in the determination of k and A.

Fr=A(1−e^{-kt}) (Equation 1)

Initial rate of strand displacement was determined as:

Initial rate=A·k (Equation 2)

Standard error of initial rate was determined as:

S.E.=(k×SE_{A}²+A×SEₖ²)^{1/2}

Where k is first-order rate constant, A is maximum amplitude, SE_{A} is standard error amplitude and SEₖ is standard error of rate.

### Binding Assay for Twinkle-DNA Binding:

Fluorescence anisotropy measurements were used to determine the dissociation constants for Twinkle binding to either a Fam labeled ssDNA or dsDNA. Briefly, 5 nM DNA or DNA were incubated with a range of different concentrations of Twinkle in TMSD buffer consisting of or in 300 mM sodium acetate buffer. Equilibrium binding was detected by measuring change in fluorescence polarization (p) as the twinkle concentration, [T] was increased. For the binding reactions performed with 50 mM salt, the polarization values were fitted to hyperbola to determine K_{D}.

$$Y = \frac{{p}_{max} * [ T ]}{( {K}_{D} + [ T ] )} + {Y}_{0} \qquad ( Equation 3 )$$

The reactions made at 300 mM salt did not result in remarkable polarization change and couldn't be fitted to the equation with high confidence.

### Observation of the Accessory Binding Sites:

For determination of dissociation constant for the Twinkle-target dsDNA-BHQ1-dT22 DNA, we incubated 40 nM of target dsDNA and 40 nM of Twinkle at 25° C. for 20 mins. Increasing concentrations of BHQ1-dT22 DNA were added to the complex and allowed to incubate for further 10 mins. Fluorescence intensity and polarization for each sample were measured on a Tecan Spark plate-based fluorimeter at 25° C. The decreasing fluorescence was fitted to an inverse hyperbola to determine K_{D} for the complex. In ‘-Twinkle’ reactions, no twinkle was added. Similarly, the reactions were performed with ssDNA, rather than using target dsDNA.

### Measurement of the Product Off-Rate:

The off-rate for the dissociation of the TMSD product (IS:TS) and Twinkle was measured using a stopped flow fluorescence assay. Briefly, 80 nM Twinkle (hexameric concentration) and 20 nM FAM labeled TMSD product (FAM-IS:TS) were incubated for 20 minutes at 25° C., and were loaded in syringe A. Similarly, 10-fold excess of unlabeled product DNA (200 nM of IS:TS) was incubated at 25° C. for 20 minutes and loaded in syringe B. Equal volumes of the contents in the two syringes were rapidly mixed and fluorescence intensity of the reaction was measured in the flow-cell. As the FAM-labeled product falls off the Twinkle, the fluorescence intensity decreases slightly. The excess of unlabeled product DNA traps the available Twinkle binding sites to prevent rebinding labeled DNA to Twinkle. Fluorescence traces were fitted to 1-exponential, Fl=A(1−e^{-kt}) to achieve fits with minimized standard errors. The experiments were performed with the stopped-flow chamber set at 25° C.

The following Examples are provided to illustrate certain embodiments of the invention. They are not intended to limit the invention in any way.

### Example I

### Twinkle Catalyzes TMSD Reactions

A TMSD reaction was designed with the 3′ end of the incumbent strand (IncS) labeled with a FAM (6-FAM, Fluorescein derivative) moiety while the 5′ end of the target strand (TS) had four dG residues (FIG. 1A). When the two strands (IncS and TS) are properly annealed to form a target DNA substrate, the four dG residues (three of which form a 5′ overhang while one remains annealed to the IncS) quench the FAM fluorescence. The fluorescence signal from FAM was expected to increase when IncS was completely displaced by the invader strand (IS). The duplex region formed by annealed target DNA substrate (IncS and TS) was 34 nucleotides. The TS contained a 31 nucleotide long 3′ ssDNA overhang, of which the six nucleotides proximal to the annealed IncS formed a ‘toehold’ to dock six nucleotide ‘toe’ of the 40 nucleotide long IS. This substrate was used to measure precise kinetics of TMSD reactions using a stopped-flow fluorescence assay. Briefly, one of the stopped-flow syringes contained target substrate DNA with or without Twinkle. To start the reaction, IS was rapidly mixed from the other syringe and fluorescence intensity was measured in milliseconds to minutes range (FIG. 1B).

No change in the baseline signal in absence of Twinkle was observed, suggesting no significant strand displacement. The presence of Twinkle increased the amplitude and rate of the fluorescence signal, showing Twinkle's ability to catalyze the strand displacement on a long IncS-TS duplex DNA (FIG. 1C). The control reactions lacking IS either in presence or absence of Twinkle did not produce observable change in the fluorescence signal showing that IS is necessary for the separation of the IncS and TS, and the fluorescence signal increase is not the consequence of Twinkle unwinding the target DNA substrate. Fitting of the kinetic data in double exponential kinetic model provided the rate of strand displacement reaction to be 1.2 min⁻¹ (FIG. 1C).

One way to confirm that Twinkle is catalyzing TMSD reactions is to directly detect the strand-displaced DNA product (free IncS). We visualized the reactions on a native gel by loading TMSD reactions conducted for different time periods. As there is no method to quench these reactions, we loaded the reactions on a gel while running it at low voltage of 30 V, such that electrophoretic movement would separate the displaced product (IncS) and remaining target DNA substrate. Just before loading on a 20% native gel, 0.5% SDS was added to denature Twinkle. The fluorescence intensities of the target substrate DNA band (which migrated slowly in the gel) and the displaced IncS band (which migrated faster) were quantified to determine the fraction of strand displaced. The fraction of displaced IncS was plotted as a function of time to determine kinetics of TMSD. As observed in the quenched flow results, the spontaneous TMSD reaction did not produce a significant strand displacement of IncS at the end of 1 hour (˜3% at 60 minutes) (FIG. 1D, right side and FIG. 1E). Similar reaction conducted in presence of Twinkle showed ˜95% displacement of IncS at 60 minutes, showing that Twinkle catalyzed the TMSD reactions on long IncS-TS duplexes (FIG. 1D, left side and FIG. 1E). The initial rate of strand displacement in presence of Twinkle was 0.45 fraction·min⁻¹ in comparison to 0.002 fraction·min⁻¹ rate of spontaneous TMSD reactions (FIG. 1F).

TMSD reactions with Twinkle concentrations ranging from 10 nM to 80 nM (hexameric concentrations), while keeping the concentrations of target DNA substrate and IS at 10 nM and 40 nM, respectively were performed (FIG. 1G). The rate of TMSD reactions as determined by stopped-flow fluorescence assay increased with Twinkle concentration in a sigmoidal fashion, providing a midpoint at around ˜31 nM Twinkle hexamer (FIG. 1H). These results demonstrate that Twinkle forms a catalytically active complex with the DNA substrate and requires a minimum critical concentration to catalyze these reactions. For additional experiments, a 40 nM Twinkle concentration was employed. Interestingly, in the assays performed, no spontaneous TMSD reactions were observed in absence of Twinkle. One way to increase the likelihood of spontaneous reactions is to increase the concentration of IS, as the rate of TMSD reactions is sensitive to the IS concentration. TMSD reactions were performed with IS concentrations increasing from 40 nM to 1.4 μM and measured the rates of these reactions using stopped-flow fluorescence assay (FIG. 1G). The highest IS concentration used did not produce a spontaneous TMSD reactions. Moreover, the twinkle catalyzed TMSD reaction rates at these IS concentrations marginally increased from 1.14 min⁻¹ to 1.62 min⁻¹ (FIG. 1I).

The surprising absence of spontaneous TMSD reaction under the conditions used can be explained by the long DNA length to be displaced. Most of the reported TMSD reactions are performed using DNA substrates comprising 5 to 20 nucleotides. Our 34 nucleotide IncS-TS duplex hinders the ability of IS to completely displace the IncS within the used time-frame. To confirm this possibility and to validate the stopped flow assay, a different set of DNA substrates with a shorter region to be displaced (15 nucleotides) and a 7 nucleotide long toehold were designed (FIG. 1J). Oligonucleotide sequences from a previously published study (3) (see Table 1, Oligo ID: Short oligos) with a minor modification were employed. For example, A was replaced with G at the 3′ end of TS and three dGs added at the 3′ end of TS, thereby forming a three-nucleotide overhang to quench the fluorescence from the FAM moiety attached to the annealed IncS. The modified substrates provided the rates of spontaneous TMSD reactions to be 1.92 min⁻¹ (FIGS. 1K and 1L). The TMSD reaction rate increased by nearly five folds to 9.6 min⁻¹ when Twinkle was added to the reactions, showing the ability of Twinkle to catalyze reactions performed with shorter, more permissive substrates (FIGS. 1K and 1L). These findings demonstrate that Twinkle can be used to achieve faster rates of TMSD even with the substrates which support fast rates of spontaneous TMSD.

### Strand-Displacement Activity of Twinkle Requires Toehold Formation

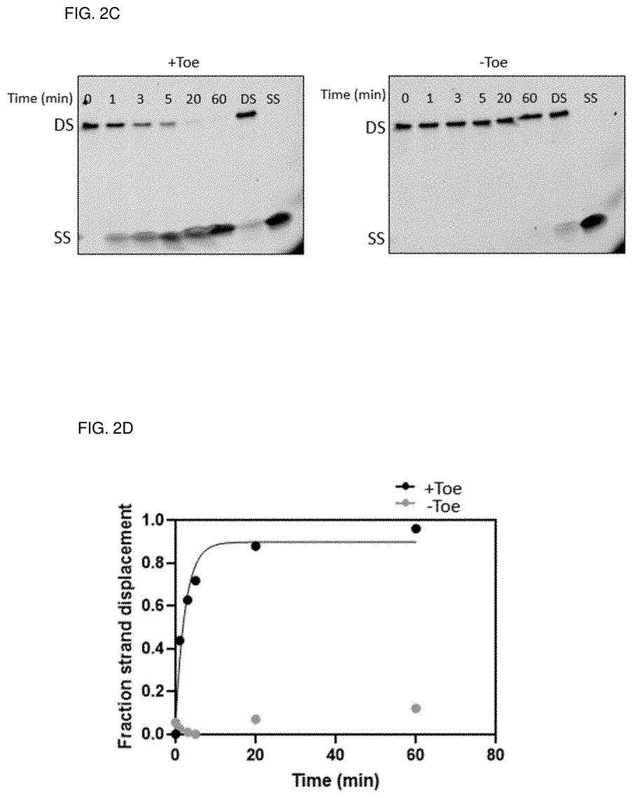

Twinkle can catalyze recombination-mediated exchange of homologous DNA strands in a nucleotide hydrolysis dependent reaction. To confirm that the strand displacement reaction observed in presence of Twinkle was truly toehold mediated, and not the result of Twinkle catalyzing a toehold-independent recombination reaction, the requirement of the ‘toe’ in these Twinkle catalyzed strand displacement reactions was tested with an IS that was missing the six-nucleotide long toe region complementary to the toehold in the TS (FIG. 2A). The real-time stopped-flow assay to monitor TMSD was conducted, and the results were confirmed using gel-based assay. Interestingly, we did not see displacement of IncS DNA in reactions conducted with ‘toe-less’ IS (FIGS. 2B-D). This experiment clearly demonstrated that Twinkle, unlike a true recombinase, merely catalyzes the TMSD reaction and does not facilitate a recombination reaction in absence of toehold formation. As discussed below, the presence of magnesium and ATP does not alter this property and Twinkle requires the ‘toe’ to catalyze the strand displacement reaction.

### Controlling the Kinetics of Twinkle Catalyzed TMSD Reactions

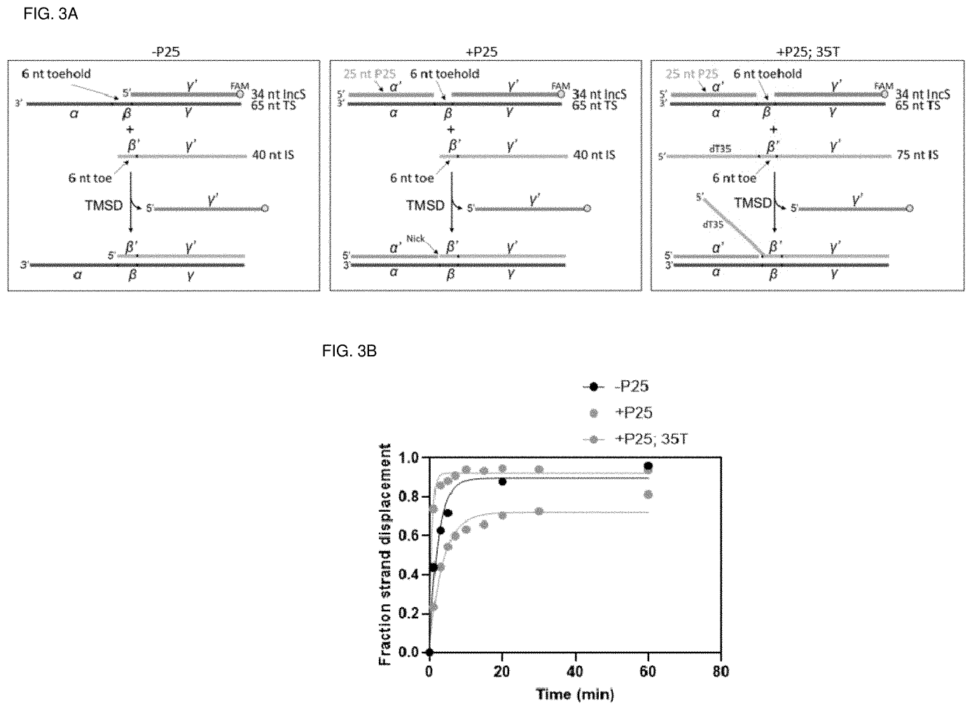

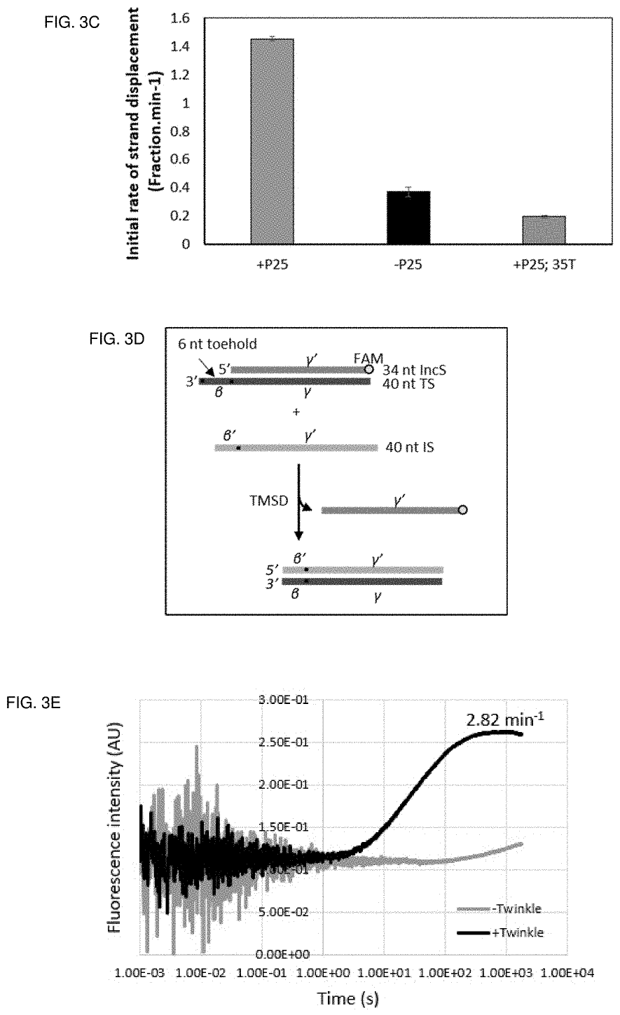

The presence of Twinkle increased the rate of TMSD for short DNA substrates and made the TMSD reactions feasible for the longer ones. Whether the kinetics of these reactions can be controlled by making changes to the substrate design was further assessed. In the original long substrate design, a 31 nucleotide long 3′ ssDNA TS overhang was employed wherein the TS is annealed to IncS (FIG. 3A, Left panel). This overhang was used as a handle to fine tune the kinetics of Twinkle catalyzed TMSD reactions. A 25 nucleotide long complementary oligo (P25) was annealed to the 3′ ssDNA overhang of the TS. The annealing of P25 creates a nick between the 3′ end of P25 and 5′ end of the IS during docking of the IS on toehold (FIG. 3A, Middle panel). The presence of the P25 oligo increased the rate of TMSD over three folds in comparison to the TMSD reaction rates in absence of the annealed oligo (FIGS. 3B and 3C). The increase in TMSD rates with the target substrate DNA with annealed P25 appears to be due to stabilization of docking intermediate by stacking interactions between the nitrogenous bases of P25 and IS.

Changes introduced to the IS length can also be used to tune the kinetics of Twinkle catalyzed TMSD. For example, the length of the IS was increased by adding a 35 nucleotide dT tail to the 5′ end of the original IS, while also using the target DNA substrate annealed to P25 (FIG. 3A, Right panel). The presence of 35 nucleotide 5′ tail in the IS reduced the rate of Twinkle catalyzed TMSD by more than 8-fold in comparison to reactions with IS with no 5′ tail (FIGS. 3B and 3C). In addition to the above modifications, removal of 3′ overhang of the TS increased the rate of Twinkle catalyzed TMSD by over two-fold (FIGS. 3D and 3E).

Twinkle does not Require its Helicase Activity to Catalyze TMSD

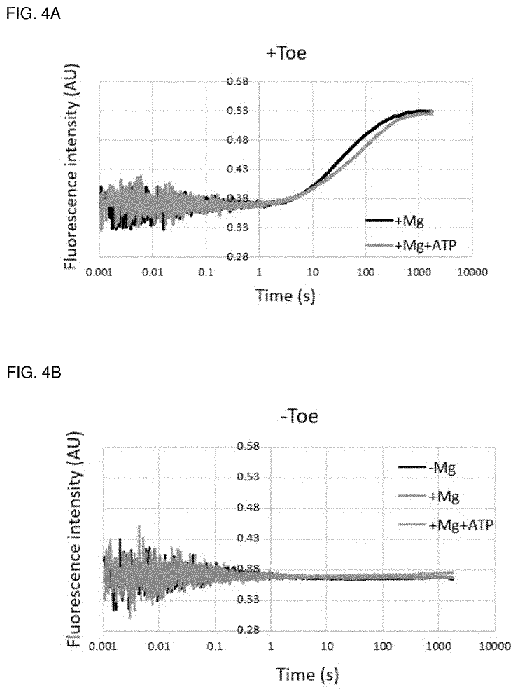

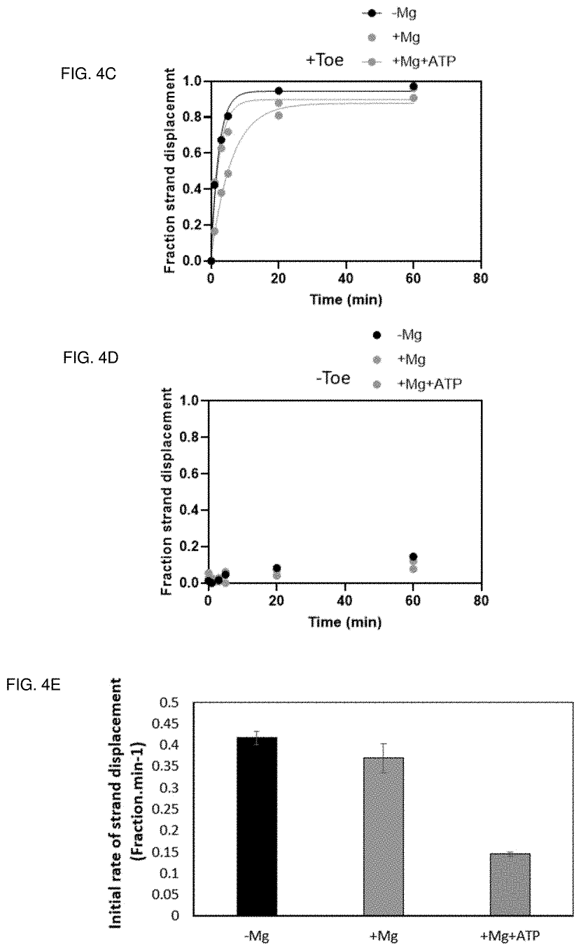

We have shown previously that Twinkle has annealing activity and can catalyze the annealing of two complementary DNA strands in absence of nucleotide (NTP) hydrolysis. In contrast, Twinkle's unwinding activity as well as its strand exchange activity requires the presence of NTP and Mg⁺², or in other words, requires NTPase driven translocation of Twinkle on the DNA. The effect of Twinkle translocation on its catalysis of TMSD reactions and whether Twinkle utilizes ATP hydrolysis to translocate over the IS-TS DNA duplex to actively facilitate branch migration step, further increasing the kinetics of TMSD reactions, was determined. The assay was designed having conditions conducive for Twinkle translocation via inclusion of Mg⁺² and ATP (a preferred fuel for Twinkle translocation) in the reaction mixture. Each of the previous TMSD reactions reported above were conducted in absence of ATP, demonstrating that Twinkle can catalyze these reactions without requiring its directional translocation over the DNA substrates. We conducted TMSD reactions with Twinkle and 10 mM magnesium acetate in presence and absence of 2 mM ATP. Presence of Mg⁺² and ATP did not increase the rate of Twinkle catalyzed TMSD reactions suggesting that Twinkle translocation does not actively support strand displacement under these conditions (FIG. 4A). Instead, the presence of Mg⁺² decreased the rates slightly from 1.2 min⁻¹ to 1.14 min⁻¹, and the combination of Mg⁺² and ATP further reduced the rates to 0.78 min⁻¹ (FIG. 4A)

These findings were further confirmed with the gel-based assay. Similar to the results obtained from the stopped-flow experiments, addition of 10 mM magnesium acetate slightly reduced the rates of Twinkle catalyzed TMSD (FIGS. 4C and 4E). Addition of ATP further decreased the rate to 0.144 fraction·min⁻¹. It is known that Mg⁺² and ATP can change Twinkle's oligomeric state. Reduced rates of Twinkle catalyzed TMSD could possibly be a result of alteration of Twinkle's oligomeric structure in presence of Mg⁺² and ATP. Controlled reactions lacking Twinkle did not produce any displaced DNA product within the timeframe of reaction. We have reported earlier that Twinkle can catalyze exchange of homologous DNA strands in presence of Mg⁺² and ATP. We wanted to explore if the presence of ‘toe’ in IS is essential in Twinkle catalyzed strand displacement reactions containing magnesium and ATP. Strand displacement reactions were conducted with the IS lacking the six nucleotide ‘toe’. No strand displacement was observed even in the presence of Mg⁺² and ATP, showing that Twinkle necessarily requires toehold formation for its catalysis of these reactions and its translocation on the DNA substrates does not change this indispensable requirement (FIGS. 4B and 4D).

### Rates of Twinkle Catalyzed TMSD are Independent of the Direction of Branch Migration

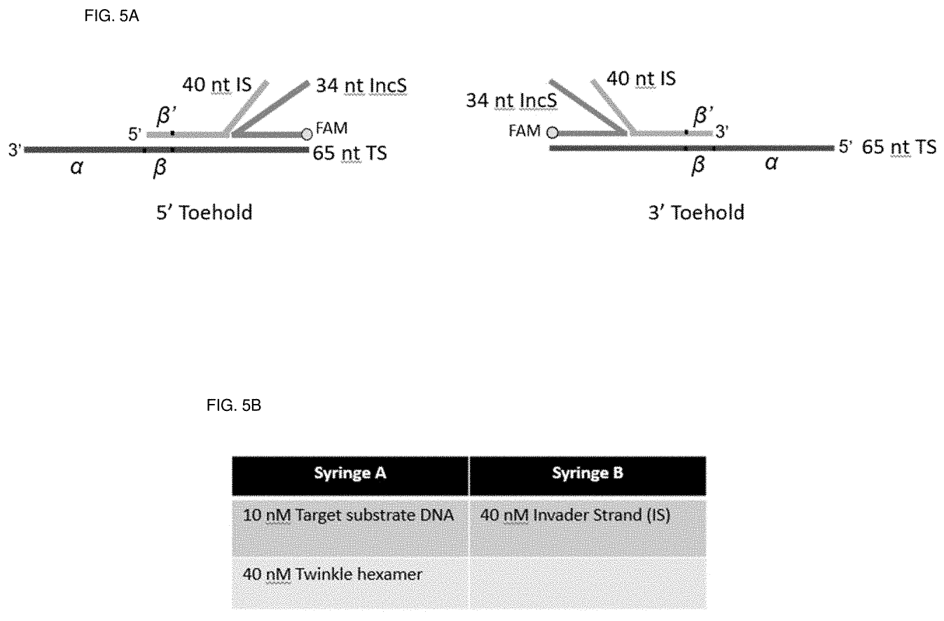

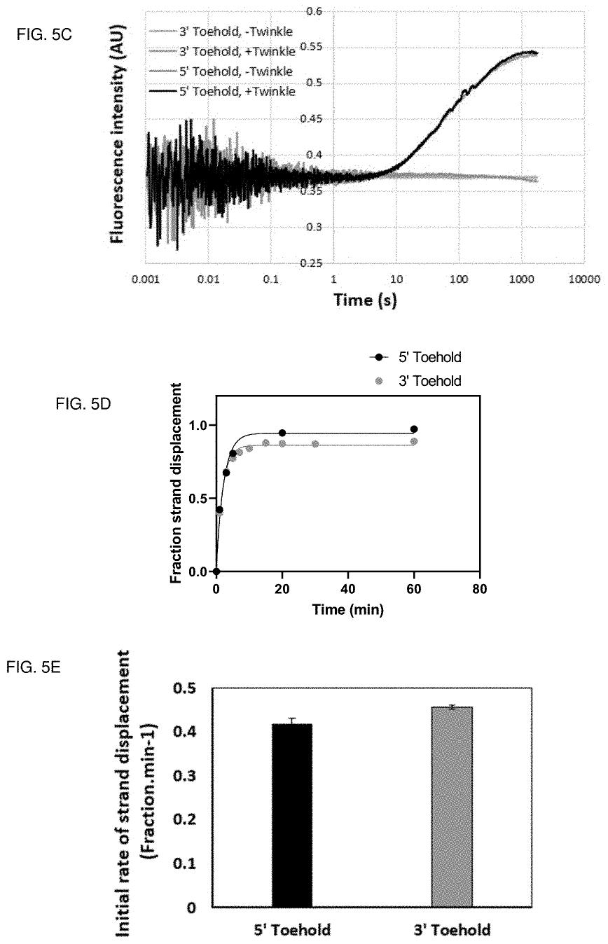

Like other RecA-type hexameric helicases, Twinkle utilizes NTP hydrolysis to translocate unidirectionally on ssDNA in 5′ to 3′ direction. Twinkle may preferentially bind with its N-terminal tier towards 5′ end of ssDNA and its C-terminal tier towards 3′ end to achieve its directionality of translocation. We reasoned that such preferential binding would affect the TMSD reactions catalyzed by Twinkle, limiting its applicability in DNA nanotechnology. The target DNA substrates (IncS:TS) that we used for this study result in the displacement of the IncS from its 5′ end with the docking taking place at the 5′ end of the IS. Owing to its preferential binding orientation to the target DNA, it is possible that the ability of Twinkle to catalyze TMSD is compromised in reactions where toehold docking takes place on the 3′ end of the IS rather than at the 5′ end. To test this possibility, we designed a modified set of TS, IncS and IS with exactly same nucleotide sequence but with opposite polarity. Thus, in this case while the sequence of nucleotide base-pairs formed and displaced during TMSD remains same, the 3′ end of the IS participates in the toehold docking and the branch migration takes place in 3′ to 5′ direction (FIG. 5A).

We performed TMSD reactions using stopped-flow assay in absence of Twinkle which, as expected, did not show a discernible change in fluorescence signal within 30 minutes of reaction time (FIGS. 5B and 5C). The presence of Twinkle catalyzed the TMSD reactions conducted with the target DNA substrate and IS with reversed polarity (FIG. 5C). Twinkle's ability to catalyze TMSD reactions did not depend on the direction of branch migration, and it accelerated the rate of TMSD to similar extent (with rate of 1.32 min⁻¹). We also confirmed these results in a gel-based experiment. From our gel-based assay, the rate of Twinkle catalyzed TMSD reaction with reversed polarity substrates was 0.468 fraction·min⁻¹, which was similar to the rates measured for the original substrate design (0.416 fraction·min⁻¹) (FIGS. 5D and 5E).

### Twinkle Catalyzed TMSD is Sensitive to Nucleotide Mismatches

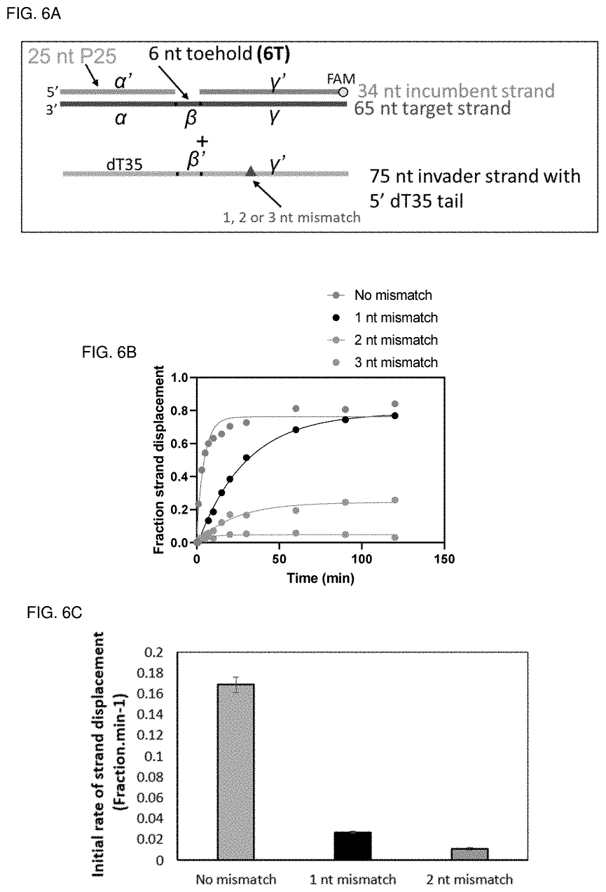

One of the major biotechnological applications of TMSD reactions is the detection of single nucleotide polymorphisms (SNPs). TMSD reactions rates are sensitive to the nucleotide mismatches between the TS and IS. The rates of such TMSD reactions are lower than those conducted with correctly base-paired IS and TS and this kinetic difference can be used to detect a specific SNP within an array of sequences. This differential kinetics can also be utilized for developing diagnostic assays to detect different variants of pathogenic viruses. It has been shown that the extent of reduction in rate depends on the number of mismatches as well as the position of the nucleotide mismatch from the toehold. We wanted to know if this property of TMSD reactions is retained in the reactions catalyzed by Twinkle. To explore this, we designed ISs resulting in 1, 2 or 3 contiguous nucleotide mismatches when they are annealed to the TS (FIG. 6A). The ISs with mismatches resulted in slower TMSD rates, confirming that TMSD reactions catalyzed by Twinkle can indeed be used to develop applications that rely on this kinetic property (FIGS. 6B and 6C). The TMSD reaction rates decreased with increasing number of the mismatches between IS and TS. The initial rates determined from the gel-based assay for substrates resulting in 0, 1 or 2 mismatches were 0.17, 0.03 and 0.003 fraction·min⁻¹ respectively (FIG. 6C). The TMSD for the DNA substrate with 3 nucleotide mismatch was very slow and the rate could not be determined accurately.

### Example II

### A Truncated Twinkle C-Terminal Domain Fragment Efficiently Catalyzes TMSD Reactions

Highly specific molecular interactions of DNA sequences, along with the high programmability of strand-displacement reactions for achieving complex cascades and kinetic predictability of TMSD reactions make DNA strand displacement reactions an attractive tool for use in molecular computing (Garg et al., 2018, Seelig et al., 2006, Zhou et al., 2016). The potential of TMSD reactions in development of DNA logic circuits has been realized with successful implementation of digital logic circuits such as AND, OR, XOR, NOR and NAND gates (Seelig et al., 2006, Zhou et al., 2016). Strategies have developed to achieve combinations of these gates, giving rise to complex digital functions (Qian and Winfree, 2011). The applications of these computing functions have been explored in biosensing (Arter et al., 2020), controlling cellular functions (Qu et al., 2017) and controlling the drug pharmacokinetics (Xiao et al., 2019). Although, the TMSD driven molecular computing has shown great potential, it has some major limitations which require further development of this technology. The requirement of multilayered, complex cascades necessary to create complex logic functions limits the expansion of computing range possible with DNA strand displacement reactions. Moreover, as the number of layers is increased to accomplish higher complexity, the computation time also increases, making DNA based computation slow and impractical for broader application. Use of some restriction enzymes (Zhang et al., 2020) and deoxyribozymes (Zheng et al., 2019) have been successfully explored to overcome these limitations to a certain extent, showing that the enzyme driven TMSD might be a way forward.

Twinkle is a ring-forming hexameric DNA helicase which localizes to mitochondrial nucleoids and is involved in adenosine triphosphate (ATP)-dependent unwinding of double-stranded DNA (dsDNA). The linker helix forms a stable helix bundle at the surface of the helicase domain of the neighboring subunit, causing the N-terminal domain to rest on top of the neighboring helicase domain. The binding of nucleoside triphosphates (NTPs) occurs at the subunit interface and, upon NTP hydrolysis, the helicase domains rotate and shift in relation to one another to provide the mechanical force required for DNA unwinding. The linker region of T7 gp4 is crucial for both oligomerization and helicase activity.

### Twinkle C-Terminal Domain Catalyzes TMSD Reactions

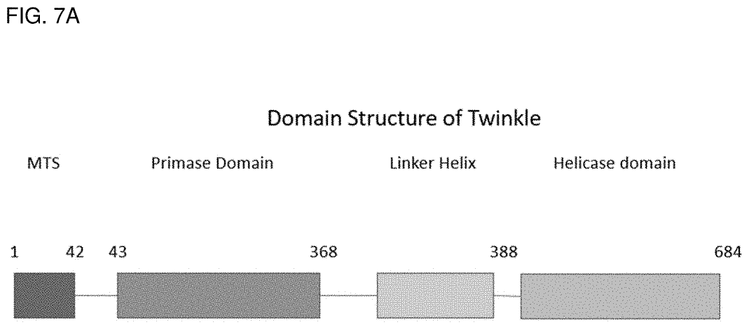

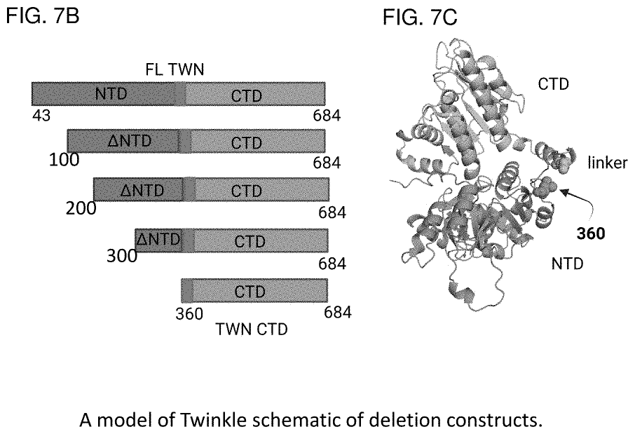

In this study, we performed a TMSD assay using a truncated version of Twinkle As noted above, Twinkle consists of a C-terminal domain (CTD) and an N-terminal domain (NTD) connected by a flexible linker. Like other of RecA-type hexameric helicases, Twinkle hexamer's C-terminal tier has NTPase active sites and thus is preferable for Twinkle's translocation and unwinding activities. Unlike its ancestral homologue, bacteriophage T7 gp4, Twinkle's NTD does not possess primase activity and its role in mitochondrial DNA replication is not clearly understood. In order to further elucidate the mechanism of the role Twinkle domains play in strand-displacement activity, a deletion construct of Twinkle with the CTD and a part of the linker was expressed in E. coli. The purified CTD was further tested for its ability to catalyze TMSD reactions. For comparison, a tag-less version of full-length Twinkle was also expressed in and purified from E. coli. We performed TMSD reactions with the full-length Twinkle or with the purified CTD. Interestingly, the Twinkle CTD was similarly active in catalyzing TMSD reactions performed with our substrates, demonstrating that it is the Twinkle CTD that confers Twinkle with its strand-displacement activity (FIGS. 7B-7D).

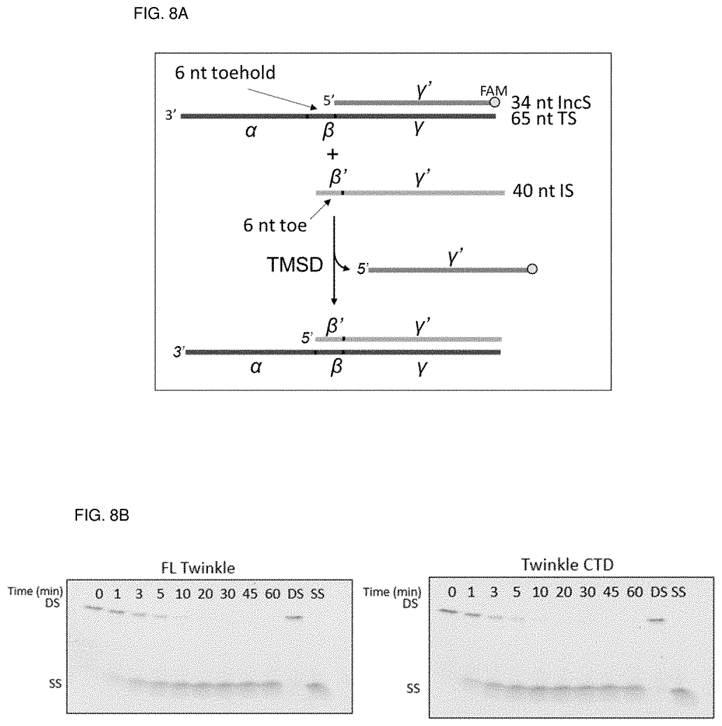

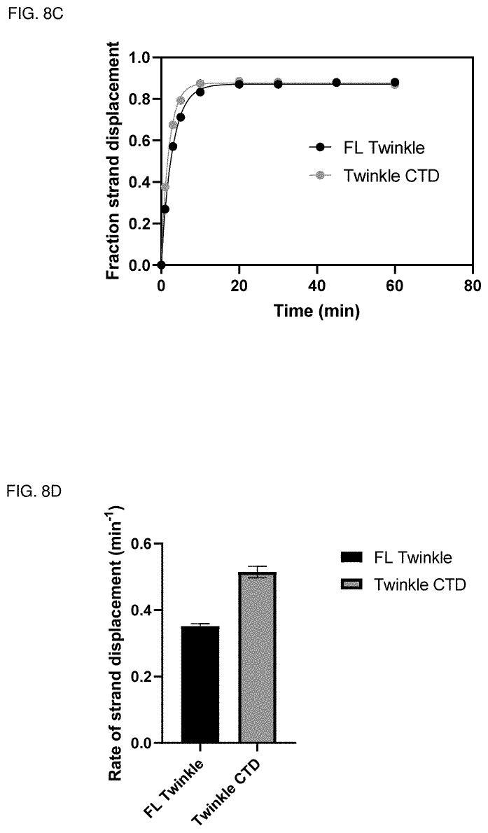

Twinkle CTD can be expressed as a fusion protein with yeast SUMO protein (Small Ubiquitin-like Modifier) covalently attached to the N-terminal end of Twinkle CTD (TWN-CTD with SUMO). See FIG. 8. The addition of the SUMO tag to the CTD increased the solubility and increased the efficiency of purification relative to the wild-type full length protein. Additional variants can be generated with commonly used affinity tags, epitope tags, and fluorescent molecules/proteins. See for example Costa et al. Front. Microbiol. (2014) (doi.org/10.3389/fmicb.2014.00063). New variants can be prepared by attachment to solid supports such as microbeads, resins, or immobilized in gels such as calcium alginate.

The Twinkle CTD sequence can be modified to a non-naturally occurring sequence through systematic bioinformatics and mutagenesis methods. Twinkle is found in the mitochondria of all metazoans and many non-metazoan eukaryotes. The sequence differences between them can be used to engineer the Twinkle CTD variant.

Sequences useful for the practice of the present invention.

| Amino Acid Sequences | |
|---|---|
| Human Mitochondrial Helicase, | MGSSHHHHHHGSGLVPRGSASMSDSEVNQEAKPEVKPEVKPE |
| Twinkle, C-terminal Domain | THIN |
| Construct (TWN-CTD) with SUMO | LKVSDGSSEIFFKIKKTTPLRRLMEAFAKRQGKEMDSLRFLYDG |
| tag amino acid Sequence, as | IRIQA |
| expressed (SEQ ID NO: 19) | 360 |
| | 382 |
| | DQTPEDLDMEDNDIIEAHREQIGGS |
| | LPAWHKSIVSFRQLREEVLGELS |
| | 383 |
| | 430 |
| | NVEQAAGLRWSRFPDLNRILKGHRKGELTVFTGPTGSG |
| | KTTFISEYAL |
| | 431 |
| | 475 |
| | DLCSQGVNTLWGSFEISNVRLARVMLTQFAEGRLEDQL |
| | DKYDHWA |
| | 476 |
| | 522 |
| | DRFEDLPLYFMTFHGQQSIRTVIDTMQHAVYVYDICHVII |
| | DNLQFMM |
| | 523 |
| | 570 |
| | GHEQLSTDRIAAQDYIIGVFRKFATDNNCHVTLVIHPRKE |
| | DDDKELQT |
| | 571 |
| | 617 |
| | ASIFGSAKASQEADNVLILQDRKLVTGPGKRYLQVSKNR |
| | FDGDVGVF |
| | 618 |
| | 666 |
| | PLEFNKNSLTFSIPPKNKARLKKIKDDTGPVAKKPSSGKK |
| | GATTONSEI |
| | 667 684 |
| | CSGQAPTPDQPDTSKRSK |
| Human Mitochondrial Helicase, | 360 |
| Twinkle, C-terminal Domain | 406 |
| Construct (TWN-CTD) with SUMO | SLPAWHKSIVSFRQLREEVLGELSNVEQAAGLRWSRFPD |
| tag amino acid Sequence, as used in | LNRILKGHR |
| assays (SUMO tag removed) (SEQ | 407 |
| ID NO: 18) | 454 |
| | KGELTVFTGPTGSGKTTFISEYALDLCSQGVNTLWGSFEI |
| | SNVRLARV |
| | 455 |
| | 500 |
| | MLTQFAEGRLEDQLDKYDHWADRFEDLPLYFMTFHGQ |
| | QSIRTVIDT |
| | 501 |
| | 547 |
| | MQHAVYVYDICHVIIDNLQFMMGHEQLSTDRIAAQDYII |
| | GVFRKFAT |
| | 548 |
| | 595 |
| | DNNCHVTLVIHPRKEDDDKELQTASIFGSAKASQEADNV |
| | LILQDRKLV |
| | 596 |
| | 643 |
| | TGPGKRYLQVSKNRFDGDVGVFPLEFNKNSLTFSIPPKN |
| | KARLKKIKD |
| | 644 684 |
| | DTGPVAKKPSSGKKGATTQNSEICSGQAPTPDQPDTSKR |
| | SK |

Legend

SUMO tag from yeast in italics (SEQ ID NO: 17) Amino acids 360-684 from canonical Human Twinkle protein in bold (SEQ ID NO: 18)
a single amino acid remains (Serine) after cleavage of the SUMO tag on the N-terminus of the TWN-CTD
TWN-CTD sequence sourced from human
SUMO tag sequence sourced from yeast

| DNA Sequences | |
|---|---|
| Human Mitochondrial | TGGCGAATGGGACGCGCCCT |
| Helicase, Twinkle, | GTAGCGGCGCATTAAGCGCG |
| C-terminal Domain | GCGGGTGTGGTGGTTACGCG |
| Construct (TWN-CTD) | CAGCGTGACCGCTACACTTG |
| inserted in frame | CCAGCGCCCTAGCGCCCGCT |
| with pET 28 Vector | CCTTTCGCTTTCTTCCCTTC |
| SUMO tag | CTTTCTCGCCACGTTCGCCG |
| DNA Sequence | GCTTTCCCCGTCAAGCTCTA |
| (SEQ ID NO: 20) | AATCGGGGGCTCCCTTTAGG |
| | GTTCCGATTTAGTGCTTTAC |
| | GGCACCTCGACCCCAAAAAA |
| | CTTGATTAGGGTGATGGTTC |
| | ACGTAGTGGGCCATCGCCCT |
| | GATAGACGGTTTTTCGCCCT |
| | TTGACGTTGGAGTCCACGTT |
| | CTTTAATAGTGGACTCTTGT |
| | TCCAAACTGGAACAACACTC |
| | AACCCTATCTCGGTCTATTC |
| | TTTTGATTTATAAGGGATTT |
| | TGCCGATTTCGGCCTATTGG |
| | TTAAAAAATGAGCTGATTTA |
| | ACAAAAATTTAACGCGAATT |
| | TTAACAAAATATTAACGTTT |
| | ACAATTTCAGGTGGCACTTT |
| | TCGGGGAAATGTGCGCGGAA |
| | CCCCTATTTGTTTATTTTTC |
| | TAAATACATTCAAATATGTA |
| | TCCGCTCATGAATTAATTCT |
| | TAGAAAAACTCATCGAGCAT |
| | CAAATGAAACTGCAATTTAT |
| | TCATATCAGGATTATCAATA |
| | CCATATTTTTGAAAAAGCCG |
| | TTTCTGTAATGAAGGAGAAA |
| | ACTCACCGAGGCAGTTCCAT |
| | AGGATGGCAAGATCCTGGTA |
| | TCGGTCTGCGATTCCGACTC |
| | GTCCAACATCAATACAACCT |
| | ATTAATTTCCCCTCGTCAAA |
| | AATAAGGTTATCAAGTGAGA |
| | AATCACCATGAGTGACGACT |
| | GAATCCGGTGAGAATGGCAA |
| | AAGTTTATGCATTTCTTTCC |
| | AGACTTGTTCAACAGGCCAG |
| | CCATTACGCTCGTCATCAAA |
| | ATCACTCGCATCAACCAAAC |
| | CGTTATTCATTCGTGATTGC |
| | GCCTGAGCGAGACGAAATAC |
| | GCGATCGCTGTTAAAAGGAC |
| | AATTACAAACAGGAATCGAA |
| | TGCAACCGGCGCAGGAACAC |
| | TGCCAGCGCATCAACAATAT |
| | TTTCACCTGAATCAGGATAT |
| | TCTTCTAATACCTGGAATGC |
| | TGTTTTCCCGGGGATCGCAG |
| | TGGTGAGTAACCATGCATCA |
| | TCAGGAGTACGGATAAAATG |
| | CTTGATGGTCGGAAGAGGCA |
| | TAAATTCCGTCAGCCAGTTT |
| | AGTCTGACCATCTCATCTGT |
| | AACATCATTGGCAACGCTAC |
| | CTTTGCCATGTTTCAGAAAC |
| | AACTCTGGCGCATCGGGCTT |
| | CCCATACAATCGATAGATTG |
| | TCGCACCTGATTGCCCGACA |
| | TTATCGCGAGCCCATTTATA |
| | CCCATATAAATCAGCATCCA |
| | TGTTGGAATTTAATCGCGGC |
| | CTAGAGCAAGACGTTTCCCG |
| | TTGAATATGGCTCATAACAC |
| | CCCTTGTATTACTGTTTATG |
| | TAAGCAGACAGTTTTATTGT |
| | TCATGACCAAAATCCCTTAA |
| | CGTGAGTTTTCGTTCCACTG |
| | AGCGTCAGACCCCGTAGAAA |
| | AGATCAAAGGATCTTCTTGA |
| | GATCCTTTTTTTCTGCGCGT |
| | AATCTGCTGCTTGCAAACAA |
| | AAAAACCACCGCTACCAGCG |
| | GTGGTTTGTTTGCCGGATCA |
| | AGAGCTACCAACTCTTTTTC |
| | CGAAGGTAACTGGCTTCAGC |
| | AGAGCGCAGATACCAAATAC |
| | TGTCCTTCTAGTGTAGCCGT |
| | AGTTAGGCCACCACTTCAAG |
| | AACTCTGTAGCACCGCCTAC |
| | ATACCTCGCTCTGCTAATCC |
| | TGTTACCAGTGGCTGCTGCC |
| | AGTGGCGATAAGTCGTGTCT |
| | TACCGGGTTGGACTCAAGAC |
| | GATAGTTACCGGATAAGGCG |
| | CAGCGGTCGGGCTGAACGGG |
| | GGGTTCGTGCACACAGCCCA |
| | GCTTGGAGCGAACGACCTAC |
| | ACCGAACTGAGATACCTACA |
| | GCGTGAGCTATGAGAAAGCG |
| | CCACGCTTCCCGAAGGGAGA |
| | AAGGCGGACAGGTATCCGGT |
| | AAGCGGCAGGGTCGGAACAG |
| | GAGAGCGCACGAGGGAGCTT |
| | CCAGGGGGAAACGCCTGGTA |
| | TCTTTATAGTCCTGTCGGGT |
| | TTCGCCACCTCTGACTTGAG |
| | CGTCGATTTTTGTGATGCTC |
| | GTCAGGGGGGCGGAGCCTAT |
| | GGAAAAACGCCAGCAACGCG |
| | GCCTTTTTACGGTTCCTGGC |
| | CTTTTGCTGGCCTTTTGCTC |
| | ACATGTTCTTTCCTGCGTTA |
| | TCCCCTGATTCTGTGGATAA |
| | CCGTATTACCGCCTTTGAGT |
| | GAGCTGATACCGCTCGCCGC |
| | AGCCGAACGACCGAGCGCAG |
| | CGAGTCAGTGAGCGAGGAAG |
| | CGGAAGAGCGCCTGATGCGG |
| | TATTTTCTCCTTACGCATCT |
| | GTGCGGTATTTCACACCGCA |
| | TATATGGTGCACTCTCAGTA |
| | CAATCTGCTCTGATGCCGCA |
| | TAGTTAAGCCAGTATACACT |
| | CCGCTATCGCTACGTGACTG |
| | GGTCATGGCTGCGCCCCGAC |
| | ACCCGCCAACACCCGCTGAC |
| | GCGCCCTGACGGGCTTGTCT |
| | GCTCCCGGCATCCGCTTACA |
| | GACAAGCTGTGACCGTCTCC |
| | GGGAGCTGCATGTGTCAGAG |
| | GTTTTCACCGTCATCACCGA |
| | AACGCGCGAGGCAGCTGCGG |
| | TAAAGCTCATCAGCGTGGTC |
| | GTGAAGCGATTCACAGATGT |
| | CTGCCTGTTCATCCGCGTCC |
| | AGCTCGTTGAGTTTCTCCAG |
| | AAGCGTTAATGTCTGGCTTC |
| | TGATAAAGCGGGCCATGTTA |
| | AGGGCGGTTTTTTCCTGTTT |
| | GGTCACTGATGCCTCCGTGT |
| | AAGGGGGATTTCTGTTCATG |
| | GGGGTAATGATACCGATGAA |
| | ACGAGAGAGGATGCTCACGA |
| | TACGGGTTACTGATGATGAA |
| | CATGCCCGGTTACTGGAACG |
| | TTGTGAGGGTAAACAACTGG |
| | CGGTATGGATGCGGCGGGAC |
| | CAGAGAAAAATCACTCAGGG |
| | TCAATGCCAGCGCTTCGTTA |
| | ATACAGATGTAGGTGTTCCA |
| | CAGGGTAGCCAGCAGCATCC |
| | TGCGATGCAGATCCGGAACA |
| | TAATGGTGCAGGGCGCTGAC |
| | TTCCGCGTTTCCAGACTTTA |
| | CGAAACACGGAAACCGAAGA |
| | CCATTCATGTTGTTGCTCAG |
| | GTCGCAGACGTTTTGCAGCA |
| | GCAGTCGCTTCACGTTCGCT |
| | CGCGTATCGGTGATTCATTC |
| | TGCTAACCAGTAAGGCAACC |
| | CCGCCAGCCTAGCCGGGTCC |
| | TCAACGACAGGAGCACGATC |
| | ATGCGCACCCGTGGGGCCGC |
| | CATGCCGGCGATAATGGCCT |
| | GCTTCTCGCCGAAACGTTTG |
| | GTGGCGGGACCAGTGACGAA |
| | GGCTTGAGCGAGGGCGTGCA |
| | AGATTCCGAATACCGCAAGC |
| | GACAGGCCGATCATCGTCGC |
| | GCTCCAGCGAAAGCGGTCCT |
| | CGCCGAAAATGACCCAGAGC |
| | GCTGCCGGCACCTGTCCTAC |
| | GAGTTGCATGATAAAGAAGA |
| | CAGTCATAAGTGCGGCGACG |
| | ATAGTCATGCCCCGCGCCCA |
| | CCGGAAGGAGCTGACTGGGT |
| | TGAAGGCTCTCAAGGGCATC |
| | GGTCGAGATCCCGGTGCCTA |
| | ATGAGTGAGCTAACTTACAT |
| | TAATTGCGTTGCGCTCACTG |
| | CCCGCTTTCCAGTCGGGAAA |
| | CCTGTCGTGCCAGCTGCATT |
| | AATGAATCGGCCAACGCGCG |
| | GGGAGAGGCGGTTTGCGTAT |
| | TGGGCGCCAGGGTGGTTTTT |
| | CTTTTCACCAGTGAGACGGG |
| | CAACAGCTGATTGCCCTTCA |
| | CCGCCTGGCCCTGAGAGAGT |
| | TGCAGCAAGCGGTCCACGCT |
| | GGTTTGCCCCAGCAGGCGAA |
| | AATCCTGTTTGATGGTGGTT |
| | AACGGCGGGATATAACATGA |
| | GCTGTCTTCGGTATCGTCGT |
| | ATCCCACTACCGAGATATCC |
| | GCACCAACGCGCAGCCCGGA |
| | CTCGGTAATGGCGCGCATTG |
| | CGCCCAGCGCCATCTGATCG |
| | TTGGCAACCAGCATCGCAGT |
| | GGGAACGATGCCCTCATTCA |
| | GCATTTGCATGGTTTGTTGA |
| | AAACCGGACATGGCACTCCA |
| | GTCGCCTTCCCGTTCCGCTA |
| | TCGGCTGAATTTGATTGCGA |
| | GTGAGATATTTATGCCAGCC |
| | AGCCAGACGCAGACGCGCCG |
| | AGACAGAACTTAATGGGCCC |
| | GCTAACAGCGCGATTTGCTG |
| | GTGACCCAATGCGACCAGAT |
| | GCTCCACGCCCAGTCGCGTA |
| | CCGTCTTCATGGGAGAAAAT |
| | AATACTGTTGATGGGTGTCT |
| | GGTCAGAGACATCAAGAAAT |
| | AACGCCGGAACATTAGTGCA |
| | GGCAGCTTCCACAGCAATGG |
| | CATCCTGGTCATCCAGCGGA |
| | TAGTTAATGATCAGCCCACT |
| | GACGCGTTGCGCGAGAAGAT |
| | TGTGCACCGCCGCTTTACAG |
| | GCTTCGACGCCGCTTCGTTC |
| | TACCATCGACACCACCACGC |
| | TGGCACCCAGTTGATCGGCG |
| | CGAGATTTAATCGCCGCGAC |
| | AATTTGCGACGGCGCGTGCA |
| | GGGCCAGACTGGAGGTGGCA |
| | ACGCCAATCAGCAACGACTG |
| | TTTGCCCGCCAGTTGTTGTG |
| | CCACGCGGTTGGGAATGTAA |
| | TTCAGCTCCGCCATCGCCGC |
| | TTCCACTTTTTCCCGCGTTT |
| | TCGCAGAAACGTGGCTGGCC |
| | TGGTTCACCACGCGGGAAAC |
| | GGTCTGATAAGAGACACCGG |
| | CATACTCTGCGACATCGTAT |
| | AACGTTACTGGTTTCACATT |
| | CACCACCCTGAATTGACTCT |
| | CTTCCGGGCGCTATCATGCC |
| | ATACCGCGAAAGGTTTTGCG |
| | CCATTCGATGGTGTCCGGGA |
| | TCTCGACGCTCTCCCTTATG |
| | CGACTCCTGCATTAGGAAGC |
| | AGCCCAGTAGTAGGTTGAGG |
| | CCGTTGAGCACCGCCGCCGC |
| | AAGGAATGGTGCATGCAAGG |
| | AGATGGCGCCCAACAGTCCC |
| | CCGGCCACGGGGCCTGCCAC |
| | CATACCCACGCCGAAACAAG |
| | CGCTCATGAGCCCGAAGTGG |
| | CGAGCCCGATCTTCCCCATC |
| | GGTGATGTCGGCGATATAGG |
| | CGCCAGCAACCGCACCTGTG |
| | GCGCCGGTGATGCCGGCCAC |
| | GATGCGTCCGGCGTAGAGGA |
| | TCGAGATCTCGATCCCGCGA |
| | AATTAATACGACTCACTATA |
| | GGGGAATTGTGAGCGGATAA |
| | CAATTCCCCTCTAGAAATAA |
| | TTTTGTTTAACTTTAAGAAG |
| | GAGATATACATATGGGCAGC |
| | AGCCATCATCATCATCATCA |
| | CGGCAGCGGCCTGGTGCCGC |
| | GCGGCAGCGCTAGCATGTCG |
| | GACTCAGAAGTCAATCAAGA |
| | AGCTAAGCCAGAGGTCAAGC |
| | CAGAAGTCAAGCCTGAGACT |
| | CACATCAATTTAAAGGTGTC |
| | CGATGGATCTTCAGAGATCT |
| | TCTTCAAGATCAAAAAGACC |
| | ACTCCTTTAAGAAGGCTGAT |
| | GGAAGCGTTCGCTAAAAGAC |
| | AGGGTAAGGAAATGGACTCC |
| | TTAAGATTCTTGTACGACGG |
| | TATTAGAATTCAAGCTGATC |
| | AGACCCCTGAAGATTTGGAC |
| | ATGGAGGATAACGATATTAT |
| | TGAGGCTCACAGAGAACAGA |
| | TTGGTGGATCCctgcctgcc |
| | tggcacaagtccatcgtatc |
| | tttccggcagcttcgggagg |
| | aggtgctaggagaactgtca |
| | aatgtggagcaagcagctgg |
| | cctccgctggagccgctttc |
| | cagacctcaatcgtatcttg |
| | aagggacatcgaaagggcga |
| | gctgacggtcttcacagggc |
| | caacaggcagtggaaagacg |
| | acattcatcagtgagtatgc |
| | cctggatttgtgttcccagg |
| | gggtgaacacactgtggggt |
| | agcttcgagatcagcaatgt |
| | gagactagcccgggtcatgc |
| | tgacacagtttgccgagggg |
| | cggctggaagatcaactgga |
| | caaatatgatcactgggctg |
| | accgctttgaggacctgccc |
| | ctctatttcatgactttcca |
| | tggacagcaaagcatcagga |
| | ctgtaatagatacaatgcaa |
| | catgcagtctacgtctatga |
| | catttgtcatgtgatcatcg |
| | acaacctgcagttcatgatg |
| | ggtcacgagcagctgtccac |
| | agacaggatcgcagctcaag |
| | actacatcatcggggtettt |
| | cggaagtttgcaacagacaa |
| | taactgccatgtgacactgg |
| | tcattcacccccggaaagag |
| | gatgatgacaaggaactgca |
| | gacagcgtccatttttggct |
| | cagccaaagcaagccaggaa |
| | gcagacaatgttctgatcct |
| | gcaggacaggaagctggtaa |
| | ccgggccagggaaacggtat |
| | ctgcaggtgtccaagaaccg |
| | ctttgatggagatgtaggtg |
| | tcttcccgcttgagttcaac |
| | aagaactccctcaccttctc |
| | cattccaccaaagaacaagg |
| | cccggetcaagaagatcaag |
| | gatgacactggaccagtggc |
| | caaaaagccctcttctggca |
| | aaaagggggctacgacacag |
| | aactctgagatttgctcagg |
| | ccaggcccccactcccgacc |
| | agccagacacctccaagegt |
| | tcaaagtaaagaCAAGCTTA |
| | GGTATTTATTCGGCGCAAAG |
| | TGCGTCGGGTGATGCTGCCA |
| | ACTTAGTCGAGCACCACCAC |
| | CACCACCACTGAGATCCGGC |
| | TGCTAACAAAGCCCGAAAGG |
| | AAGCTGAGTTGGCTGCTGCC |
| | ACCGCTGAGCAATAACTAGC |
| | ATAACCCCTTGGGGCCTCTA |
| | AACGGGTCTTGAGGGGTTTT |
| | TTGCTGAAAGGAGGAACTAT |
| | ATCCGGAT |

Legend

Vector backbone SUMO tag in italics (SEQ ID NO: 21); TWN-CTD and termination codon is underlined (SEQ ID NO: 22).

### REFERENCES

1. Sen, D., Nandakumar, D., Tang, G. Q. and Patel, S. S., 2012. Human mitochondrial DNA helicase TWINKLE is both an unwinding and annealing helicase. Journal of Biological Chemistry, 287(18), pp. 14545-14556.
2. Sen, D., Patel, G. and Patel, S. S., 2016. Homologous DNA strand exchange activity of the human mitochondrial DNA helicase TWINKLE. Nucleic acids research, 44(9), pp. 4200-4210.
3. Zhang, D. Y. and Winfree, E., 2009. Control of DNA strand displacement kinetics using toehold exchange. Journal of the American Chemical Society, 131(47), pp. 17303-17314.
4. Simmel, F. C., Yurke, B. and Singh, H. R., 2019. Principles and applications of nucleic acid strand displacement reactions. Chemical reviews, 119(10), pp. 6326-6369.
ARTER, W. E., YUSIM, Y., PETER, Q., TAYLOR, C. G., KLENERMAN, D., KEYSER, U. F. & KNOWLES, T. P. J. 2020. Digital Sensing and Molecular Computation by an Enzyme-Free DNA Circuit. ACS Nano, 14, 5763-5771.
GARG, S., SHAH, S., BUT, H., SONG, T., MOKHTAR, R. & REIF, J. 2018. Renewable Time-Responsive DNA Circuits. Small, e1801470.
QIAN, L. & WINFREE, E. 2011. Scaling up digital circuit computation with DNA strand displacement cascades. Science, 332, 1196-201.
QU, X., WANG, S., GE, Z., WANG, J., YAO, G., LI, J., ZUO, X., SHI, J., SONG, S., WANG, L., LI, L., PEI, H. & FAN, C. 2017. Programming Cell Adhesion for On-Chip Sequential Boolean Logic Functions. J Am Chem Soc, 139, 10176-10179.
SEELIG, G., SOLOVEICHIK, D., ZHANG, D. Y. & WINFREE, E. 2006. Enzyme-free nucleic acid logic circuits. Science, 314, 1585-8.
XIAO, M., LAI, W., WANG, F., LI, L., FAN, C. & PEI, H. 2019. Programming Drug Delivery Kinetics for Active Burst Release with DNA Toehold Switches. J Am Chem Soc, 141, 20354-20364.
ZHANG, X., ZHANG, Q., LIU, Y., WANG, B. & ZHOU, S. 2020. A molecular device: A DNA molecular lock driven by the nicking enzymes. Comput Struct Biotechnol J, 18, 2107-2116.
ZHENG, X., YANG, J., ZHOU, C., ZHANG, C., ZHANG, Q. & WEI, X. 2019. Allosteric DNAzyme-based DNA logic circuit: operations and dynamic analysis. Nucleic Acids Res, 47, 1097-1109.
ZHOU, C., LIU, D., WU, C., LIU, Y. & WANG, E. 2016. Integration of DNA and graphene oxide for the construction of various advanced logic circuits. Nanoscale, 8, 17524-17531.

### Example III

### Kinetics of Twinkle Catalysis of the TMSD Reaction

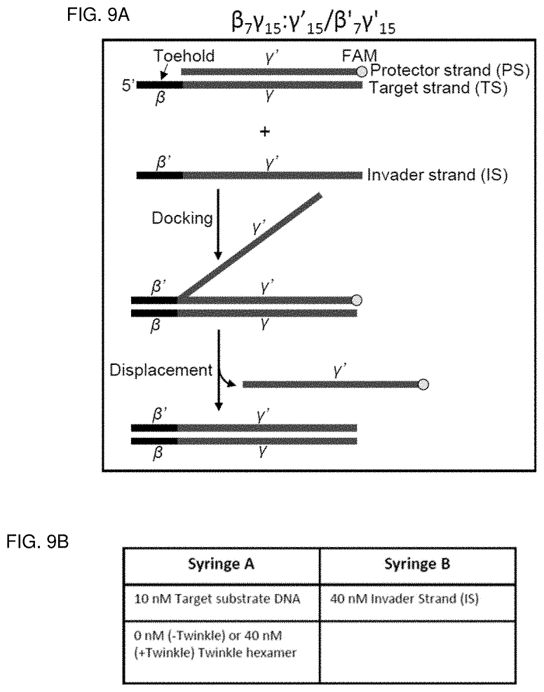

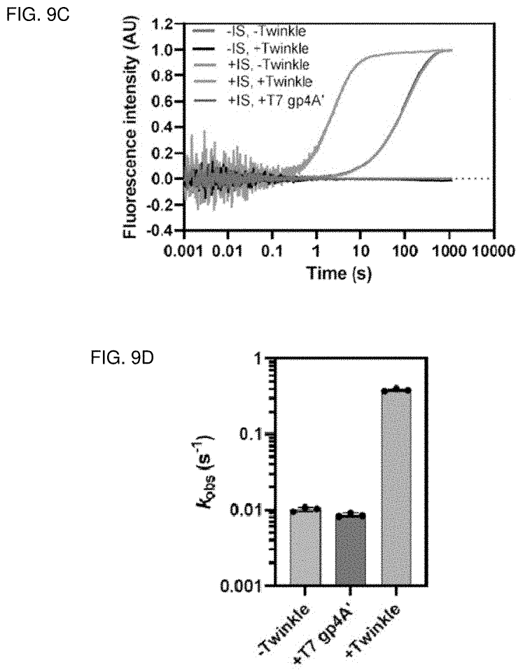

To further assess the specificity and kinetics of the TMSD reaction, we used a 15-bp target duplex DNA created by annealing complementary regions of a target strand (TS, β₇γ₁₅) and a protector strand (PS, γ′₁₅). Adjacent to the duplex region, the target strand contained a 7-nt ‘toehold’ region (β₇), which allowed a homologous invader strand (IS, β′₇γ′₁₅) to dock and invade the duplex DNA (in the displacement domain) and displace the 15-nt PS (γ′₁₅). To monitor the TMSD reaction, the PS was labeled with a fluorescein fluorophore (FAM) at its 5′ end, and the complementary TS was designed to contain a string of four dG residues (one dG residue anneals to the 5′ end of PS participating in the displacement domain while 3 dGs form a 3-nt overhang) to quench the FAM fluorescence (FIG. 9A, Table 1). When the PS is annealed to the TS, FAM fluorescence is low, and when the PS is displaced by the IS, FAM fluorescence increases, providing a real-time assay to measure the TMSD kinetics.

The precise kinetics of the TMSD reaction were measured on a stopped-flow instrument that allows rapid mixing of samples and fluorescence measurement in millisecond time scales. Briefly, one of the stopped-flow syringes was loaded with the TS:PS target duplex (represented as β₇γ₁₅:γ′₁₅, 10 nM) with or without Twinkle (40 nM), and the second syringe was loaded with the IS (β′₇γ′₁₅, 40 nM) (FIG. 9B). The contents of the two syringes were rapidly mixed, and the fluorescence intensity was measured in real-time (FIG. 9C). In the absence of Twinkle, we observed that the TMSD reaction occurred within 10 minutes. Interestingly, when Twinkle was added, the TMSD reaction reached completion within one minute (FIG. 9C).

The kinetic data were fitted to a 1-exponential kinetic model, which provided an average spontaneous TMSD observed rate of 0.01 s⁻¹ and Twinkle-catalyzed TMSD rate of 0.38 s⁻¹ (FIG. 9D). Our results show that Twinkle catalyzes the rate of the TMSD reaction and increases its observed rate by ˜38 folds. Since this is a novel activity of Twinkle, these results were confirmed with several control experiments. The stopped flow traces with no added IS did not show fluorescence increase within the time frame of the reactions, showing that the target dsDNA (β₇γ₁₅:γ′₁₅) does not unwind spontaneously or in presence of Twinkle (FIG. 9C). Furthermore, this activity is unique to Twinkle as homologous bacteriophage T7 helicase-primase gp4A′ did not accelerate the TMSD reaction beyond the spontaneous TMSD rates (FIGS. 9C and 9D).

We investigated whether increased Twinkle concentrations would increase the TMSD rates further. Twinkle concentration was increased from 0 nM to 80 nM, and target dsDNA (β₇γ₁₅:γ′₁₅) and IS (β′₇γ′₁₅) were held constant at 10 nM and 40 nM, respectively (FIG. 9E). Within the range of Twinkle concentration used, the TMSD reaction rates increased in hyperbolic fashion (FIG. 9F), providing a Twinkle K_{M} of 36.87±6.45 nM (FIG. 9G). Next, we measured TMSD kinetics at higher IS concentration of 100 nM while keeping the Twinkle concentration regime (0 to 80 nM) and target dsDNA (10 nM) unchanged. This indicates that the TMSD rate is dependent on IS concentration, and, for each Twinkle concentration used, the observed rates increased when IS was increased from 40 nM to 100 nM (FIGS. 9E and 9F). Interestingly, the Twinkle K_{M} obtained from fitting the observed rates to hyperbola remained similar (32±3.24 nM) to the K_{M} measured with 40 nM IS (FIG. 9G). These data indicate that Twinkle concentration required to achieve optimum catalysis is independent of the IS concentration.

Twinkle Requires its DNA Binding Activity, but does not Need its Helicase Activity to Catalyze TMSD

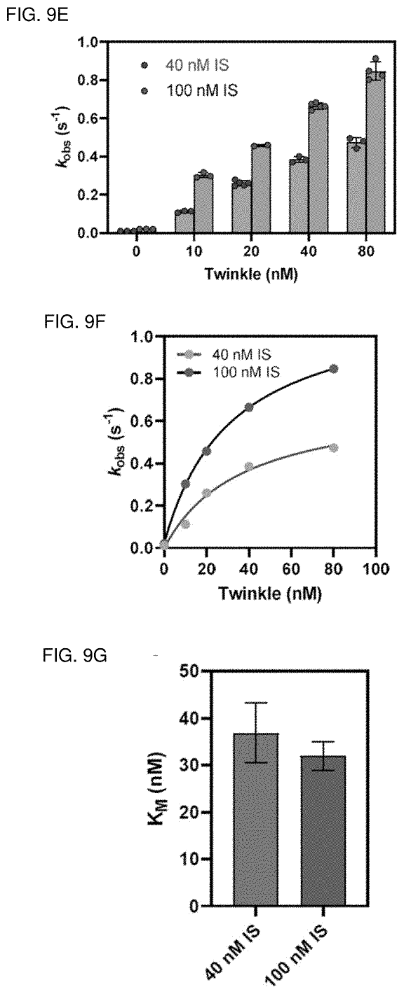

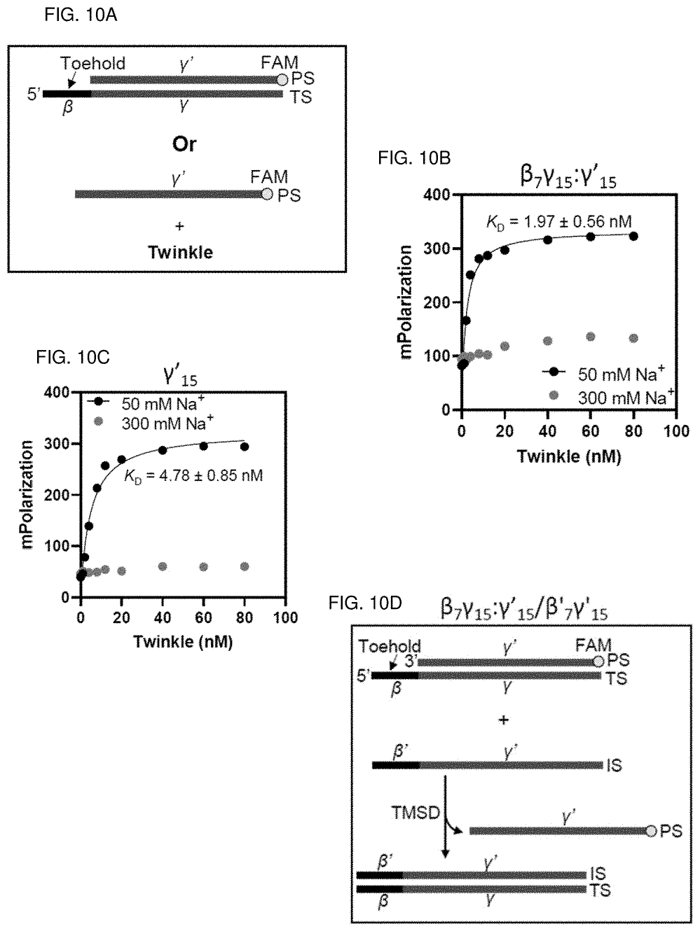

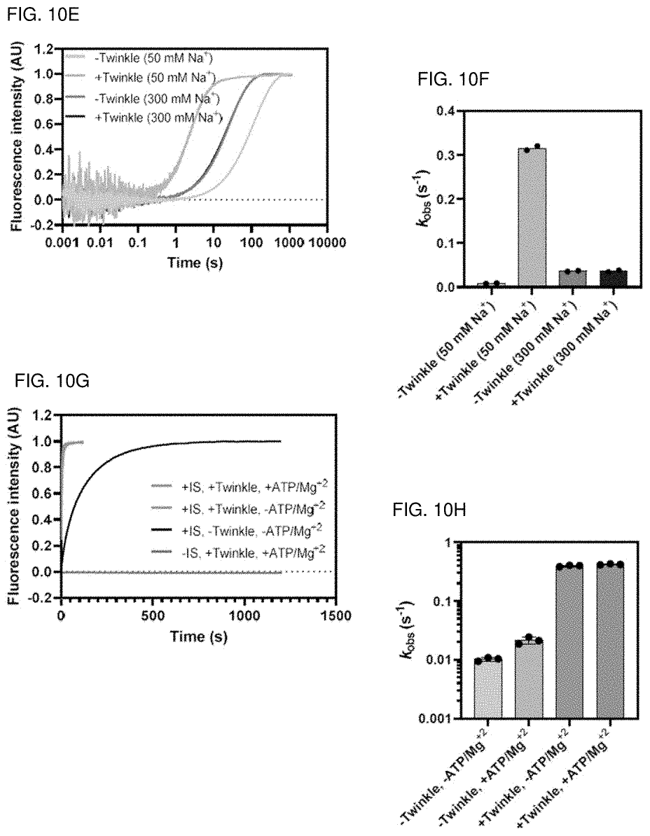

The binding affinity of Twinkle for ssDNA and dsDNA substrates depends on DNA length as well as salt concentration in the buffer^{5,6}. Under high salt conditions, Twinkle exhibits weaker binding to the DNA substrates. We performed fluorescence polarization-based titrations at increasing Twinkle concentrations while keeping the FAM-labeled substrate DNA concentrations constant at 5 nM. Fluorescence polarization was plotted as a function of Twinkle concentration and the data were fitted to hyperbola to obtain the dissociation constant (K_{D}). Either FAM labeled target dsDNA (β₇γ₁₅:γ′₁₅) or FAM labeled PS (β′₇γ′₁₅) (TS:PS and IS, respectively, in the TMSD reactions discussed above) was used (FIG. 10A) and the equilibrium binding reactions were performed under two different sodium acetate concentrations, i.e., 50 mM and 300 mM. Under 50 mM sodium acetate conditions, Twinkle binds tightly to both FAM labeled target dsDNA (FIG. 10B) and PS (FIG. 10C), providing KDs of 2 and 4.8 nM, respectively. Expectedly, there was no apparent DNA binding observed at 300 mM sodium acetate for the Twinkle concentration regime used in the experiments (FIGS. 10B and 10C). These results show that the 300 mM sodium acetate condition is not permissive for Twinkle's DNA binding activity.

We next performed TMSD reactions with β₇γ₁₅:γ′₁₅ target dsDNA and β′₇γ′₁₅ IS under 50 mM and 300 mM sodium acetate conditions (FIGS. 10D and 10E). Interestingly, the observed rate of spontaneous TMSD increased from 0.0083 s⁻¹ at 50 mM salt to 0.036 s⁻¹ at 300 mM salt (FIGS. 10E and 10F). This increase can be attributed to the Na ions shielding the negatively charged phosphate backbone to facilitate DNA annealing. However, the Twinkle-catalyzed TMSD rate decreased from 0.32 s⁻¹ to 0.036 s⁻¹ (at 50 mM and 300 mM sodium acetate, respectively) (FIGS. 10E and 10F, FIG. 1I), indicating that Twinkle lost its ability to catalyze TMSD under these conditions. These results show that Twinkle's DNA binding activity is preferrable for its ability to catalyze TMSD reactions.

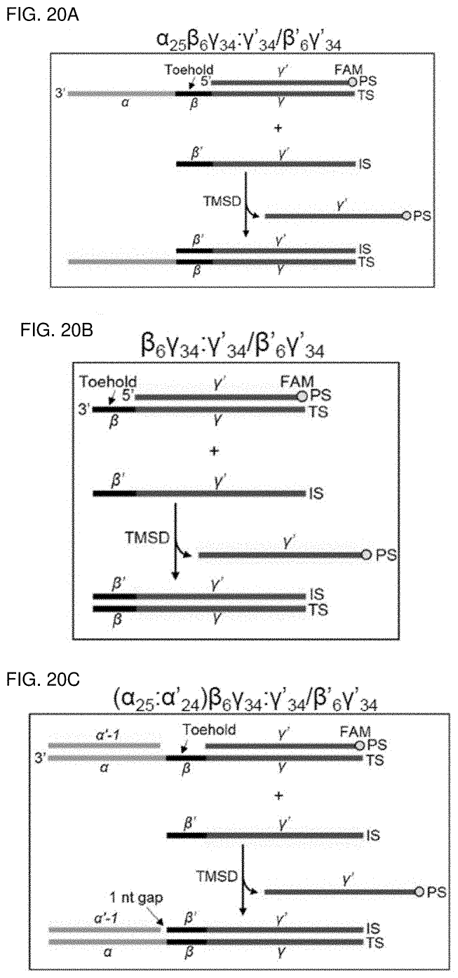

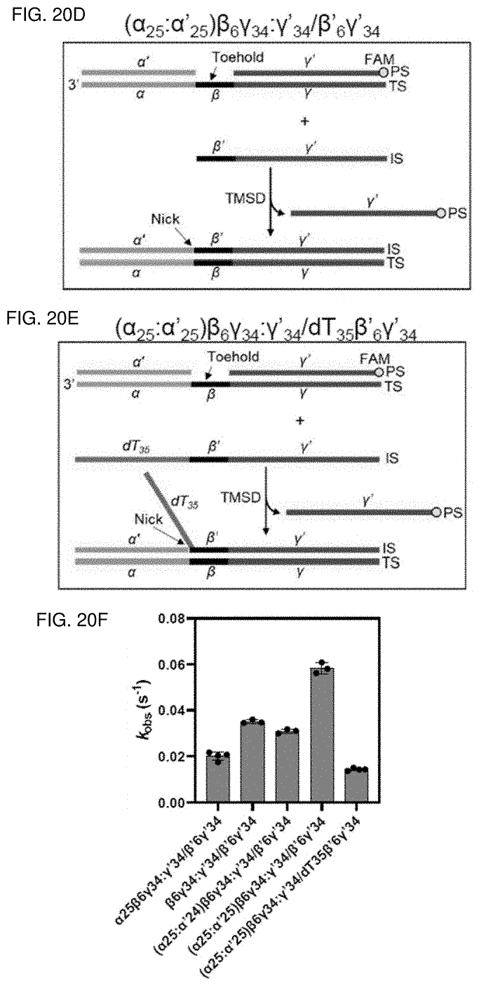

Twinkle uses its ATPase activity (which requires the presence of Magnesium(II) ions) to unwind forked dsDNA substrates in the presence of a ssDNA which traps the unwound ssDNA strand^{6,7}. This ‘strand-exchange’ activity requires NTP hydrolysis by Twinkle and is greatly diminished in absence of a nucleotide 7. To determine whether Twinkle's ATPase activity further stimulates the catalyzed TMSD rates, we measured the spontaneous and Twinkle catalyzed TMSD rates in the presence and absence of ATP and Mg⁺² using the β₇γ₁₅:γ′₁₅ target dsDNA and β′₇γ′₁₅ IS (FIG. 10D). No PS displacement was observed when IS was not added, even in the presence of ATP and Mg⁺². This demonstrates that Twinkle does not unwind the target dsDNA (FIG. 10G). The presence of Mg⁺² and ATP increased the spontaneous TMSD rate from 0.01 s⁻¹ to 0.02 s⁻¹ (FIGS. 10G and 10H). This increase is most likely an outcome of Mg⁺² ions shielding the backbone phosphates to facilitate faster toehold docking. Interestingly, the presence of Mg⁺² and ATP had no effect on Twinkle-catalyzed TMSD rate (FIGS. 10G and 20H). Our results indicate that Twinkle does not use its helicase activity to catalyze the TMSD reaction. These results also show that the conditions favoring the helicase activity of Twinkle do not inhibit Twinkle-catalyzed TMSD, thereby indicating that Twinkle can catalyze TMSD-like reactions in vivo in presence of cellular nucleotides and Mg⁺².

### Twinkle Catalyzes Toehold Hybridization

There are two steps of the TMSD reaction—toehold formation or docking, and branch migration. At low concentrations of target dsDNA and IS, the uncatalyzed TMSD kinetics is bimolecular and limited by the toehold docking step. In contrast, at high concentrations of DNA substrates, the observed rates of spontaneous TMSD also get affected by the length and sequence of the branch migration domain¹⁹. Twinkle has been shown to catalyze annealing of two complementary ssDNA strands. Since Toehold formation is driven by the base-pairing energy of the toehold region, it is essentially an annealing reaction indicating that Twinkle's annealing activity might be involved in accelerating the TMSD reactions. Our data indicates that Twinkle catalyzes DNA annealing by positioning the complementary DNA strands in close proximity, thus facilitating accelerated base-pairing.

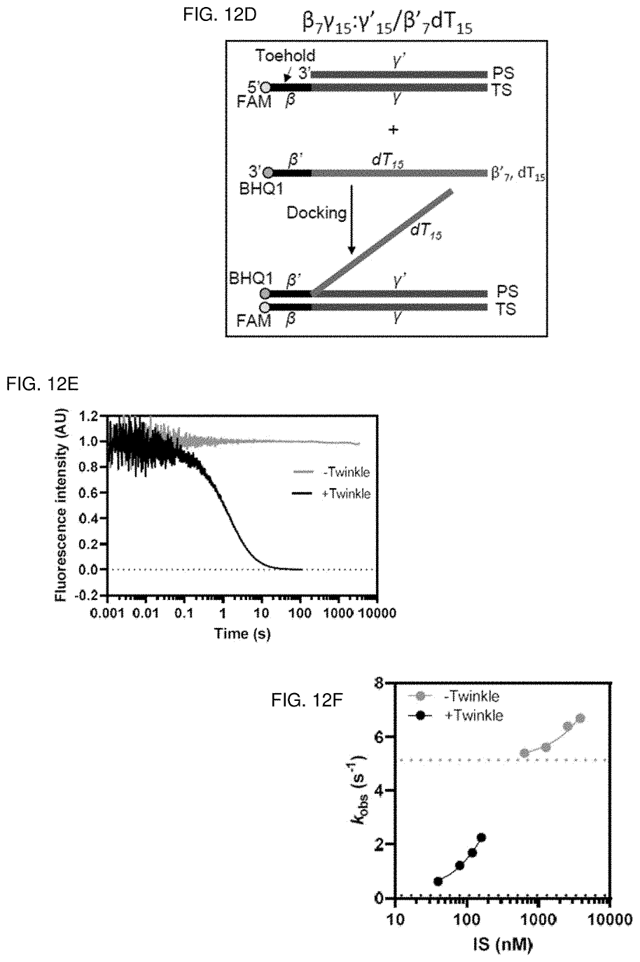

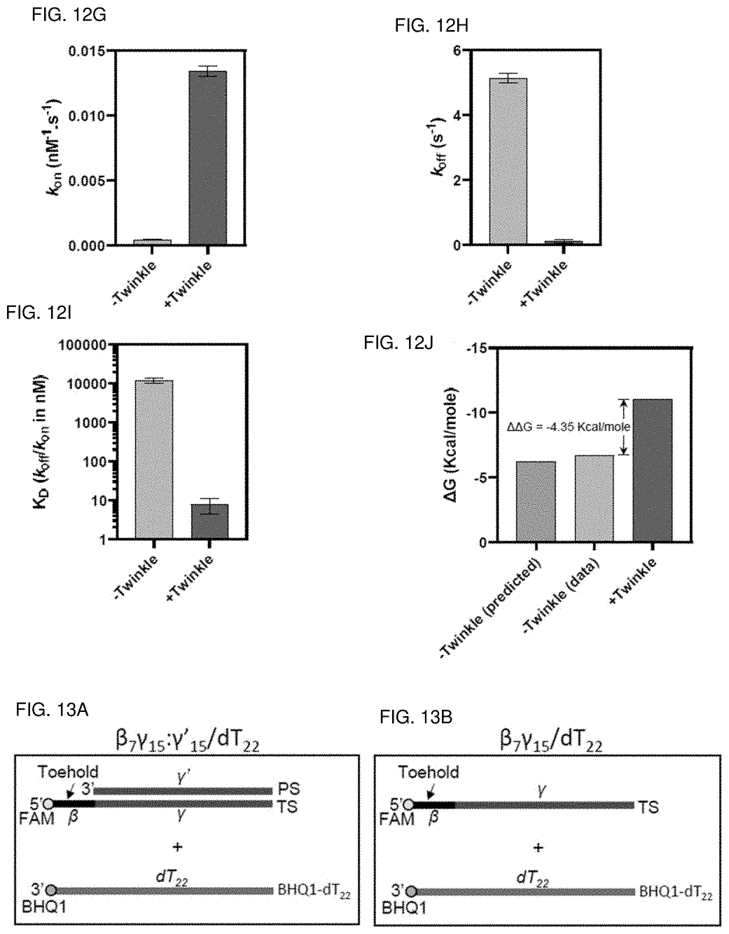

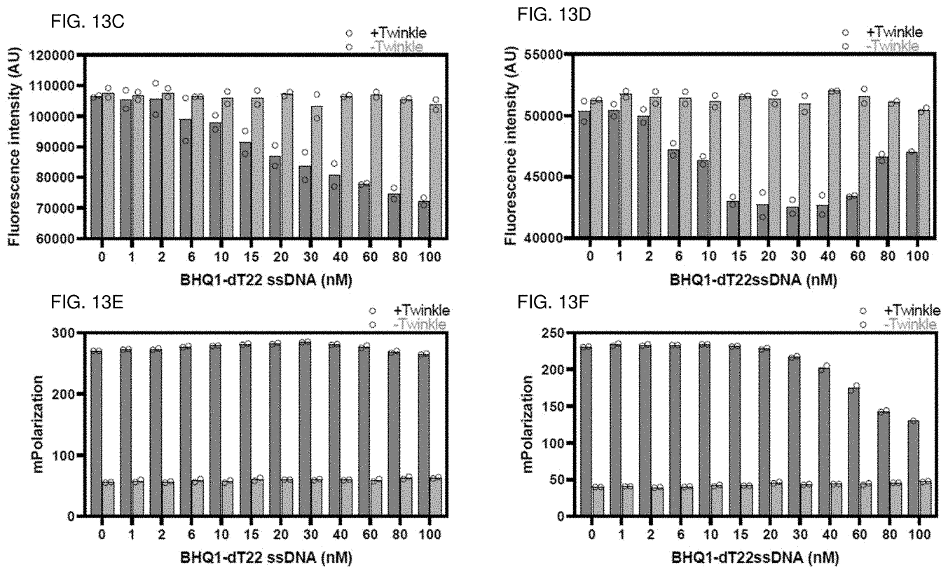

To directly observe if Twinkle has accessory DNA binding sites in addition to the central cavity, and whether it can bring the bound target dsDNA and non-complementary ssDNA, we used the following approach. We labeled the 5′-end of the β₇γᵢₛ TS with FAM and annealed it to an unlabeled γ′₁₅ PS to prepare a dsDNA substrate (β₇γ₁₅:γ′₁₅) with 7 nucleotide overhang, just as in the TMSD target dsDNA used above. We incubated 40 nM of the FAM labeled dsDNA with 40 nM Twinkle hexamer to form a Twinkle-dsDNA complex and titrated a 22 nucleotide dT ssDNA (dT₂₂) labeled with Black Hole Quencher 1 (BHQ1) (FIG. 12A). BHQ1 strongly quenches FAM fluorescence in a distance dependent manner, providing an effective method to assess the proximity of the FAM and BHQ1 labeled DNAs. As the concentration of dT₂₂ ssDNA was increased from 0 to 100 nM, the fluorescence intensity of the Twinkle-dsDNA complex decreased in a hyperbolic manner, providing a K_{D} of 27.73±3.82 nM (FIG. 12B, FIGS. 13A and 13C). The decrease in fluorescence intensity indicates that the BHQ1 labeled dT₂₂ was in close proximity to the FAM labeled dsDNA. The fluorescence intensity of the FAM labeled dsDNA was not affected when similar BHQ1-dT₂₂ ssDNA concentration regime was added in absence of Twinkle, demonstrating that Twinkle acts a scaffold to bring the two DNAs closer (FIG. 12B, FIGS. 13A and 13C).

To ensure that the observed fluorescence decrease happened while the FAM labeled dsDNA was bound to Twinkle, we also measured fluorescence polarization. The dsDNA exhibited high fluorescence polarization when it is bound to Twinkle as compared to the free dsDNA. Within the BHQ1-dT₂₂ concentration used, the polarization did not change much, showing that FAM labeled dsDNA stayed bound with Twinkle even in the presence of the ssDNA (FIG. 12C, FIG. 13E). These results show the formation of dsDNA-Twinkle-ssDNA ternary complex. The reactions without Twinkle did not show appreciable change in polarization (FIG. 12C, FIG. 13E). Similar fluorescence intensity and anisotropy measurements replacing FAM labeled β₇γ₁₅:γ′₁₅ dsDNA with just FAM-labeled β₇γ₁₅ ssDNA showed that Twinkle also brings two ssDNA strands closer, although the BHQ1-dT₂₂ ssDNA competes with the FAM-labeled ssDNA for Twinkle binding (FIGS. 13B, 13D and 13F).

Twinkle is able to bring a toehold containing dsDNA and a ssDNA closer which appears to enhance catalysis of toehold formation. To exclusively measure the kinetics of toehold formation (docking), we designed a 3′ BHQ1 labeled 22-nt β′₇dT₁₅ IS with 7 nucleotides complementary to the toehold in the FAM labeled β₇γ₁₅:γ′₁₅ dsDNA. The rest of the 15 nucleotides in the ssDNA were dT residues. Thus, the β′₇dT₁₅ IS could anneal to the toehold but had complementary domain to perform branch migration (FIG. 12D). The rate of association of the β₇γ₁₅:γ′₁₅ dsDNA and BHQ1-β′₇dT₂₂ ssDNA was measured as the fluorescence decrease in real-time using a stopped-flow device (FIG. 12E). A complex of 10 nM dsDNA and 40 nM Twinkle (final concentrations in the reaction) was loaded in one syringe while increasing concentrations of BHQ1-β′₇dT₂₂ IS were mixed to the reaction through the second syringe. As toehold formation is a bimolecular reaction depending on concentrations of both the DNAs, the observed rate of toehold formation increased linearly with IS concentration (FIG. 12F). While the slopes of the linear fits of the data provided the bimolecular rate of toehold formation, the Y-axis intercept provided the estimates for the ‘off-rates’ (FIGS. 12F-12H). Twinkle increased the ‘on-rate’ (kₒₙ) of toehold formation by 31 times (FIG. 12G) and reduced the ‘off-rate’ (k_{off}) by 49 times (FIG. 12H), thereby tightening the k_{D} (k_{off}/kₒₙ) from 1.1 μM to 7 nM (FIG. 12I). Thus, for this particular set of DNA substrates, Twinkle makes the docking reaction thermodynamically favorable with overall ΔΔG of −4 kcal·mole⁻¹ (FIG. 12J).

### Twinkle Accelerates TMSD by Catalyzing Toehold Formation

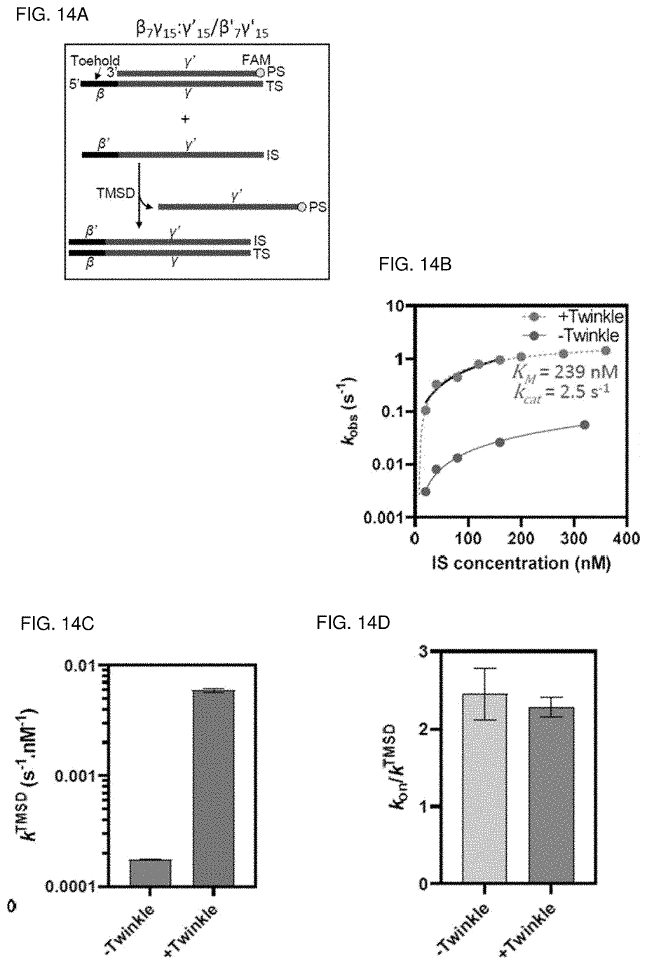

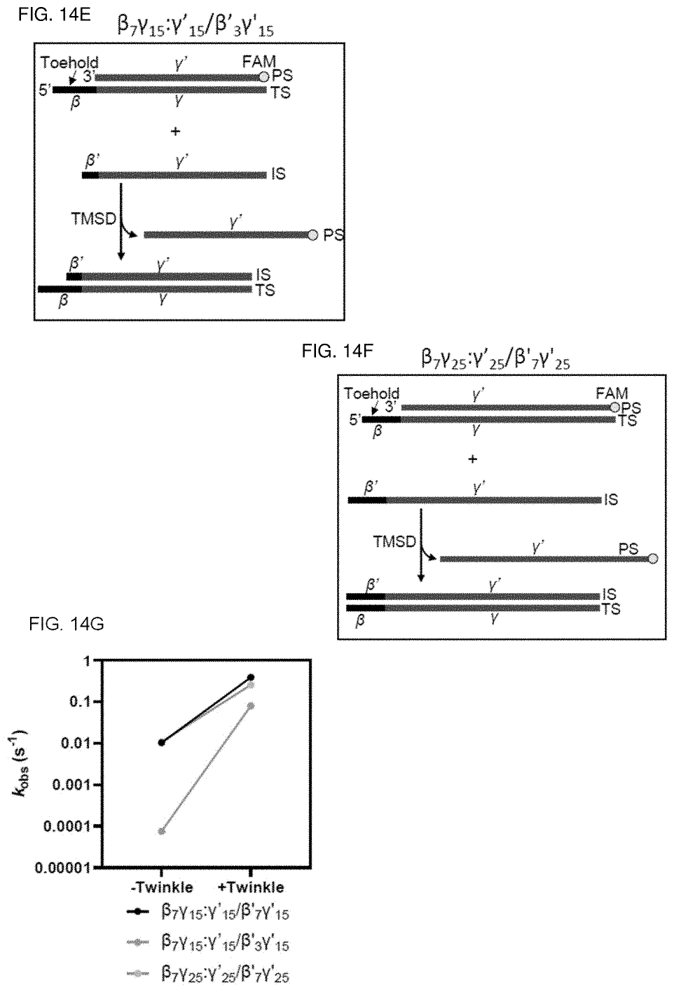

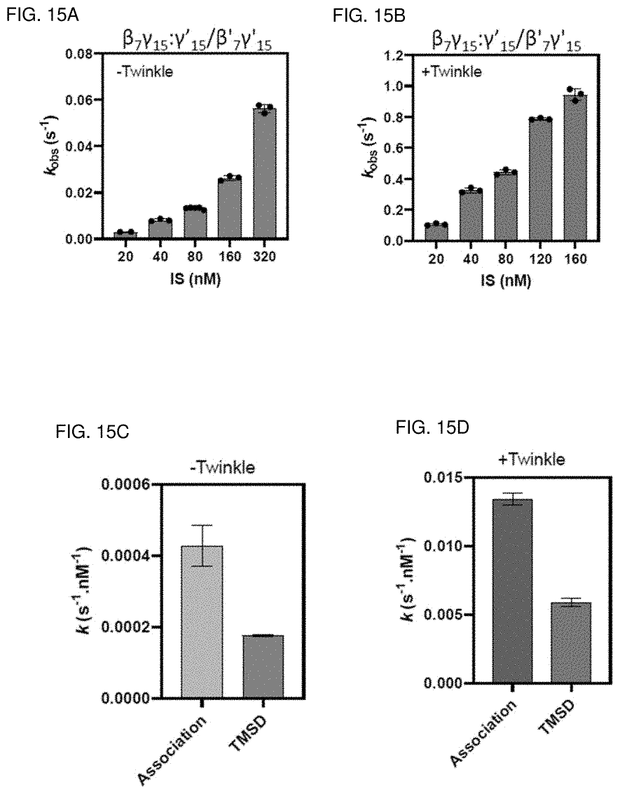

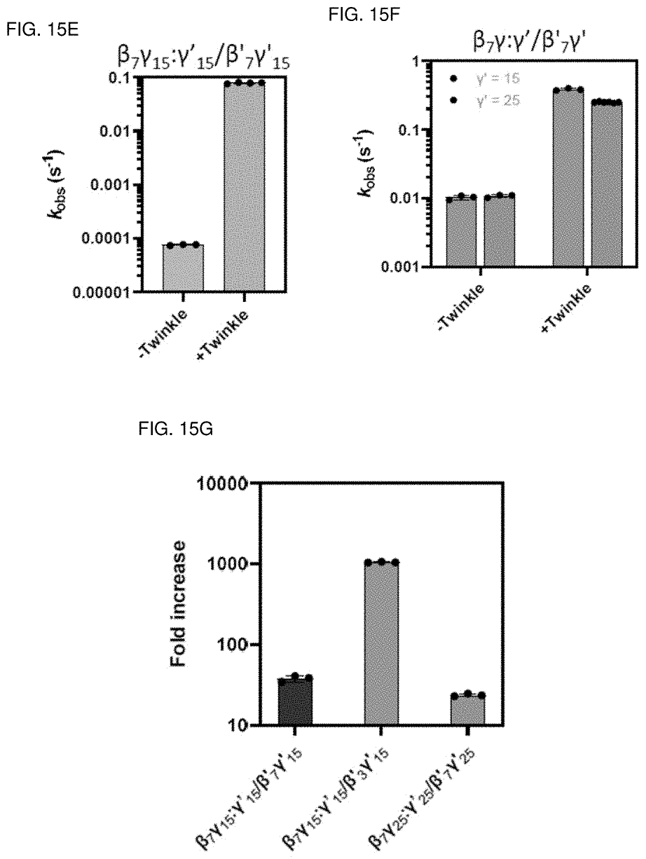

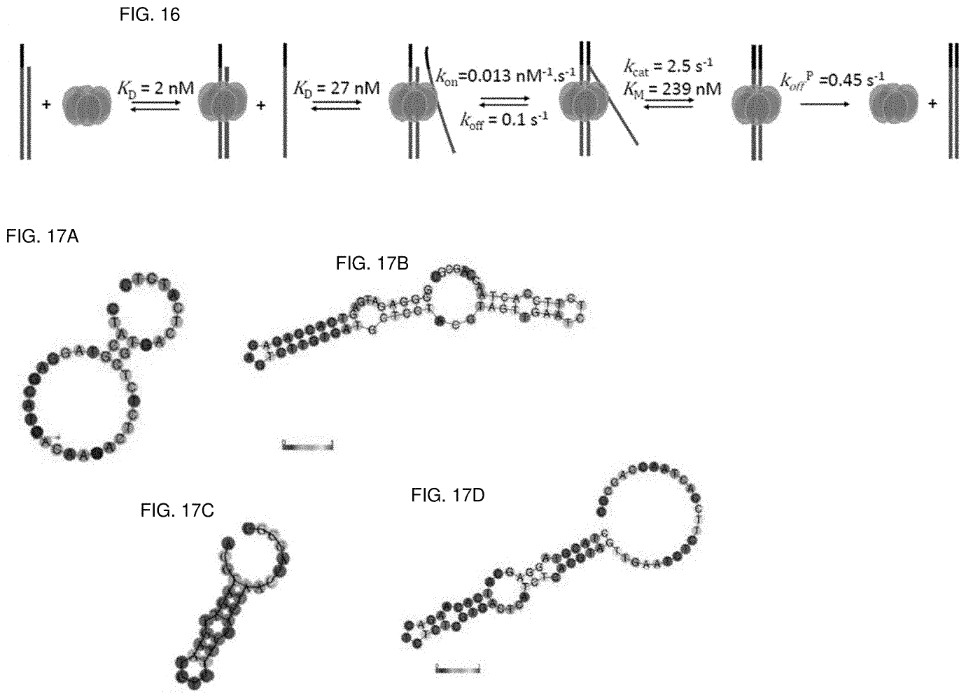

To further understand how Twinkle's annealing activity affects the overall TMSD rates, we determined bimolecular rates of TMSD by varying IS concentration. For this, we used β₇γ₁₅:γ′₁₅ target dsDNA and β′₇γ′₁₅ IS (FIG. 14A). The observed rate for TMSD in absence of Twinkle increased linearly with IS concentration providing a bimolecular rate constant (k^{TMSD}) of 0.00017 nM⁻¹·s⁻¹ for spontaneous TMSD (FIGS. 14B and 14C, FIG. 16A). The observed rates for Twinkle catalyzed TMSD reactions also increased linearly from 10 nM to 180 nM IS providing k^{TMSD} of 0.006 nM⁻¹·s⁻¹ for this range of IS concentration (FIGS. 14B and 14C FIG. 15B). Interestingly, the ratio of bimolecular rates of toehold formation and TMSD (kₒₙ/k^{TMSD}) in presence and absence of Twinkle remained similar (2.45±0.34 and 2.28±0.13, respectively) (FIG. 14D, FIGS. 15C and 15D). Thus, the rate of toehold formation and the rate of the TMSD were similarly accelerated by Twinkle (˜32 times), indicating that the Twinkle catalyzes TMSD reactions by accelerating toehold docking. Conversely, these results also indicate that Twinkle has little impact on accelerating the branch migration, if any.

Increasing the IS concentration beyond 180 nM in Twinkle catalyzed TMSD reactions resulted into hyperbolic increase in observed rates of TMSD, providing a K_{M} of 239±41 nM and maximum rate (k_{cat}) of 2.5 s⁻¹ (FIG. 14B). The large difference between K_{D} of IS binding to the target dsDNA-Twinkle complex (27 nM) and K_{M} (239 nM) indicates that Twinkle catalyzed TMSD follows Briggs-Haldane enzyme kinetics 22. The estimations using kₒₙ, k_{off} and k_{cat} or kₒₙ, K_{D} and k_{cat} provided K_{M} values of 220 nM and 200 nM, respectively, which are within the margin of error from the directly measured K_{M} (239 nM). These results also predicted the rate of branch migration for these TMSD substrates to be ˜2.5 s⁻¹, which is within the range of branch migration rates directly measured in a recent single molecule fluorescence-based study²³.

We conducted additional TMSD reactions to tease out Twinkle's contribution in accelerating toehold docking and branch migration. In the first set of reactions, we changed the toehold length from 7 nucleotides to 3 nucleotides by cutting short the IS by four nucleotides at its 3′-end (β′₃γ′₁₅) while keeping the branch migration domain in the substrate dsDNA same as in the previous experiments (β₇γ₁₅:γ′₁₅) (FIG. 14E). The loss of four nucleotides at the toehold lead to ˜150 fold decrease in spontaneous TMSD rate (k_{obs}=0.000075 s⁻¹). In contrast, the observed rate of Twinkle catalyzed TMSD reduced just by 5 folds from 0.38 s⁻¹ to 0.078 s⁻¹ (FIG. 14G, FIG. 15E). Thus, for a 3-nucleotide toehold substrate, Twinkle accelerated the TMSD rates by more than 1000-fold (versus ˜40 when the toehold was 7-nucleotides long) (FIG. 15G). These results not only demonstrate Twinkle's advantage in catalyzing short toehold reactions, but also verify that it accelerates the TMSD rates by catalyzing the docking step.

In another set of the reactions, we kept the toehold length constant at 7-nt while increasing the length of branch-migration domain by 10 bp (from 15 bp to 25 bp, with target dsDNA β₇γ₂₅:γ′₂₅ and IS β′₇γ′₂₅) (FIG. 14F). The longer displacement domain did not affect the observed rate of spontaneous TMSD (FIG. 14G, FIG. 15F). This was expected as the TMSD reaction rates with lower DNA concentrations are limited by toehold formation followed by fast branch migration. The rates become sensitive to the displacement domain length only when high concentrations of DNAs are used 19. The longer displacement domain reduced the observed rate of Twinkle catalyzed TMSD to 0.23 s⁻¹, around 1.6 times slower than the shorter target dsDNA (β₇γ₁₅:γ′₁₅) (FIG. 14G, FIG. 15G). These results mimic the increased dependency of rates of spontaneous TMSD reactions conducted at high DNA concentrations on displacement domain and indicate that while Twinkle substantially accelerates toehold formation, it does not directly catalyze branch migration¹⁹.

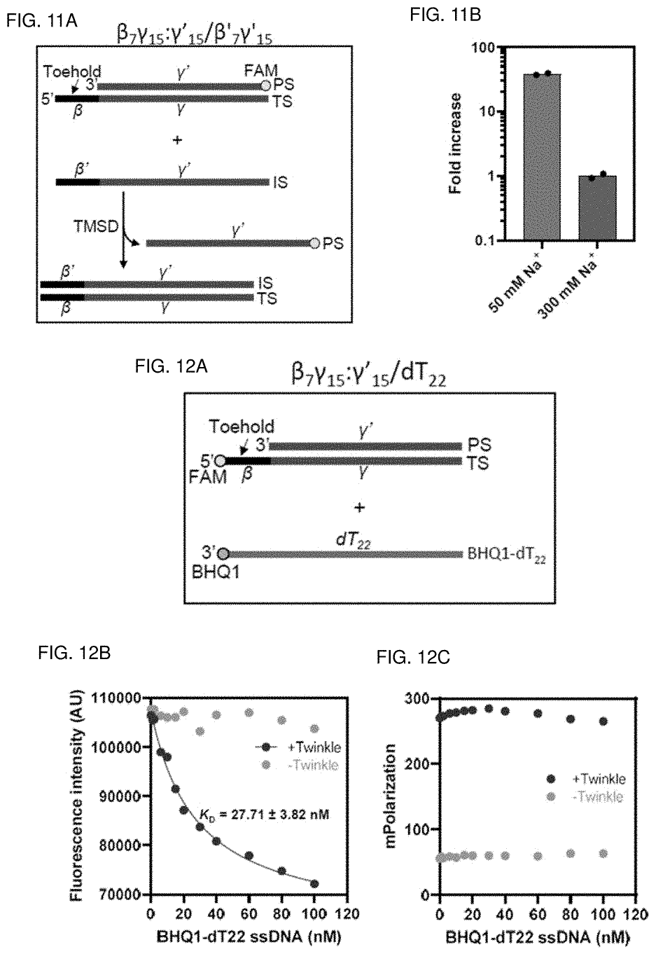

We used fluorescence change in response to Twinkle binding to the FAM labeled TMSD product (β₇γ₁₅:β′₇γ′₁₅) to determine k_{off} for the product dissociation from Twinkle (k_{off}^{P}=0.45 s⁻¹). The relatively fast k_{off}^{P} allows for the possibility for multiple turnovers of the Twinkle catalyzed TMSD. The kinetic framework of the Twinkle catalyzed TMSD derived from the data presented in FIGS. 9-15 has been shown in FIG. 16.

### Twinkle Accelerates TMSD Slowed Down by DNA Secondary Structures.

Prevalent models describing the bimolecular kinetics of TMSD rely on measurements conducted on short TMSD substrates devoid of unnecessary secondary structures^{18,19,24}. Long DNA substrates, their high concentration, and presence of DNA secondary structures limit the efficacy of these models in predicting the kinetic outcomes^{18,19}. Bimolecular DNA hybridization at the toehold may be challenged by intramolecular interactions within the ssDNA strands to form hairpins, loops and stems, and other secondary structures²⁴⁻²⁷. Moreover, the presence of secondary structures in the displacement domain further complicates the energy landscape for branch migration, slowing down the TMSD rates as 4-way branch migration is, in general, slower^{19,24,26}.

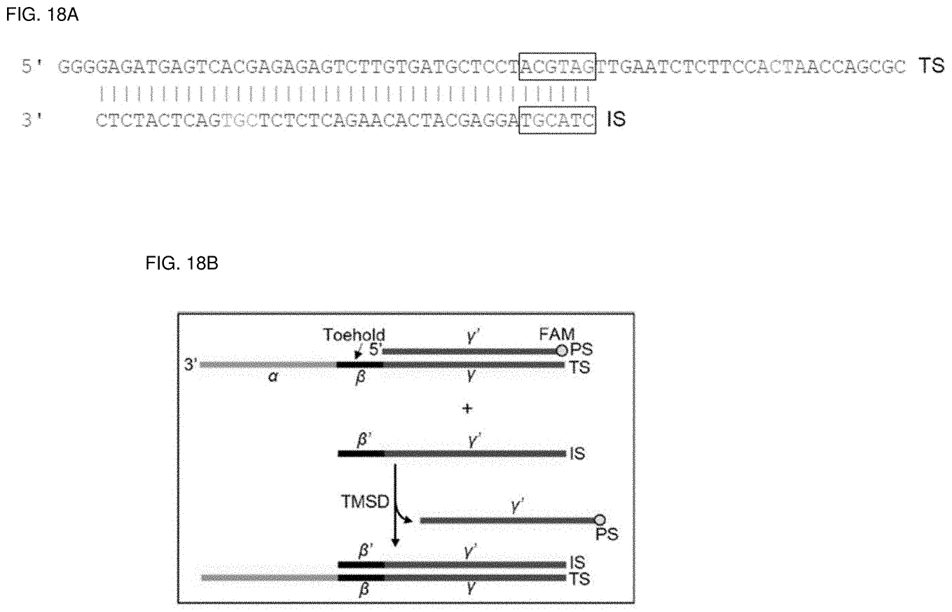

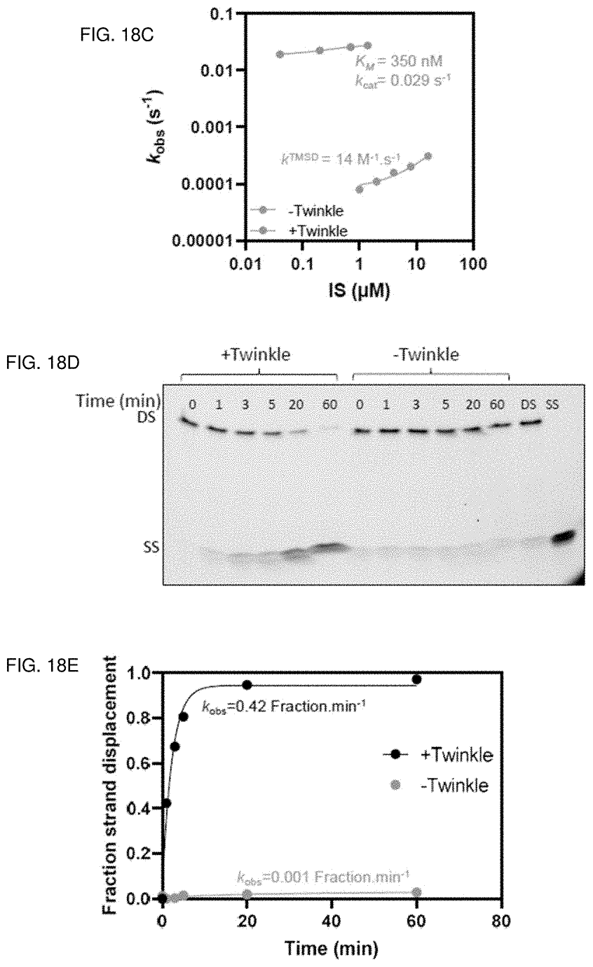

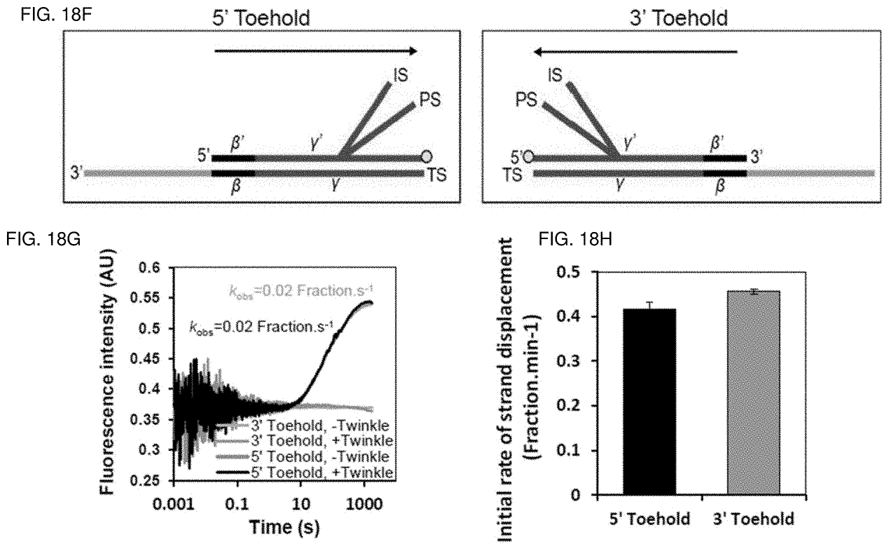

To test effectiveness of Twinkle in catalyzing TMSD on such challenging substrates, we designed a target dsDNA with short, 6-nucleotide toehold domain (β₆) and a longer, 34-bp long branch migration domain (γ₃₄). Further, we extended the 3′-end of the TS by 25-nucleotides (α₂₅) beyond the toehold domain. TS (α₂₅β₆γ₃₄), IS (β′₆γ′₃₄) and 25-nucleotide extension (α₂₅), all formed secondary structures in their single stranded form (FIG. 17). Three of the nucleotides in the toehold domain of IS formed a 3-bp stem, reducing the available nucleotides to participate in toehold formation to three. Additionally, single-stranded α₂₅β₆ portion of the target dsDNA (α₂₅β₆γ₃₄:γ′₃₄) also forms a strong secondary structure that include the three nucleotides in the toehold domain. Assuming that all the secondary structures are stably formed, the effectively available toehold would just be of a single nucleotide (FIG. 18A). But, the secondary structures in the displacement domain would also substantially slow down the TMSD rates.

We used fluorescence-based spectroscopic assay to measure the observed rates of spontaneous TMSD (on α₂₅β₆γ₃₄:γ′₃₄ target dsDNA) as a function of IS (β′₆γ′₃₄) concentration (FIG. 18B). The reactions were very slow, required high concentrations of IS to be observed (in micromolar range) and hours to reach completion (FIG. 18C). The fitting of the data to linear trend provided a slow bimolecular rate of spontaneous TMSD of 14 M⁻¹·s⁻¹. These rates are in agreement with the TMSD rates measured on a substrate with 50-bp long displacement domain and only one available nucleotide for toehold formation in the IS as reported elsewhere²⁴. Addition of Twinkle to these reactions markedly increased the rates, making it possible to conduct these reactions within minutes' timeframe and at much lower IS concentrations (FIG. 18C). Interestingly, observed rates of catalyzed TMSD increased hyperbolically providing a K_{M} of 350 nM and a maximum rate of 0.029 s⁻¹. Theoretically, it would require more than 2 mM of IS to reach the maximum of 0.029 s⁻¹ for an uncatalyzed reaction, making it impractical for the purposes of most of the TMSD applications.

The slow rates of TMSD on this substrate allowed us to accurately measure the rates using a gel-based assay that can resolve the FAM labeled PS in the duplexed and single-stranded forms through different migrations in the gel matrix. The reaction mixture was loaded on a native polyacrylamide gel after different periods of adding the IS. Before loading on the 20% native gel, 0.5% SDS was added to denature Twinkle. As there is no method to quench the TMSD reaction, we loaded the reactions on a gel while running it at a low voltage of 30 V. The fluorescence intensities of the target dsDNA and the displaced PS were quantified to determine the TMSD reaction rate. The spontaneous TMSD reaction produced only 3% of the PS at the end of one hour (FIG. 18D, right side and FIG. 18E). However, in the presence of Twinkle, free PS was observed within a minute and ˜95% after 60 minutes of reaction (FIG. 18D, left side and FIG. 18E). The TMSD rate in the presence of Twinkle was 0.42 strand·min⁻¹ in comparison to the spontaneous TMSD rate of <0.002 strand·min⁻¹ (FIG. 7E).

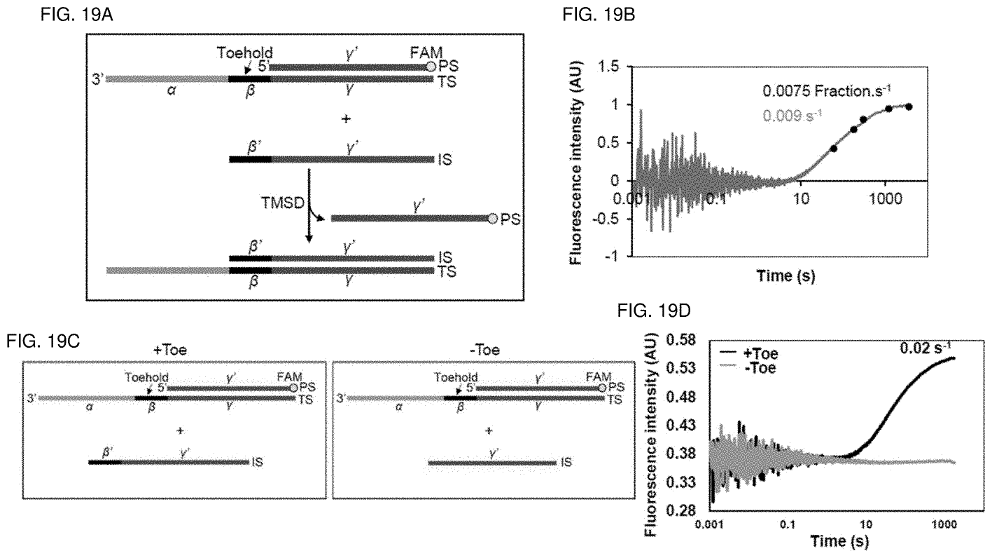

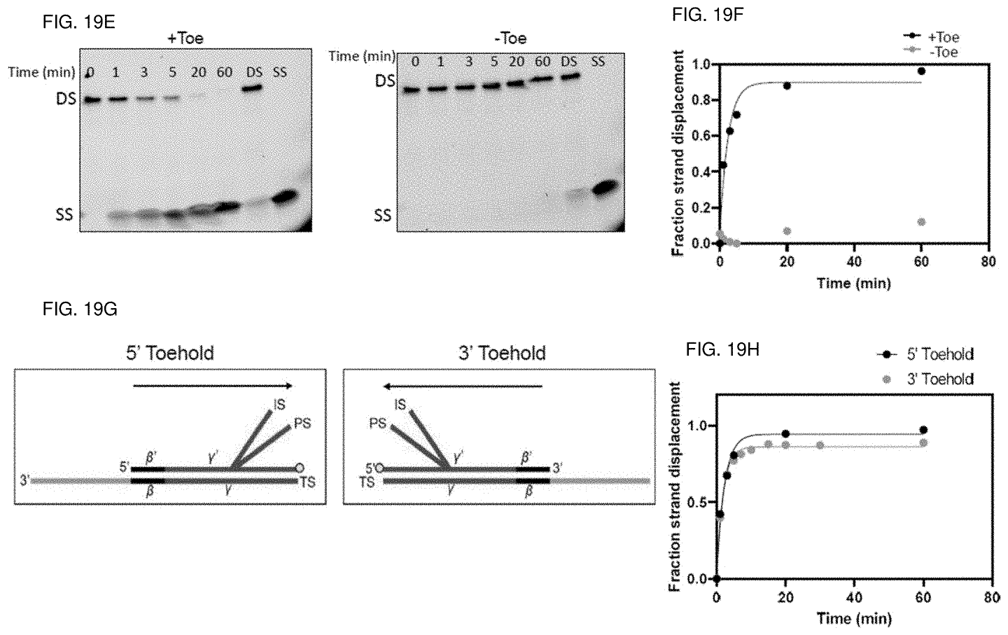

This indicates that Twinkle accelerates the TMSD reaction by >400-fold. These rates matched closely to the TMSD rates measured with our real-time sopped-flow assay performed under similar conditions (FIGS. 19A and 19B). Twinkle requires the toehold region to catalyze these reactions, however limited it might be due to the secondary structures as an IS with no toehold domain (γ′₃₄) did not support strand displacement in the presence or absence of Twinkle (FIGS. 19C-19F).

In the above substrates, the toehold region in the target DNA is at the 3′-end of the 34-bp duplex region. Thus, the IS must displace the PS from the 5′-end to the 3′-end. We designed an alternative target strand of the same nucleotide sequence as the 34-bp substrate used above, but the toehold was placed at the 5′-end of the duplex DNA (FIG. 18F). Twinkle-catalyzed TMSD was tested on this reverse polarity substrate. Twinkle translocates on the DNA in the 5′-3′ direction. If Twinkle's directionality of DNA binding is involved in the TMSD reactions, we would observe a difference in the TMSD rates with opposite polarity substrates (FIG. 18F). Our results show no significant difference in the TMSD rates on the two opposite polarity substrates (˜0.02 s⁻¹ for both the substrate designs) (FIG. 18G). The gel-based assay also confirmed these results. Twinkle-catalyzed TMSD reaction with the reversed polarity substrate was 0.46 strand·min⁻¹, similar to the rates measured for the original substrate design (0.42 strand·min⁻¹) (FIG. 18H and FIGS. 19G and 19H). These results indicate that Twinkle can catalyze TMSD in both directions, from 5′-3′ and 3′-5′.

### Twinkle-Catalyzed TMSD Rates are Controllable by DNA Design Changes

The ability to control kinetics of Twinkle-catalyzed TMSD reactions by changing the substrate design was determined herein. In the 34-bp TMSD substrate described above, there is a 31-nt ssDNA overhang at the 3′-end of the TS (α₂₅β₆) (FIG. 20A). We used this overhang as a handle to fine-tune the kinetics of Twinkle-catalyzed TMSD reactions. We reasoned above that the slow rate of TMSD reaction with this substrate is partly due to the secondary structure formed by the α₂₅β₆ ssDNA region. The impediment in toehold docking step due to secondary structures in α₂₅ overhang can be overcome by the removal of α₂₅. Removing the α₂₅ overhang in the target strand but leaving the 6-nt toehold region (β₆) unchanged (FIG. 20B), increased the observed rate of Twinkle-catalyzed TMSD by ˜1.7-fold (FIG. 20F). Similarly, changing the ssDNA overhang to a duplex DNA by annealing a 24-nt complementary oligo (α′₂₅) without any changes to the toehold region (FIG. 20C) increased the reaction rate by 1.55-fold (FIG. 20F).

The α′₂₄ annealing to the α₂₅ overhang generates a 1-nt gap between α′₂₄ and incoming IS (FIG. 20B). We further modified the target dsDNA by annealing α′₂₅ ssDNA to the α₂₅ overhang, creating a nick between α′₂₅ and IS (FIG. 20D), which increased the rate by over 3-fold (FIG. 20F). This additional acceleration over the 1-nt gap substrate shows the role of base-stacking interactions^{28,29} to stabilize IS on the target dsDNA. Thus, changes in the target strand upstream of the toehold region affects the overall TMSD rates in the presence of Twinkle.

The changes to the IS, affecting the location of the toehold domain, also influenced the kinetics of Twinkle-catalyzed TMSD. Adding a 35-nt dT tail to the 5′-end of the IS changed the location of the toe region from the 5′-end to internal (FIG. 20E). The internal toe reduced the rate of Twinkle-catalyzed TMSD by over 1.4-fold (FIG. 20F). These results indicate that Twinkle-catalyzed TMSD reactions can be fine-tuned by changing the substrate design.

### Twinkle-Catalyzed TMSD is Sensitive to Nucleotide Mismatches

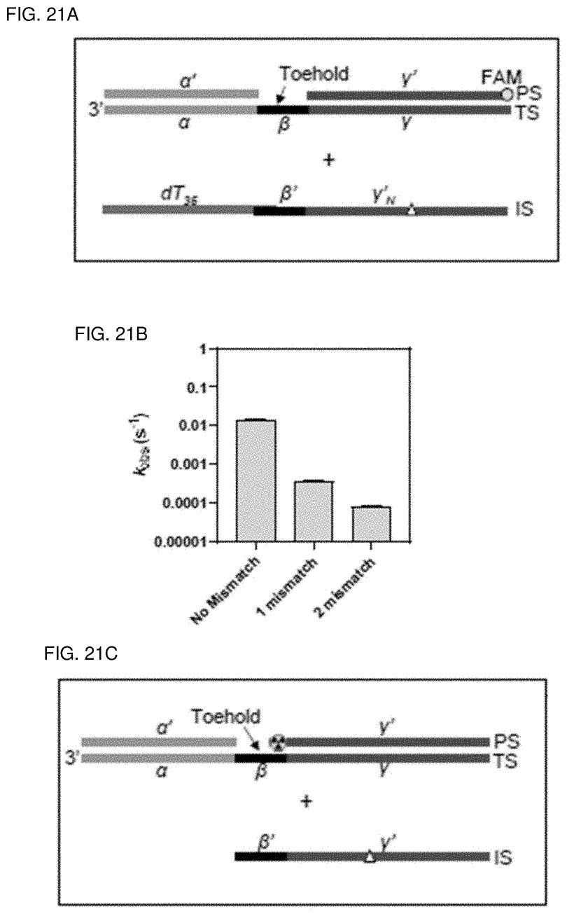

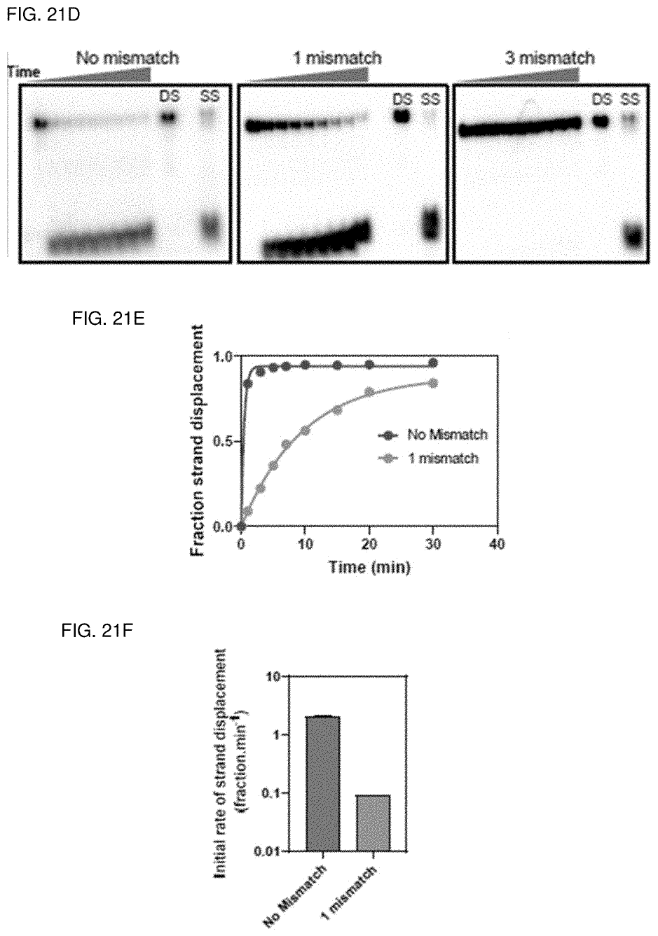

The spontaneous TMSD reaction rates are reported to be susceptible to single-nucleotide mismatches in the branch migration domains of TS and IS^{30,31}. This has been exploited in biotechnological applications for detection of single nucleotide mutations and polymorphisms^{27,32,33}. To determine if Twinkle-catalyzed TMSD is sensitive to mismatches, we designed three dT₃₅β′₆γ′₃₄ IS with 1, 2, or 3 contiguous nucleotide changes that result in mismatches between the IS and the TS. These mismatches were placed approximately in the middle of the branch migration domain (FIG. 21A). Our results show that Twinkle-catalyzed TMSD is highly sensitive to mismatches. The single mismatch reduced the TMSD rate by 38-fold, 2-nt mismatch by over 180-fold, and the 3-nt mismatch showed barely detectable TMSD reaction (FIG. 21B).

We also confirmed these results using the gel-based assay to visualize displacement of radiolabeled PS. The IS used was β′₆γ′₃₄ which created 1-nt or 3-nt mismatch in the branch migration domain (FIG. 21C). The 1-nt mismatch reduced the TMSD rate by over 22-fold and the 3-nt mismatch did not show detectable TMSD reaction (FIGS. 21D-21E).

### Discussion

Nucleic acid strand exchange forms the basis of DNA recombination and repair in cell and CRISPR-Cas based gene editing in vitro. While these exchange reactions are mediated by specialized enzyme complexes, enzyme-free DNA strand displacement is made feasible by providing a short single stranded toehold onto which an invader strand can dock and eventually displace the protector strand through branch migration²⁴. Since the first demonstration of TMSD, the simple reaction has been used to design self-running nanomachines^{10,34}, nanocircuits^{9,11}, biosensors^{14,17,33} genotyping platforms^{27,31-33} etc. and has emerged as an indispensable constituent of DNA based nanotechnology and computation. High specificity of DNA strand hybridization, predictable kinetics and sensitivity to mismatches in the branch migration domain makes TMSD a powerful component in the DNA nanotechnology toolbox¹⁵. However, the major limitation is the slow kinetics of TMSD reaction^{11,16}, which makes its widespread applications restrictive. Most of the mechanistic studies on TMSD rely on short DNA substrates, carefully designed to avoid formation of unnecessary secondary structures^{18,19}. The presence of secondary structures makes the branch migration slow and unpredictable^{18,35}. Furthermore, the long displacement domain often results in unpredictable kinetics¹⁹.

Several enzyme catalyzed strategies have been devised to improve regulation and kinetics of TMSD. DNA nicking enzymes^{36,37} or exonucleases³⁴ can be used to remove a small portion of one of the strands in substrate DNA duplex to create a single stranded toehold and trigger the strand displacement reaction. The main advantage of the strategy is usually to decrease the ‘leakage’ in the DNA based circuits and to introduce a regulatory switch for circuit manipulation. Conversely, strand displacing DNA polymerases can be used to overcome slow speeds of spontaneous TMSD reactions^{16,38}. The strand displacing DNA polymerase accelerates the TMSD by synthesizing the invader strand using target strand as a template. The synthesized DNA eventually displaces the protector strand resulting in accelerated kinetics of the TMSD reactions while reducing the leakage. Expectedly, these reactions are biologically complex and often require both specialized reagents (multiprotein complexes, nucleotides, magnesium, buffer components etc.) and tedious reaction setups.

We demonstrate herein that human mitochondrial helicase Twinkle can catalyze TMSD reactions on a diverse set of DNA substrates. In contrast to the previously reported enzyme catalyzed strand displacement strategies, Twinkle as a single protein accelerates challenging TMSD reactions without involving complexities arising from specialized reagents, other protein partners, or cumbersome reaction designs. Twinkle catalysis also allows rapid TMSD reactions even with non-permissive substrates that otherwise exhibit extremely slow kinetics.

We further elucidate the molecular mechanism of Twinkle's TMSD activity. Apart from the primary binding site where the DNA substrates bind with the K_{D} of 1-5 nM, we directly demonstrate the existence of accessory DNA binding sites where the ssDNA binds with K_{D} of ˜27 nM. Twinkle can utilize its primary and accessary binding modes to simultaneously harbor the target dsDNA and the IS in close proximity. Our data indicates that Twinkle creates a circe effect as proposed by Jencks^{21,39}, juxtaposing the toehold domains of the reacting DNA molecules to accelerate the kinetics of toehold docking. Furthermore, once the toehold is formed, Twinkle does not accelerate or impede branch migration.

Employing the cutting edge biochemical and biophysical approaches, we have determined the quantitative parameters describing the dissociation constants for Twinkle's primary and secondary DNA binding, kₒₙ and k_{off} for toehold formation, K_{M} for the Twinkle catalyzed TMSD and k_{off} for the product dissociation from Twinkle. These measurements outline a complete kinetic framework for the Twinkle catalyzed TMSD and provide a guide to tailor customized TMSD reactions.

Interestingly, akin to the spontaneous TMSD, the kinetics of Twinkle catalyzed TMSD is responsive to the small changes introduced in the substrates including DNA secondary structures, nucleotide mismatches, base stacking interactions and DNA overhangs. We show that by introducing these changes to the DNA substrates, we can tweak and tune the kinetics of the catalyzed TMSD.

### REFERENCES

1 Spelbrink, J. N. et al. Human mitochondrial DNA deletions associated with mutations in the gene encoding Twinkle, a phage T7 gene 4-like protein localized in mitochondria. Nat Genet 28, 223-231, doi:10.1038/90058 (2001).
2 Korhonen, J. A., Pham, X. H., Pellegrini, M. & Falkenberg, M. Reconstitution of a minimal mtDNA replisome in vitro. EMBO J 23, 2423-2429, doi:10.1038/sj.emboj.7600257 (2004).
3 Fernandez-Millan, P. et al. The hexameric structure of the human mitochondrial replicative helicase Twinkle. Nucleic Acids Res 43, 4284-4295, doi:10.1093/nar/gkv189 (2015).
4 Korhonen, J. A., Gaspari, M. & Falkenberg, M. TWINKLE Has 5′->3′ DNA helicase activity and is specifically stimulated by mitochondrial single-stranded DNA-binding protein. J Biol Chem 278, 48627-48632, doi:10.1074/jbc.M306981200 (2003).
5 Johnson, L. C., Singh, A. & Patel, S. S. The N-terminal domain of the human mitochondrial helicase Twinkle has DNA binding activity crucial for supporting processive DNA synthesis by Polymerase γ. 2022.2011.2010.516034, doi:10.1101/2022.11.10.516034% J bioRxiv (2022).
6 Sen, D., Nandakumar, D., Tang, G. Q. & Patel, S. S. Human mitochondrial DNA helicase TWINKLE is both an unwinding and annealing helicase. J Biol Chem 287, 14545-14556, doi:10.1074/jbc.M111.309468 (2012).
7 Sen, D., Patel, G. & Patel, S. S. Homologous DNA strand exchange activity of the human mitochondrial DNA helicase TWINKLE. Nucleic Acids Res 44, 4200-4210, doi:10.1093/nar/gkw098 (2016).
8 Yurke, B., Turberfield, A. J., Mills, A. P., Jr., Simmel, F. C. & Neumann, J. L. A DNA-fueled molecular machine made of DNA. Nature 406, 605-608, doi:10.1038/35020524 (2000).
9 Seelig, G., Soloveichik, D., Zhang, D. Y. & Winfree, E. Enzyme-free nucleic acid logic circuits. Science 314, 1585-1588, doi:10.1126/science.1132493 (2006).
10 Zhang, D. Y., Turberfield, A. J., Yurke, B. & Winfree, E. Engineering entropy-driven reactions and networks catalyzed by DNA. Science 318, 1121-1125, doi:10.1126/science.1148532 (2007).
11 Qian, L. & Winfree, E. Scaling up digital circuit computation with DNA strand displacement cascades. Science 332, 1196-1201, doi:10.1126/science.1200520 (2011).
12 Qian, L., Winfree, E. & Bruck, J. Neural network computation with DNA strand displacement cascades. Nature 475, 368-372, doi:10.1038/nature10262 (2011).
13 Garg, S. et al. Renewable Time-Responsive DNA Circuits. Small, e1801470, doi:10.1002/sm11.201801470 (2018).
14 Sapkota, K., Kaur, A., Megalathan, A., Donkoh-Moore, C. & Dhakal, S. Single-Step FRET-Based Detection of Femtomoles DNA. Sensors (Basel) 19, doi:10.3390/s19163495 (2019).
15 Simmel, F. C., Yurke, B. & Singh, H. R. Principles and Applications of Nucleic Acid Strand Displacement Reactions. Chem Rev 119, 6326-6369, doi:10.1021/acs.chemrev.8b00580 (2019).
16 Song, T. et al. Fast and compact DNA logic circuits based on single-stranded gates using strand-displacing polymerase. Nat Nanotechnol 14, 1075-1081, doi:10.1038/s41565-019-0544-5 (2019).
17 Jung, J. K., Archuleta, C. M., Alam, K. K. & Lucks, J. B. Programming cell-free biosensors with DNA strand displacement circuits. Nat Chem Biol 18, 385-393, doi:10.1038/s41589-021-00962-9 (2022).
18 Srinivas, N. et al. On the biophysics and kinetics of toehold-mediated DNA strand displacement. Nucleic Acids Res 41, 10641-10658, doi:10.1093/nar/gkt801 (2013).
19 Zhang, D. Y. & Winfree, E. Control of DNA strand displacement kinetics using toehold exchange. J Am Chem Soc 131, 17303-17314, doi:10.1021/ja906987s (2009).
20 Wang, B., Thachuk, C., Ellington, A. D., Winfree, E. & Soloveichik, D. Effective design principles for leakless strand displacement systems. Proc Natl Acad Sci USA 115, E12182-E12191, doi:10.1073/pnas.1806859115 (2018).
21 Jencks, W. P. Binding energy, specificity, and enzymic catalysis: the circe effect. Adv Enzymol Relat Areas Mol Biol 43, 219-410, doi:10.1002/9780470122884.ch4 (1975).
22 Briggs, G. E. & Haldane, J. B. A Note on the Kinetics of Enzyme Action. Biochem J 19, 338-339, doi:10.1042/bj0190338 (1925).
23 Broadwater, D. W. B., Jr., Cook, A. W. & Kim, H. D. First passage time study of DNA strand displacement. Biophys J 120, 2400-2412, doi:10.1016/j.bpj.2021.01.043 (2021).
24 Mayer, T., Oesinghaus, L. & Simmel, F. C. Toehold-Mediated Strand Displacement in Random Sequence Pools. 2022.2010.2022.513323, doi:10.1101/2022.10.22.513323% J bioRxiv (2022).
25 SantaLucia, J., Jr. & Hicks, D. The thermodynamics of DNA structural motifs. Annu Rev Biophys Biomol Struct 33, 415-440, doi:10.1146/annurev.biophys.32.110601.141800 (2004).
26 Gao, Y., Wolf, L. K. & Georgiadis, R. M. Secondary structure effects on DNA hybridization kinetics: a solution versus surface comparison. Nucleic Acids Res 34, 3370-3377, doi:10.1093/nar/gk1422 (2006).
27 Khodakov, D. A., Khodakova, A. S., Huang, D. M., Linacre, A. & Ellis, A. V. Protected DNA strand displacement for enhanced single nucleotide discrimination in double-stranded DNA. Sci Rep 5, 8721, doi:10.1038/srep08721 (2015).
28 Vasiliskov, V. A., Prokopenko, D. V. & Mirzabekov, A. D. Parallel multiplex thermodynamic analysis of coaxial base stacking in DNA duplexes by oligodeoxyribonucleotide microchips. Nucleic Acids Res 29, 2303-2313, doi:10.1093/nar/29.11.2303 (2001).
29 Protozanova, E., Yakovchuk, P. & Frank-Kamenetskii, M. D. Stacked-unstacked equilibrium at the nick site of DNA. J Mol Biol 342, 775-785, doi:10.1016/j.jmb.2004.07.075 (2004).
30 Machinek, R. R., Ouldridge, T. E., Haley, N. E., Bath, J. & Turberfield, A. J. Programmable energy landscapes for kinetic control of DNA strand displacement. Nat Commun 5, 5324, doi:10.1038/ncomms6324 (2014).
31 Broadwater, D. W. B., Jr. & Kim, H. D. The Effect of Basepair Mismatch on DNA Strand Displacement. Biophys J 110, 1476-1484, doi:10.1016/j.bpj.2016.02.027 (2016).
32 Khodakov, D. A., Khodakova, A. S., Linacre, A. & Ellis, A. V. Toehold-mediated nonenzymatic DNA strand displacement as a platform for DNA genotyping. J Am Chem Soc 135, 5612-5619, doi:10.1021/ja310991r (2013).
33 Xu, H. et al. Enhanced DNA toehold exchange reaction on a chip surface to discriminate single-base changes. Chem Commun (Camb) 50, 14171-14174, doi:10.1039/c4cc07272c (2014).
34 Qu, X. et al. An Exonuclease III-Powered, On-Particle Stochastic DNA Walker. Angew Chem Int Ed Engl 56, 1855-1858, doi:10.1002/anie.201611777 (2017).
35 Hata, H., Kitajima, T. & Suyama, A. Influence of thermodynamically unfavorable secondary structures on DNA hybridization kinetics. Nucleic Acids Res 46, 782-791, doi:10.1093/nar/gkx1171 (2018).
36 Zhang, C. et al. Nicking-Assisted Reactant Recycle To Implement Entropy-Driven DNA Circuit. J Am Chem Soc 141, 17189-17197, doi:10.1021/jacs.9b07521 (2019).
37 Pan, L. et al. Nicking enzyme-controlled toehold regulation for DNA logic circuits. Nanoscale 9, 18223-18228, doi:10.1039/c7nr06484e (2017).
38 Shah, S. et al. Using Strand Displacing Polymerase To Program Chemical Reaction Networks. J Am Chem Soc 142, 9587-9593, doi:10.1021/jacs.0c02240 (2020).
39 Jencks, W. P. On the attribution and additivity of binding energies. Proc Natl Acad Sci USA 78, 4046-4050, doi:10.1073/pnas.78.7.4046 (1981).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A soluble, stable isolated or purified truncated twinkle enzyme comprising a deletion of an amino terminal domain of the mature form of the twinkle enzyme, wherein said truncated twinkle enzyme exhibits increased solubility and catalyzes toehold mediated strand displacement reactions.

2. The soluble, stable isolated or purified truncated twinkle enzyme of claim 1, comprising the carboxy terminal domain (CTD) of SEQ ID NO: 18.

3. The soluble, stable isolated or purified truncated twinkle enzyme of claim 1, further comprising a sequence tag.

4. The soluble, stable isolated or purified truncated twinkle enzyme of claim 3, wherein said tag is SUMO and comprises SEQ ID NO: 19 or SEQ ID NO: 20.

5. The isolated or purified truncated twinkle enzyme of claim 1, truncated twinkle enzyme comprises one or more non naturally occurring amino acids.

6. The isolated or purified truncated twinkle enzyme of claim 1, further comprising a cleavable linker.

7. The isolated or purified truncated twinkle enzyme of claim 1 affixed to a nanoparticle.

8. A nucleic acid encoding any one of the isolated or purified truncated twinkle enzyme as claimed in claim 1.

9. A host cell comprising the nucleic acid of claim 8.

10. A method for rapid and efficient toe hold mediated strand displacement (TMSD) comprising;
a) contacting a double-stranded DNA complex comprising target strand and an incumbent strand, said target strand comprising overhanging toe-hold sequence which is complementary to a third invading DNA strand, said invading strand being single stranded and complementary to the target strand; with an effective amount of a C terminal variant twinkle enzyme of SEQ ID NO:19 or 20;
b) initiating TMSD under hybridizing conditions such that the complementary invading strand hybridizes with the target strand, creating a DNA complex composed of three strands of DNA, branch migration of the invading strand causing displacement of the incumbent strand.

11. The method of claim 10, for detection of single nucleotide polymorphisms and genetic copy number variations.

12. The method of claim 10 for detection of biomarkers indicative of increased cancer risk.

13. The method of claim 10 for use in genotyping viral or bacterial strains.

14. A kit for practicing the method of claim 10.

15. The kit of claim 14, comprising a purified, stable, soluble CTD operably linked to a SUMO tag of SEQ ID NO: 19 and a buffer suitable for TMSD reactions.

16. The kit of claim 14, further comprising positive and negative control sequence constructs.


SEQUENCE LISTING (33 sequences)

SEQ ID NO: 1 - DNA, 68 bp, synthetic construct
  misc_feature 1..68: note = 5' toehold TS 65
  source 1..68: mol_type = other DNA
ggggagatgagtcacgagagagtcttgtgatgctcctacgtagttgaatctcttccactaaccagcgc

SEQ ID NO: 2 - DNA, 34 bp, synthetic construct
  misc_feature 1..34: note = 5' toehold IncS 34
  misc_feature 34: note = linked to FAM moiety
  source 1..34: mol_type = other DNA
aggagcatcacaagactctctcgtgactcatctc

SEQ ID NO: 3 - DNA, 40 bp, synthetic construct
  misc_feature 1..40: note = 5' toehold IS 40
  source 1..40: mol_type = other DNA
ctacgtaggagcatcacaagactctctcgtgactcatctc

SEQ ID NO: 4 - DNA, 68 bp, synthetic construct
  misc_feature 1..68: note = 3' toehold TS 65
  source 1..68: mol_type = other DNA
cgcgaccaatcaccttctctaagttgatgcatcctcgtagtgttctgagagagcactgagtagagggg

SEQ ID NO: 5 - DNA, 34 bp, synthetic construct
  misc_feature 1..34: note = 3' toehold IncS 34
  misc_feature 1: note = linked to FAM moiety
  source 1..34: mol_type = other DNA
ctctactcagtgctctctcagaacactacgagga

SEQ ID NO: 6 - DNA, 40 bp, synthetic construct
  misc_feature 1..40: note = 3' toehold IS 40
  source 1..40: mol_type = other DNA
ctctactcagtgctctctcagaacactacgaggatgcatc

SEQ ID NO: 7 - DNA, 25 bp, synthetic construct
  misc_feature 1..25: note = TS 25
  source 1..25: mol_type = other DNA
gtggagacgtagggtattgaagggg

SEQ ID NO: 8 - DNA, 15 bp, synthetic construct
  misc_feature 1..15: note = Inc 15
  misc_feature 1: note = linked to FAM moiety
  source 1..15: mol_type = other DNA
cttcaataccctacg

SEQ ID NO: 9 - DNA, 22 bp, synthetic construct
  misc_feature 1..22: note = IS 22
  source 1..22: mol_type = other DNA
cttcaataccctacgtctccac

SEQ ID NO: 10 - DNA, 43 bp, synthetic construct
  misc_feature 1..43: note = TS 40
  source 1..43: mol_type = other DNA
ggggagatgagtcacgagagagtcttgtgatgctcctacgtag

SEQ ID NO: 11 - DNA, 34 bp, synthetic construct; identical to SEQ ID NO: 2
  misc_feature 1..34: note = no toe IS 34
  source 1..34: mol_type = other DNA

SEQ ID NO: 12 - DNA, 25 bp, synthetic construct
  misc_feature 1..25: note = P25
  source 1..25: mol_type = other DNA
gcgctggttagtggaagagattcaa

SEQ ID NO: 13 - DNA, 75 bp, synthetic construct
  misc_feature 1..75: note = IS 40-dT35
  source 1..75: mol_type = other DNA
tttttttttttttttttttttttttttttttttttctacgtaggagcatcacaagactctctcgtgactcatctc

SEQ ID NO: 14 - DNA, 75 bp, synthetic construct
  misc_feature 1..75: note = IS 40-dT35 1 nt mismatch
  source 1..75: mol_type = other DNA
tttttttttttttttttttttttttttttttttttctacgtaggagcatcactagactctctcgtgactcatctc

SEQ ID NO: 15 - DNA, 75 bp, synthetic construct
  misc_feature 1..75: note = IS 40-dT35 2 nt mismatch
  source 1..75: mol_type = other DNA
tttttttttttttttttttttttttttttttttttctacgtaggagcatcacttgactctctcgtgactcatctc

SEQ ID NO: 16 - DNA, 75 bp, synthetic construct
  misc_feature 1..75: note = IS 40-dT35 3 nt mismatch
  source 1..75: mol_type = other DNA
tttttttttttttttttttttttttttttttttttctacgtaggagcatcacttcactctctcgtgactcatctc

SEQ ID NO: 17 - protein, 120 aa, synthetic construct
  REGION 1..120: note = SUMO tag sequence
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGS

SEQ ID NO: 18 - protein, 325 aa, synthetic construct
  REGION 1..325: note = Human Mitochondrial Helicase, Twinkle, C-terminal Domain Construct (TWN-CTD)
LPAWHKSIVSFRQLREEVLGELSNVEQAAGLRWSRFPDLNRILKGHRKGELTVFTGPTGSGKTTFISEYALDLCSQGVNTLWGSFEISNVRLARVMLTQFAEGRLEDQLDKYDHWADRFEDLPLYFMTFHGQQSIRTVIDTMQHAVYVYDICHVIIDNLQFMMGHEQLSTDRIAAQDYIIGVFRKFATDNNCHVTLVIHPRKEDDDKELQTASIFGSAKASQEADNVLILQDRKLVTGPGKRYLQVSKNRFDGDVGVFPLEFNKNSLTFSIPPKNKARLKKIKDDTGPVAKKPSSGKKGATTQNSEICSGQAPTPDQPDTSKRSK

SEQ ID NO: 19 - protein, 445 aa, synthetic construct
  REGION 1..445: note = Human Mitochondrial Helicase, Twinkle, C-terminal Domain Construct (TWN-CTD) with SUMO tag sequence
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSLPAWHKSIVSFRQLREEVLGELSNVEQAAGLRWSRFPDLNRILKGHRKGELTVFTGPTGSGKTTFISEYALDLCSQGVNTLWGSFEISNVRLARVMLTQFAEGRLEDQLDKYDHWADRFEDLPLYFMTFHGQQSIRTVIDTMQHAVYVYDICHVIIDNLQFMMGHEQLSTDRIAAQDYIIGVFRKFATDNNCHVTLVIHPRKEDDDKELQTASIFGSAKASQEADNVLILQDRKLVTGPGKRYLQVSKNRFDGDVGVFPLEFNKNSLTFSIPPKNKARLKKIKDDTGPVAKKPSSGKKGATTQNSEICSGQAPTPDQPDTSKRSK

SEQ ID NO: 20 - DNA, 6628 bp, synthetic construct
  misc_feature 1..6628: note = Human Mitochondrial Helicase, Twinkle, C-terminal Domain Construct (TWN-CTD) inserted in frame with pET 28 Vector SUMO tag Sequence
  source 1..6628: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgggcagcagccatcatcatcatcatcacggcagcggcctggtgccgcgcggcagcgctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggatccctgcctgcctggcacaagtccatcgtatctttccggcagcttcgggaggaggtgctaggagaactgtcaaatgtggagcaagcagctggcctccgctggagccgctttccagacctcaatcgtatcttgaagggacatcgaaagggcgagctgacggtcttcacagggccaacaggcagtggaaagacgacattcatcagtgagtatgccctggatttgtgttcccagggggtgaacacactgtggggtagcttcgagatcagcaatgtgagactagcccgggtcatgctgacacagtttgccgaggggcggctggaagatcaactggacaaatatgatcactgggctgaccgctttgaggacctgcccctctatttcatgactttccatggacagcaaagcatcaggactgtaatagatacaatgcaacatgcagtctacgtctatgacatttgtcatgtgatcatcgacaacctgcagttcatgatgggtcacgagcagctgtccacagacaggatcgcagctcaagactacatcatcggggtctttcggaagtttgcaacagacaataactgccatgtgacactggtcattcacccccggaaagaggatgatgacaaggaactgcagacagcgtccatttttggctcagccaaagcaagccaggaagcagacaatgttctgatcctgcaggacaggaagctggtaaccgggccagggaaacggtatctgcaggtgtccaagaaccgctttgatggagatgtaggtgtcttcccgcttgagttcaacaagaactccctcaccttctccattccaccaaagaacaaggcccggctcaagaagatcaaggatgacactggaccagtggccaaaaagccctcttctggcaaaaagggggctacgacacagaactctgagatttgctcaggccaggcccccactcccgaccagccagacacctccaagcgttcaaagtaaagacaagcttaggtatttattcggcgcaaagtgcgtcgggtgatgctgccaacttagtcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 21 - DNA, 360 bp, synthetic construct
  misc_feature 1..360: note = SUMO tag sequence
  source 1..360: mol_type = other DNA
atgggcagcagccatcatcatcatcatcacggcagcggcctggtgccgcgcggcagcgctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggatcc

SEQ ID NO: 22 - DNA, 981 bp, synthetic construct
  misc_feature 1..981: note = Human Mitochondrial Helicase, Twinkle, C-terminal Domain Construct (TWN-CTD) and termination codon
  source 1..981: mol_type = other DNA
ctgcctgcctggcacaagtccatcgtatctttccggcagcttcgggaggaggtgctaggagaactgtcaaatgtggagcaagcagctggcctccgctggagccgctttccagacctcaatcgtatcttgaagggacatcgaaagggcgagctgacggtcttcacagggccaacaggcagtggaaagacgacattcatcagtgagtatgccctggatttgtgttcccagggggtgaacacactgtggggtagcttcgagatcagcaatgtgagactagcccgggtcatgctgacacagtttgccgaggggcggctggaagatcaactggacaaatatgatcactgggctgaccgctttgaggacctgcccctctatttcatgactttccatggacagcaaagcatcaggactgtaatagatacaatgcaacatgcagtctacgtctatgacatttgtcatgtgatcatcgacaacctgcagttcatgatgggtcacgagcagctgtccacagacaggatcgcagctcaagactacatcatcggggtctttcggaagtttgcaacagacaataactgccatgtgacactggtcattcacccccggaaagaggatgatgacaaggaactgcagacagcgtccatttttggctcagccaaagcaagccaggaagcagacaatgttctgatcctgcaggacaggaagctggtaaccgggccagggaaacggtatctgcaggtgtccaagaaccgctttgatggagatgtaggtgtcttcccgcttgagttcaacaagaactccctcaccttctccattccaccaaagaacaaggcccggctcaagaagatcaaggatgacactggaccagtggccaaaaagccctcttctggcaaaaagggggctacgacacagaactctgagatttgctcaggccaggcccccactcccgaccagccagacacctccaagcgttcaaagtaaaga

SEQ ID NO: 23 - DNA, 40 bp, synthetic construct
  source 1..40: mol_type = other DNA; note = 5' toehold IS 40 1 nt mismatch
ctacgtaggagcatcactagactctctcgtgactcatctc

SEQ ID NO: 24 - DNA, 40 bp, synthetic construct
  source 1..40: mol_type = other DNA; note = 5' toehold IS 40 3nt mismatch
ctacgtaggagcatcacttcactctctcgtgactcatctc

SEQ ID NO: 25 - DNA, 24 bp, synthetic construct
  source 1..24: mol_type = other DNA; note = 5' Toehold alpha'24
gcgctggttagtggaagagattca

SEQ ID NO: 26 - DNA, 22 bp, synthetic construct
  source 1..22: mol_type = other DNA; note = dT22
tttttttttttttttttttttt

SEQ ID NO: 27 - DNA, 14 bp, synthetic construct
  source 1..14: mol_type = other DNA; note = B'7dT15
cttcaatttttttt

SEQ ID NO: 28 - DNA, 18 bp, synthetic construct
  source 1..18: mol_type = other DNA; note = IS Beta3 gamma15
aataccctacgtctccac

SEQ ID NO: 29 - DNA, 35 bp, synthetic construct
  source 1..35: mol_type = other DNA; note = TS Beta7 Gamma25
gtggagacgtagggtattgagatgaacgagagggg

SEQ ID NO: 30 - DNA, 25 bp, synthetic construct
  source 1..25: mol_type = other DNA; note = PS gamma25
ctctcgttcatctcaataccctacg

SEQ ID NO: 31 - DNA, 32 bp, synthetic construct
  source 1..32: mol_type = other DNA; note = IS Beta7 Gamma25
ctctcgttcatctcaataccctacgtctccac

SEQ ID NO: 32 - DNA, 31 bp, synthetic construct
  source 1..31: mol_type = other DNA; note = alpha25 Beta6
cgcgaccaatcaccttctctaagttgatgca

SEQ ID NO: 33 - DNA, 71 bp, synthetic construct
  source 1..71: mol_type = other DNA; note = Beta6 gamma34-alpha25 beta6
cgcgaccaatcaccttctctaagttgatgcactctactcagtgctctctcagaacactacgaggatgcatc